SEQUENCE LISTING (145 sequences)

SEQ ID NO: 1 - DNA, 20 bp, Artificial Sequence
  Description of Artificial Sequence  Synthetic Primer
gagtagaattctaatatcta

SEQ ID NO: 2 - DNA, 20 bp, Artificial Sequence
  Description of Artificial Sequence  Synthetic Primer
gagatattagaattctactc

SEQ ID NO: 3 - DNA, 21 bp, Artificial Sequence
  Description of Artificial Sequence  Synthetic Primer
agtgggattccgcatgctagt

SEQ ID NO: 4 - DNA, 12 bp, Artificial Sequence
  Description of Artificial Sequence  Synthetic Primer
taactagcatgc

SEQ ID NO: 5 - DNA, 20 bp, Artificial Sequence
  Description of Artificial Sequence  Synthetic Primer
ttgcggccgcattnnnnttc

SEQ ID NO: 6 - DNA, 22 bp, Artificial Sequence
  Description of Artificial Sequence  Synthetic Primer
ccgactcgacnnnnnnatgtgg

SEQ ID NO: 7 - DNA, 21 bp, Artificial Sequence
  Description of Artificial Sequence  Synthetic Primer
tggtagctcttgatcannnnn

SEQ ID NO: 8 - DNA, 20 bp, Artificial Sequence
  Description of Artificial Sequence  Synthetic Primer
agagttggtagctcttgatc

SEQ ID NO: 9 - DNA, 28 bp, Artificial Sequence
  Description of Artificial Sequence  Synthetic Primer
gtaatacgactcactatagggcnnnnnn

SEQ ID NO: 10 - DNA, 22 bp, Artificial Sequence
  Description of Artificial Sequence  Synthetic Primer
gtaatacgactcactatagggc

SEQ ID NO: 11 - DNA, 18 bp, Artificial Sequence
  Description of Artificial Sequence  Synthetic Primer
gtaatacgactcactata

SEQ ID NO: 12 - DNA, 14 bp, Artificial Sequence
  Description of Artificial Sequence  Synthetic Primer
nncctatagtgagt

SEQ ID NO: 13 - DNA, 15 bp, Artificial Sequence
  Description of Artificial Sequence  Synthetic Primer
nnncctatagtgagt

SEQ ID NO: 14 - DNA, 11 bp, Artificial Sequence
  Description of Artificial Sequence  Synthetic Primer
gacnnnnngtc

SEQ ID NO: 15 - DNA, 12 bp, Artificial Sequence
  Description of Artificial Sequence  Synthetic Primer
nacnnnngtacn

SEQ ID NO: 16 - DNA, 12 bp, Artificial Sequence
  Description of Artificial Sequence  Synthetic Primer
cgannnnnntgc

SEQ ID NO: 17 - DNA, 11 bp, Artificial Sequence
  Description of Artificial Sequence  Synthetic Primer
gccnnnnnggc

SEQ ID NO: 18 - DNA, 10 bp, Artificial Sequence
  Description of Artificial Sequence  Synthetic Primer
gatnnnnatc

SEQ ID NO: 19 - DNA, 11 bp, Artificial Sequence
  Description of Artificial Sequence  Synthetic Primer
ccnnnnnnngg

SEQ ID NO: 20 - DNA, 11 bp, Artificial Sequence
  Description of Artificial Sequence  Synthetic Primer
gcannnnntgc

SEQ ID NO: 21 - DNA, 12 bp, Artificial Sequence
  Description of Artificial Sequence  Synthetic Primer
ccannnnnntgg

SEQ ID NO: 22 - DNA, 12 bp, Artificial Sequence
  Description of Artificial Sequence  Synthetic Primer
gacnnnnnngtc

SEQ ID NO: 23 - DNA, 11 bp, Artificial Sequence
  Description of Artificial Sequence  Synthetic Primer
cctnnnnnagg

SEQ ID NO: 24 - DNA, 10 bp, Artificial Sequence
  Description of Artificial Sequence  Synthetic Primer
gagtcnnnnn

SEQ ID NO: 25 - DNA, 10 bp, Artificial Sequence
  Description of Artificial Sequence  Synthetic Primer
caynnnnrtg

SEQ ID NO: 26 - DNA, 11 bp, Artificial Sequence
  Description of Artificial Sequence  Synthetic Primer
gcnnnnnnngc

SEQ ID NO: 27 - DNA, 11 bp, Artificial Sequence
  Description of Artificial Sequence  Synthetic Primer
ccannnnntgg

SEQ ID NO: 28 - DNA, 10 bp, Artificial Sequence
  Description of Artificial Sequence  Synthetic Primer
gacnnnngtc

SEQ ID NO: 29 - DNA, 13 bp, Artificial Sequence
  Description of Artificial Sequence  Synthetic Primer
ggccnnnnnggcc

SEQ ID NO: 30 - DNA, 15 bp, Artificial Sequence
  Description of Artificial Sequence  Synthetic Primer
ccannnnnnnnntgg

SEQ ID NO: 31 - DNA, 10 bp, Artificial Sequence
  Description of Artificial Sequence  Synthetic Primer
gaannnnttc

SEQ ID NO: 32 - DNA, 20 bp, Artificial Sequence
  Description of Artificial Sequence  Synthetic Primer
gtaatacgactcactatagg

SEQ ID NO: 33 - DNA, 13 bp, Artificial Sequence
  Description of Artificial Sequence  Synthetic Primer
cctatagtgcagt

SEQ ID NO: 34 - DNA, 21 bp, Artificial Sequence
  Description of Artificial Sequence  Synthetic Primer
gtaatacgactcactataggn

SEQ ID NO: 35 - DNA, 14 bp, Artificial Sequence
  Description of Artificial Sequence  Synthetic Primer
ncctatagtgcagt

SEQ ID NO: 36 - DNA, 46 bp, Artificial Sequence
  Description of Artificial Sequence  Synthetic Primer
cctatagtgagtcgtattacttttttgtaatacgactcactatagg

SEQ ID NO: 37 - DNA, 20 bp, Artificial Sequence
  Description of Artificial Sequence  Synthetic Primer
cctatagtgagtcgtattac

SEQ ID NO: 38 - DNA, 21 bp, Artificial Sequence
  Description of Artificial Sequence  Synthetic Primer
agtaatacgactcactatagg

SEQ ID NO: 39 - DNA, 13 bp, Artificial Sequence
  Description of Artificial Sequence  Synthetic Primer
ncctatagtgagt

SEQ ID NO: 40 - DNA, 23 bp, Artificial Sequence
  Description of Artificial Sequence  Synthetic Primer
agtaatacgactcactataggnn

SEQ ID NO: 41 - DNA, 22 bp, Artificial Sequence
  Description of Artificial Sequence  Synthetic Primer
agtaatacgactcactataggn

SEQ ID NO: 42 - DNA, 16 bp, Artificial Sequence
  Description of Artificial Sequence  Synthetic Primer
nnnncctatagtgagt

SEQ ID NO: 43 - DNA, 17 bp, Artificial Sequence
  Description of Artificial Sequence  Synthetic Primer
nnnnncctatagtgagt

SEQ ID NO: 44 - DNA, 22 bp, Artificial Sequence
  Description of Artificial Sequence  Synthetic Primer
gtaatacgactcactataggnn

SEQ ID NO: 45 - DNA, 23 bp, Artificial Sequence
  Description of Artificial Sequence  Synthetic Primer
gtaatacgactcactataggnnn

SEQ ID NO: 46 - DNA, 24 bp, Artificial Sequence
  Description of Artificial Sequence  Synthetic Primer
gtaatacgactcactataggnnnn

SEQ ID NO: 47 - DNA, 25 bp, Artificial Sequence
  Description of Artificial Sequence  Synthetic Primer
gtaatacgactcactataggnnnnn

SEQ ID NO: 48 - DNA, 22 bp, Artificial Sequence
  Description of Artificial Sequence  Synthetic Primer
cgaggcgggtggatcatgaggt

SEQ ID NO: 49 - DNA, 20 bp, Artificial Sequence
  Description of Artificial Sequence  Synthetic Primer
tctgtcgcccaggccggact

SEQ ID NO: 50 - DNA, 28 bp, Artificial Sequence
  Description of Artificial Sequence  Synthetic Primer
ccccccccccgtaatacgactcactata

SEQ ID NO: 51 - DNA, 33 bp, Artificial Sequence
  Description of Artificial Sequence  Synthetic Primer
cccccccccccccccgtaatacgactcactata

SEQ ID NO: 52 - DNA, 38 bp, Artificial Sequence
  Description of Artificial Sequence  Synthetic Primer
ccccccccccccccccccccgtaatacgactcactata

SEQ ID NO: 53 - DNA, 10 bp, Artificial Sequence
  Description of Artificial Sequence  Synthetic Primer
cccccccccc

SEQ ID NO: 54 - DNA, 15 bp, Artificial Sequence
  Description of Artificial Sequence  Synthetic Primer
ccccccccccccccc

SEQ ID NO: 55 - DNA, 20 bp, Artificial Sequence
  Description of Artificial Sequence  Synthetic Primer
cccccccccccccccccccc

SEQ ID NO: 56 - DNA, 21 bp, Artificial Sequence
  Description of Artificial Sequence  Synthetic Primer
cccagaaaccctgagaccctc

SEQ ID NO: 57 - DNA, 21 bp, Artificial Sequence
  Description of Artificial Sequence  Synthetic Primer
tgtgccacaagttaagatgct

SEQ ID NO: 58 - DNA, 22 bp, Artificial Sequence
  Description of Artificial Sequence  Synthetic Primer
tgctgtatcgtgcctgctcaat

SEQ ID NO: 59 - DNA, 21 bp, Artificial Sequence
  Description of Artificial Sequence  Synthetic Primer
tgccccactccccaacattct

SEQ ID NO: 60 - DNA, 21 bp, Artificial Sequence
  Description of Artificial Sequence  Synthetic Primer
aacagagcctcagggaccagt

SEQ ID NO: 61 - DNA, 21 bp, Artificial Sequence
  Description of Artificial Sequence  Synthetic Primer
gggctttgtctgtggttggta

SEQ ID NO: 62 - DNA, 21 bp, Artificial Sequence
  Description of Artificial Sequence  Synthetic Primer
tgggctggctgaggtcaagat

SEQ ID NO: 63 - DNA, 21 bp, Artificial Sequence
  Description of Artificial Sequence  Synthetic Primer
ttttgctccgctgacatttgg

SEQ ID NO: 64 - DNA, 21 bp, Artificial Sequence
  Description of Artificial Sequence  Synthetic Primer
tgctcctgtcccttccacttc

SEQ ID NO: 65 - DNA, 23 bp, Artificial Sequence
  Description of Artificial Sequence  Synthetic Primer
ccttattcccagcagcagtattc

SEQ ID NO: 66 - DNA, 21 bp, Artificial Sequence
  Description of Artificial Sequence  Synthetic Primer
tgggaagggaaagagggtact

SEQ ID NO: 67 - DNA, 21 bp, Artificial Sequence
  Description of Artificial Sequence  Synthetic Primer
ttgctgtagatgggctttcgt

SEQ ID NO: 68 - DNA, 21 bp, Artificial Sequence
  Description of Artificial Sequence  Synthetic Primer
tctgctgggttgatgatttgg

SEQ ID NO: 69 - DNA, 21 bp, Artificial Sequence
  Description of Artificial Sequence  Synthetic Primer
ggcacaagcaaaagggtgtct

SEQ ID NO: 70 - DNA, 21 bp, Artificial Sequence
  Description of Artificial Sequence  Synthetic Primer
ccagcaatcaggaaagcacaa

SEQ ID NO: 71 - DNA, 22 bp, Artificial Sequence
  Description of Artificial Sequence  Synthetic Primer
cacctgtcttgttggcatcacc

SEQ ID NO: 72 - DNA, 23 bp, Artificial Sequence
  Description of Artificial Sequence  Synthetic Primer
ttgttttgcctcaccagtcattt

SEQ ID NO: 73 - DNA, 21 bp, Artificial Sequence
  Description of Artificial Sequence  Synthetic Primer
tcagcaaacccaaagatgtta

SEQ ID NO: 74 - DNA, 21 bp, Artificial Sequence
  Description of Artificial Sequence  Synthetic Primer
ttagtcctttgggcagcacga

SEQ ID NO: 75 - DNA, 21 bp, Artificial Sequence
  Description of Artificial Sequence  Synthetic Primer
tgtctctgcttctgaaacggg

SEQ ID NO: 76 - DNA, 20 bp, Artificial Sequence
  Description of Artificial Sequence  Synthetic Primer
actgccagggtcattgactt

SEQ ID NO: 77 - DNA, 18 bp, Artificial Sequence
  Description of Artificial Sequence  Synthetic Primer
taagcagcaaggtctggg

SEQ ID NO: 78 - DNA, 23 bp, Artificial Sequence
  Description of Artificial Sequence  Synthetic Primer
gtgattgaacaatttggacccac

SEQ ID NO: 79 - DNA, 23 bp, Artificial Sequence
  Description of Artificial Sequence  Synthetic Primer
atggcaacattccacctagtagc

SEQ ID NO: 80 - DNA, 22 bp, Artificial Sequence
  Description of Artificial Sequence  Synthetic Primer
ctccgtcatgataagatgcagt

SEQ ID NO: 81 - DNA, 22 bp, Artificial Sequence
  Description of Artificial Sequence  Synthetic Primer
actgtttggggtgtgaaaggac

SEQ ID NO: 82 - DNA, 20 bp, Artificial Sequence
  Description of Artificial Sequence  Synthetic Primer
actaacaacgccctttgctc

SEQ ID NO: 83 - DNA, 23 bp, Artificial Sequence
  Description of Artificial Sequence  Synthetic Primer
tcagcactccgtatcttcatttg

SEQ ID NO: 84 - DNA, 23 bp, Artificial Sequence
  Description of Artificial Sequence  Synthetic Primer
taaaccgctaaaacgatagcagc

SEQ ID NO: 85 - DNA, 23 bp, Artificial Sequence
  Description of Artificial Sequence  Synthetic Primer
tcatggtattagggaagtgggag

SEQ ID NO: 86 - DNA, 23 bp, Artificial Sequence
  Description of Artificial Sequence  Synthetic Primer
gggaatgaaaagaaaaggcattc

SEQ ID NO: 87 - DNA, 25 bp, Artificial Sequence
  Description of Artificial Sequence  Synthetic Primer
tctttccctctacaaccctctaacc

SEQ ID NO: 88 - DNA, 23 bp, Artificial Sequence
  Description of Artificial Sequence  Synthetic Primer
cagtacatgggtcttatgagtac

SEQ ID NO: 89 - DNA, 22 bp, Artificial Sequence
  Description of Artificial Sequence  Synthetic Primer
aatcgagaacgcacagagcaga

SEQ ID NO: 90 - DNA, 23 bp, Artificial Sequence
  Description of Artificial Sequence  Synthetic Primer
gtctggggagtaaatgcaacatc

SEQ ID NO: 91 - DNA, 20 bp, Artificial Sequence
  Description of Artificial Sequence  Synthetic Primer
ttcttgatgaccctgcacaa

SEQ ID NO: 92 - DNA, 23 bp, Artificial Sequence
  Description of Artificial Sequence  Synthetic Primer
cagcagaagcactaccaaagaca

SEQ ID NO: 93 - DNA, 23 bp, Artificial Sequence
  Description of Artificial Sequence  Synthetic Primer
ttcacctagatggaatagccacc

SEQ ID NO: 94 - DNA, 23 bp, Artificial Sequence
  Description of Artificial Sequence  Synthetic Primer
gcaagatttttgcttggctctat

SEQ ID NO: 95 - DNA, 22 bp, Artificial Sequence
  Description of Artificial Sequence  Synthetic Primer
gaattttggtttcttgctttgg

SEQ ID NO: 96 - DNA, 23 bp, Artificial Sequence
  Description of Artificial Sequence  Synthetic Primer
gtcagaagactgaaaacgaagcc

SEQ ID NO: 97 - DNA, 23 bp, Artificial Sequence
  Description of Artificial Sequence  Synthetic Primer
catctcttgatcatcccagctct

SEQ ID NO: 98 - DNA, 23 bp, Artificial Sequence
  Description of Artificial Sequence  Synthetic Primer
caccattggttgatagcaaggtt

SEQ ID NO: 99 - DNA, 20 bp, Artificial Sequence
  Description of Artificial Sequence  Synthetic Primer
taaacatagcaccaaggggc

SEQ ID NO: 100 - DNA, 23 bp, Artificial Sequence
  Description of Artificial Sequence  Synthetic Primer
tcatgtgtgggtcactaaggatg

SEQ ID NO: 101 - DNA, 20 bp, Artificial Sequence
  Description of Artificial Sequence  Synthetic Primer
cgtctctcccagctaggatg

SEQ ID NO: 102 - DNA, 24 bp, Artificial Sequence
  Description of Artificial Sequence  Synthetic Primer
ctttttcacagaactggtgtcagg

SEQ ID NO: 103 - DNA, 20 bp, Artificial Sequence
  Description of Artificial Sequence  Synthetic Primer
acccagctttcagtgaagga

SEQ ID NO: 104 - DNA, 20 bp, Artificial Sequence
  Description of Artificial Sequence  Synthetic Primer
aatcaaaaggccaacagtgg

SEQ ID NO: 105 - DNA, 18 bp, Artificial Sequence
  Description of Artificial Sequence  Synthetic Primer
actggctgagggagcatg

SEQ ID NO: 106 - DNA, 21 bp, Artificial Sequence
  Description of Artificial Sequence  Synthetic Primer
taaatgtaacccccttgagcc

SEQ ID NO: 107 - DNA, 20 bp, Artificial Sequence
  Description of Artificial Sequence  Synthetic Primer
tattgaccacatgaccccct

SEQ ID NO: 108 - DNA, 21 bp, Artificial Sequence
  Description of Artificial Sequence  Synthetic Primer
ttgggtgatgtcttcacatgg

SEQ ID NO: 109 - DNA, 22 bp, Artificial Sequence
  Description of Artificial Sequence  Synthetic Primer
gctcaataaaaatagtacgccc

SEQ ID NO: 110 - DNA, 20 bp, Artificial Sequence
  Description of Artificial Sequence  Synthetic Primer
ttctcccagctttgagacgt

SEQ ID NO: 111 - DNA, 21 bp, Artificial Sequence
  Description of Artificial Sequence  Synthetic Primer
tttgttacttgctaccctgag

SEQ ID NO: 112 - DNA, 23 bp, Artificial Sequence
  Description of Artificial Sequence  Synthetic Primer
gaagatgaagtgaactcctatcc

SEQ ID NO: 113 - DNA, 22 bp, Artificial Sequence
  Description of Artificial Sequence  Synthetic Primer
gaagccttgataacgagagtgg

SEQ ID NO: 114 - DNA, 19 bp, Artificial Sequence
  Description of Artificial Sequence  Synthetic Primer
atgtttctctggccccaag

SEQ ID NO: 115 - DNA, 17 bp, Artificial Sequence
  Description of Artificial Sequence  Synthetic Primer
tggctgcccttcaatac

SEQ ID NO: 116 - DNA, 20 bp, Artificial Sequence
  Description of Artificial Sequence  Synthetic Primer
ttgggaaatgtcagtgacca

SEQ ID NO: 117 - DNA, 24 bp, Artificial Sequence
  Description of Artificial Sequence  Synthetic Primer
tgtggttaggatagcacaagcatt

SEQ ID NO: 118 - DNA, 22 bp, Artificial Sequence
  Description of Artificial Sequence  Synthetic Primer
tgcaatttgaaggtacgagtag

SEQ ID NO: 119 - DNA, 25 bp, Artificial Sequence
  Description of Artificial Sequence  Synthetic Primer
tgttaacaatttgcataacaaaagc

SEQ ID NO: 120 - DNA, 24 bp, Artificial Sequence
  Description of Artificial Sequence  Synthetic Primer
gcattttctgtcccacaagatatg

SEQ ID NO: 121 - DNA, 20 bp, Artificial Sequence
  Description of Artificial Sequence  Synthetic Primer
attgctgtcacagcaccttg

SEQ ID NO: 122 - DNA, 21 bp, Artificial Sequence
  Description of Artificial Sequence  Synthetic Primer
gcatatccatatctcccgaat

SEQ ID NO: 123 - DNA, 21 bp, Artificial Sequence
  Description of Artificial Sequence  Synthetic Primer
cagagcactccagaccatacg

SEQ ID NO: 124 - DNA, 21 bp, Artificial Sequence
  Description of Artificial Sequence  Synthetic Primer
cttcgttatgacccctgctcc

SEQ ID NO: 125 - DNA, 22 bp, Artificial Sequence
  Description of Artificial Sequence  Synthetic Primer
tcccaagatgaatggtaagacg

SEQ ID NO: 126 - DNA, 21 bp, Artificial Sequence
  Description of Artificial Sequence  Synthetic Primer
tccaatctcatcggtttactg

SEQ ID NO: 127 - DNA, 21 bp, Artificial Sequence
  Description of Artificial Sequence  Synthetic Primer
tccagagcccagtaaacaaca

SEQ ID NO: 128 - DNA, 21 bp, Artificial Sequence
  Description of Artificial Sequence  Synthetic Primer
ttacttcagcccacatgcttc

SEQ ID NO: 129 - DNA, 22 bp, Artificial Sequence
  Description of Artificial Sequence  Synthetic Primer
ttccgacatagcgactttgtag

SEQ ID NO: 130 - DNA, 22 bp, Artificial Sequence
  Description of Artificial Sequence  Synthetic Primer
aaggatcagagataccccacgg

SEQ ID NO: 131 - DNA, 23 bp, Artificial Sequence
  Description of Artificial Sequence  Synthetic Primer
tccaagaaccaactaagtccaga

SEQ ID NO: 132 - DNA, 23 bp, Artificial Sequence
  Description of Artificial Sequence  Synthetic Primer
ctaagggcaaacatagggatcaa

SEQ ID NO: 133 - DNA, 22 bp, Artificial Sequence
  Description of Artificial Sequence  Synthetic Primer
caacctttgaagccactttgac

SEQ ID NO: 134 - DNA, 22 bp, Artificial Sequence
  Description of Artificial Sequence  Synthetic Primer
gcctccgtcattggtattttct

SEQ ID NO: 135 - DNA, 21 bp, Artificial Sequence
  Description of Artificial Sequence  Synthetic Primer
tggcaacacggtgctgacctg

SEQ ID NO: 136 - DNA, 22 bp, Artificial Sequence
  Description of Artificial Sequence  Synthetic Primer
atcatgggtttggcagtaaagc

SEQ ID NO: 137 - DNA, 21 bp, Artificial Sequence
  Description of Artificial Sequence  Synthetic Primer
agaaccagcaaacccagtccc

SEQ ID NO: 138 - DNA, 21 bp, Artificial Sequence
  Description of Artificial Sequence  Synthetic Primer
gaaagggtggatggattgaaa

SEQ ID NO: 139 - DNA, 21 bp, Artificial Sequence
  Description of Artificial Sequence  Synthetic Primer
tcagatttcctggctccgctt

SEQ ID NO: 140 - DNA, 21 bp, Artificial Sequence
  Description of Artificial Sequence  Synthetic Primer
ccttctgcttccctgtgacct

SEQ ID NO: 141 - DNA, 21 bp, Artificial Sequence
  Description of Artificial Sequence  Synthetic Primer
tgaaccccacgaggtgacagt

SEQ ID NO: 142 - DNA, 22 bp, Artificial Sequence
  Description of Artificial Sequence  Synthetic Primer
gacattaccagcccctcaccta

SEQ ID NO: 143 - DNA, 22 bp, Artificial Sequence
  Description of Artificial Sequence  Synthetic Primer
tccttgacagttccattcacca

SEQ ID NO: 144 - DNA, 21 bp, Artificial Sequence
  Description of Artificial Sequence  Synthetic Primer
tttgcaggtagctctaggtca

SEQ ID NO: 145 - DNA, 22 bp, Artificial Sequence
  Description of Artificial Sequence  Synthetic Primer
gcggacagagagtaacctcgga

(19) United States
(12) Patent Application Publication
(10) Pub. No.: US 2004/0209299 A1
(43) Pub. Date: Oct. 21, 2004

(54) In vitro DNA immortalization and whole genome amplification using libraries generated from randomly fragmented DNA

(75) Inventors: Jonathon H. Pinter, Ypsilanti, MI (US); Takao Kurihara, Ann Arbor, MI (US); Irina Sleptsova, Ann Arbor, MI (US); Eric Bruening, Chelsea, MI (US); William Ziehler, Lansing, MI (US); Vladimir L. Makarov, Ann Arbor, MI (US)

Correspondence Address: FULBRIGHT & JAWORSKI, LLP, 1301 MCKINNEY, SUITE 5100, HOUSTON, TX, 77010-3095, US

(73) Assignee: RUBICON GENOMICS, INC.

(21) Appl. No.: 10/797,333

(22) Filed: Mar. 8, 2004

Related U.S. Application Data
(60) Provisional application No. 60/453,071, filed on Mar. 7, 2003.

(51) Int. Cl. C12Q 1/68; C07H 21/04; C12P 19/34

(52) U.S. Cl. 435/6; 435/91.2; 536/25.4

(57) ABSTRACT

The present invention regards a variety of methods and compositions for whole genome amplification. In a particular aspect of the present invention, there is a method of amplifying a genome in a non-biased manner utilizing adaptor-attached randomly generated fragments following modification of the DNA ends prior to the adaptor attachment. In an additional aspect of the present invention, there are methods and compositions for whole genome amplification regarding a one-step endonuclease cleavage and linker ligation reaction.

This application claims priority to the U.S. Provisional Patent Application 60/453,071, filed Mar. 7, 2003, incorporated by reference herein in its entirety.

### FIELD OF THE INVENTION

The present invention is directed to the fields of genomics, molecular biology, genotyping, and molecule diagnostics. In some embodiments, the present invention relates to methods for the amplification of DNA yielding a product that is a non-biased representation of the original genomic sequence, preferably with methods for converting DNA into a library of randomly overlapping, end-linkered fragments. In a particular embodiment, there is a single-reaction method that is suitable for high-throughput library generation.

### BACKGROUND OF THE INVENTION

Genome wide genotyping studies require a large amount of high-quality starting material. Furthermore, the development of clinical diagnostic markers also necessitates a significant quantity of DNA in order to both develop and detect biomarkers of interest, particularly in complex analysis where multiple markers are required to identify specific disease subtypes. However, many clinical and experimental DNA sources are quite limiting and do not provide sufficient material to carry out the necessary studies. Additionally, there exist a large number of stored clinical samples where the history and etiology of the patient is extensively documented. Retrospective studies of this vast source of material and information with modern genotyping technologies may provide a more rapid and cost-effective means of investigating pathology, treatment response, and outcome results than can be obtained by beginning new studies that may require years or decades to complete. The limited quantity and quality of DNA that can be obtained from these samples often precludes their usefulness in large scale genotyping studies. Thus, a method for whole genome amplification (WGA) that can faithfully reproduce the starting DNA in large quantities is needed.

Several methods of WGA have been developed with varying levels of success. These methods can be classified in four ways: ligation mediated PCR™, random primed PCR™, strand displacement mediated PCR™, and cell immortalization. Each of these mechanisms has inherent advantages and disadvantages. The present invention is based on ligation mediated PCR™ and an extensive discussion of this field is presented below. Discussions of random primed PCR™, strand displacement mediated amplification, and cell immortalization methods are also included for comparative purposes.

Ligation Mediated PCR™

The basic premise behind ligation mediated PCR™ is the attachment of specific adaptors to fragments of DNA that are of a suitable size for use in PCR™. These methods were designed to avoid the problems found with using the simpler PCR™ approach described in a later section. The major difficulties in these techniques revolve around three areas: The generation of DNA fragments of the appropriate size representing every region of the genome, the attachment of the adaptors in a sequence-independent manner to both ends of a majority of the DNA fragments, and effective amplification of all fragments without bias. The following techniques have met with varied success in meeting all three requirements.

Representational Difference Analysis (RDA)

The process of Representational Difference Analysis was designed to allow the cloning of differences between two complex genomes (Lisitsyn et al., 1993; Lucito et al., 1998). In this technique, genomic DNA populations were cleaved with rare (6 base pair recognition site, Lisitsyn et al., 1993) or frequent (4 base pair recognition site, Lucito et al., 1998) restriction endonucleases. Adaptors containing overhanging bases complementary to the ends produced by the restriction enzymes were ligated to the digested DNA. In order to avoid self-ligation of adaptors, the adaptor sequences did not contain 5′ phosphate groups. Thus, ligation only occurred between the 3′ end of the adaptor and the 5′ phosphate of the digested DNA. The 3′ ends of the resulting products were subsequently extended to complete the adaptor sequence. PCR amplification of the fragments was carried out to amplify the resulting fragments. The resulting amplified products contained representative levels of DNA fragments that had been cleaved by the restriction endonucleases to yield products of a suitable size for PCR amplification (less than 3 kb, on average). The drawback of this method is that genomic regions lacking in restriction endonuclease recognition sites at frequent intervals (less than 3 kb apart) will not be amplified during PCR. The purpose of this method was not to amplify all sites within the genome, but to amplify many sites for use in subtractive hybridizations for the purpose of determining genomic differences between two samples.

Whole Genome PCR™

Whole genome PCR™ involves converting total genomic DNA to a form that can be amplified by PCR™ (Kinzler and Vogelstein, 1989). In this technique, total genomic DNA is fragmented, via either shearing or restriction with MboI to an average size of 200-300 base pairs. The ends of the DNA are made blunt by incubation with the Klenow fragment of DNA polymerase. The DNA fragments are ligated to catch linkers consisting of a 20 base pair DNA fragment synthesized in vitro. The catch linkers consist of two phosphorylated oligomers: 5′-GAGTAGAATTCTAATATCTA-3′ (SEQ ID NO:1) and 5′-GAGATATTAGAATTCTACTC-3′ (SEQ ID NO:2). To fragment the catch linkers that were self-ligated, the ligation product is cleaved with XhoI. Each catch linker has one half of an XhoI site at its termini; therefore, XhoI cleaves catch linkers ligated to themselves but will not cleave catch linkers ligated to most genomic DNA fragments. The linked DNA is in a form that can be amplified by PCR™ using the catch oligomers as primers. The DNA can then be selected via binding to a protein or nucleic acid and then recovered. The small amount of DNA fragments specifically bound can be amplified using PCR™. The steps of selection and amplification may be repeated as often as necessary to achieve the desired purity. Although 0.5 ng of starting DNA was amplified 5000-fold, Kinzler and Vogelstein (1989) did report a bias toward the amplification of smaller fragments.

Lone Linker PCR™

Because of the inefficiency of the conventional catch linkers due to self-hybridization of two complementary primers, asymmetrical linkers for the primers were designed (Ko et al., 1990). The sequences of the catch linker oligonucleotides (Kinzler and Vogelstein, 1989) were used with the exception of a deleted 3 base pair sequence from the 3′-end of one strand. This “lone-linker” has both a non-palindromic protruding end and a blunt end, thus preventing multimerization of linkers. Moreover, as the orientation of the linker was defined, a single primer was sufficient for amplification. After digestion with four-base cutting enzyme, the lone linkers were ligated. Lone-linker PCR™ (LL-PCR™) produces fragments ranging from a 100 bases to ˜2 kb that were reported to be amplified with similar efficiency.

Linker Adapter PCR™

The limitations of IRS-PCR™ (discussed below) are abated to some extent using the linker adapter technique (LA-PCR™) (Lüdecke et al., 1989; Saunders et al., 1989; Kao and Yu, 1991). This technique amplifies unknown restricted DNA fragments with the assistance of ligated duplex oligonucleotides (linker adapters). DNA is commonly digested with a frequently cutting restriction enzyme such as RsaI yielding fragments that are on average 500 bp in length. After ligation, PCR™ can be performed by using primers complementary to the sequence of the adapters. Temperature conditions are selected to enhance annealing specifically to the complementary DNA sequences, which leads to the amplification of unknown sequences situated between the adapters. Post-amplification, the fragments are cloned. There should be little sequence selection bias with LA-PCR™ except on the basis of distance between restriction sites. Methods of LA-PCR™ overcome the hurdles of regional bias and species dependence common to IRS-PCR™. However, LA-PCR™ is technically more challenging than other whole genome amplification (WGA) methods.

A large number of band-specific microdissection libraries of human, mouse, and plant chromosomes have been established using LA-PCR (Chang et al., 1992; Wesley et al., 1990; Saunders et al., 1989; Vooijs et al., 1993; Hadano et al., 1991; Miyashita et al., 1994). PCR™ amplification of a microdissected region of a chromosome is conducted by digestion with a restriction enzyme (e.g., Sau3A, MboI) to generate a number of short fragments, which are ligated to linker-adapter oligonucleotides that provide priming sites for PCR™ amplification (Saunders et al., 1989). Two oligonucleotides, a 20-mer and a 24-mer carrying a 5′ overhang that was phosphorylated with T4 polynucleotide kinase and complementary to the end created by the restriction enzyme, were mixed in equimolar amounts, and allowed to anneal. Following this amplification, as much as 1 μg of DNA can be amplified from as little as one band dissected from a polytene chromosome (Saunders et al., 1989; Johnson, 1990). Ligation of a linker-adapter to each end of the chromosomal restriction fragment provides the primer-binding site necessary for in vitro semiconservative DNA replication. Other applications of this technology include the amplification of a single flow-sorted mouse chromosome 11 and use of the resulting DNA library as a probe in chromosome painting (Miyashita et al., 1994), and the amplification of DNA of a single flow-sorted chromosome (VanDeanter et al., 1994).

A different adapter used in PCR™ is the Vectorette (Riley et al., 1990). This technique is largely used for the isolation of terminal sequences from yeast artificial chromosomes (YAC) (Kleyn et al., 1993; Naylor et al., 1993; Valdes et al., 1994). Vectorette is a synthetic oligonucleotide duplex containing an overhang complementary to the overhang generated by a restriction enzyme. The duplex contains a region of non-complementarity as a primer-binding site. After ligation of digested YACs and a Vectorette unit, amplification is performed between primers identical to Vectorette and primers derived from the yeast vector. Products will only be generated if in the first PCR™ cycle synthesis has originated from the yeast vector primer, thus producing products starting from the termini of the YAC inserts.

Single Cell Comparative Genomic Hybridization

A method allowing the comprehensive analysis of the entire genome on a single cell level has been developed and termed single cell comparative genomic hybridization (SCOMP) (Klein et al., 1999; WO 00/17390). Genomic DNA from a single cell is fragmented with a four base cutter, such as MseI, giving an expected average length of 256 bp (4⁴) based on the premise that the four bases are evenly distributed. Ligation mediated PCR™ was utilized to amplify the digested restriction fragments. Briefly, two primers (5′-AGTGGGATTCCGCATGCTAGT-3′; SEQ ID NO:3) and (5′-TAACTAGCATGC-3′; SEQ ID NO:4) were annealed to each other to create an adaptor with two 5′ overhangs. The 5′ overhang resulting from the shorter oligo is complementary to the ends of the DNA fragments produced by MseI cleavage. The adaptor was ligated to the digested fragments using T4 DNA ligase. Only the longer primer was ligated to the DNA fragments as the shorter primer did not have the 5′ phosphate necessary for ligation. Following ligation, the second primer was removed via denaturation, and the first primer remained ligated to the digested DNA fragments. The resulting 5′ overhangs were filled in by the addition of DNA polymerase. The resulting mixture was then amplified by PCR™ using the longer primer.

As this method is reliant on restriction digests to fragment the genomic DNA, it is dependent on the distribution of restriction sites in the DNA. Very small and very long restriction fragments will not be effectively amplified, resulting in a biased amplification. The average fragment length of 256 bp generated by MseI cleavage will result in a large number of fragments that are too short to amplify.

Random Primed PCR™

Random primed PCR™ based mechanisms have been utilized to amplify all or part of a genome. The amplification of complete pools of DNA, termed known amplification (Lüdecke et al., 1989) or general amplification (Telenius et al., 1992), can be achieved by different means. Common to all approaches is the capability of the PCR™ system to unanimously amplify DNA fragments in the reaction mixture without preference for specific DNA sequences. The structure of primers used for whole genome PCR™ is described as totally degenerate (i.e., all nucleotides are termed N, N=A, T, G, C), partially degenerate (i.e., several nucleotides are termed N) or non-degenerate (i.e., all positions exhibit defined nucleotides). The major drawback of all of these methods is the inability to prime all regions with similar efficiency. This usually results in very uneven amplification of different loci which increases the difficulty in genotyping the samples and prevents the analysis of copy number and other important changes that occur during disease progression. The Random primed PCR™ methods that have been utilized are described below.

Priming Authorizing Random Mismatches PCR™

One whole genome PCR™ method using non-degenerate primers is Priming Authorizing Random Mismatches-PCR™ (PARM-PCR™), which uses specific primers and unspecific annealing conditions resulting in a random hybridization of primers leading to universal amplification (Milan et al., 1993). Annealing temperatures are reduced to 30° C. for the first two cycles and raised to 60° C. in subsequent cycles to specifically amplify the generated DNA fragments. This method has been used to universally amplify flow sorted porcine chromosomes for identification via fluorescent in situ hybridization (FISH) (Milan et al., 1993). A similar technique was also used to generate chromosome DNA clones from microdissected DNA (Hadano et al., 1991). In this method, a 22-mer primer unique in sequence, which randomly primes and amplifies any target DNA, was utilized. The primer exhibited recognition sites for three restriction enzymes. Thermocycling was done in three stages: stage one had an annealing temperature of 22° C. for 120 minutes, and stages two and three were conducted under stringent annealing conditions.

Interspersed Repetitive Sequence PCR™

As used for the general amplification of DNA, interspersed repetitive sequence PCR™ (IRS-PCR™) uses non-degenerate primers that are based on repetitive sequences within the genome. This allows for amplification of segments between suitable positioned repeats and has been used to create human chromosome- and region-specific libraries (Nelson et al., 1989). IRS-PCR™ is also termed Alu element mediated-PCR™ (ALU-PCR™), which uses primers based on the most conserved regions of the Alu repeat family and allows the amplification of fragments flanked by these sequences (Nelson et al., 1989). A major disadvantage of IRS-PCR™ is that abundant repetitive sequences like the Alu family are not uniformly distributed throughout the human genome, but preferentially found in certain areas (e.g., the light bands of human chromosomes) (Korenberg and Rykowski, 1988). Thus, IRS-PCR™ results in a bias toward such regions and a lack of amplification of less represented areas. Moreover, this technique is dependent on the knowledge of the presence of abundant repeat families in the genome of interest.

Degenerate Oligonucleotide Primed PCR™

Degenerate oligonucleotide-primed PCR™ (DOP-PCR™) was developed using partially degenerate primers, thus providing a more general amplification technique than IRS-PCR™ (Wesley et al., 1990; Telenius, 1992). A system was described using non-specific primers (5′-TTGCGGCCGCATNNNNTTC-3′; SEQ ID NO:5) showing complete degeneration at positions 4, 5, 6, and 7 from the 3′ end (Wesley et al., 1990). The three specific bases at the 3′end are statistically expected to hybridize every 64 (43) bases, thus the last seven bases will match due to the partial degeneration of the primer. The first cycles of amplification are conducted at a low annealing temperature (30° C.), allowing sufficient priming to initiate DNA synthesis at frequent intervals along the template. The defined sequence at the 3′ end of the primer tends to separate initiation sites, thus increasing product size. As the PCR™ product molecules all contain a common specific 5′ sequence, the annealing temperature is raised to 56° C. after the first eight cycles. The system was developed to non-specifically amplify microdissected chromosomal DNA from Drosophila, replacing the microcloning system of Lüdecke et al. (1989) described above.

The term DOP-PCR™ was introduced by Telenius et al. (1992) who developed the method for genome mapping research using flow sorted chromosomes. A single primer is used in DOP-PCR™ as used by Wesley et al. (1990). The primer (5′-CCGACTCGACNNNNNNATGTGG-3′; SEQ ID NO:6) shows six specific bases on the 3′-end, a degenerate part with 6 bases in the middle and a specific region with a rare restriction site at the 5′-end. Amplification occurs in two stages. Stage one encompasses the low temperature cycles. In the first cycle, the 3′-end of the primers hybridize to multiple sites of the target DNA initiated by the low annealing temperature. In the second cycle, a complementary sequence is generated according to the sequence of the primer. In stage two, primer annealing is performed at a temperature restricting all non-specific hybridization. Up to 10 low temperature cycles are performed to generate sufficient primer binding sites. Up to 40 high temperature cycles are added to specifically amplify the prevailing target fragments.

DOP-PCR™ is based on the principle of priming from short sequences specified by the 3′-end of partially degenerate oligonucleotides used during initial low annealing temperature cycles of the PCR™ protocol. As these short sequences occur frequently, amplification of target DNA proceeds at multiple loci simultaneously. DOP-PCR™ is applicable to the generation of libraries containing high levels of single copy sequences, provided uncontaminated DNA in a substantial amount is obtainable (e.g., flow-sorted chromosomes). This method has been applied to less than one nanogram of starting genomic DNA (Cheung and Nelson, 1996).

Advantages of DOP-PCR™ in comparison to systems of totally degenerate primers are the higher efficiency of amplification, reduced chances for non-specific primer-primer binding and the availability of a restriction site at the 5′ end for further molecular manipulations. However, DOP-PCR™ does not claim to replicate the target DNA in its entirety (Cheung and Nelson, 1996). Moreover, as relatively short products are generated, specific amplification of fragments up to approximately 500 bp in length are produced (Telenius et al., 1992; Cheung and Nelson, 1996; Wells et al., 1999; Sanchez-Cespedes et al., 1998; Cheng et al., 1998).

In light of these limitations, a method has been described that produces long DOP-PCR™ products ranging from 0.5 to 7 kb in size, allowing the amplification of long sequence targets in subsequent PCR™ (long DOP-PCR™) (Buchanan et al., 2000). However, long DOP-PCR™ utilizes 200 ng of genomic DNA, which is more DNA than most application will have available. Subsequently, a method was described that generates long amplification products from picogram quantities of genomic DNA, termed long products from low DNA quantities DOP-PCR™ (LL-DOP-PCR™) (Kittler et al., 2002). This method achieves this by the 3′-5′ exonuclease proofreading activity of DNA polymerase Pwo and an increased annealing and extension time during DOP-PCR™, which are necessary steps to generate longer products. Although an improvement in success rate was demonstrated in comparison with other DOP-PCR™ methods, this method did have a 15.3% failure rate due to complete locus dropout for the majority of the failures, and sporadic locus dropout and allele dropout for the remaining genotype failures. There was a significant deviation from random expectations for the occurrence of failures across loci, thus indicating a locus-dependent effect on whole genome coverage.

Sequence Independent PCR™

Another approach using degenerate primers is described by Bohlander et al., (1992), called sequence-independent DNA amplification (SIA). In contrast to DOP-PCR™, SIA incorporates a nested DOP-primer system. The first primer (5′-TGGTAGCTCTTGATCANNNNN-3′; SEQ ID NO:7) consists of a five base random 3′-segment and a specific 16 base segment at the 5′ end containing a restriction enzyme site. Stage one of PCR™ starts with 97° C. for denaturation, followed by cooling down to 4° C., causing primers to anneal to multiple random sites, and then heating to 37° C. A T7 DNA polymerase is used. In the second low-temperature cycle, primers anneal to products of the first round. In the second stage of PCR™, a second primer (5′-AGAGTTGGTAGCTCTTGATC-3′; SEQ ID NO:8) is used that contains, at the 3′ end, the 15 5′-end bases of primer A. Five cycles are performed with this primer at an intermediate annealing temperature of 42° C. An additional 33 cycles are performed at a specific annealing temperature of 56° C. Products of SIA range from 200 bp to 800 bp.

Primer-Extension Preamplification

Primer-extension preamplification (PEP) is a method that uses totally degenerate primers to achieve universal amplification of the genome (Zhang et al., 1992). PEP uses a random mixture of 15-base fully degenerate oligonucleotides as primers, thus any one of the four possible bases could be present at each position. Theoretically, the primer is composed of a mixture of 4×10⁹ different oligonucleotide sequences. This leads to amplification of DNA sequences from randomly distributed sites. In each of the 50 cycles, the template is first denatured at 92° C. Subsequently, primers are allowed to anneal at a low temperature (37° C.), which is then continuously increased to 55° C. and held for another four minutes for polymerase extension.

A method of improved PEP (I-PEP) was developed to enhance the efficiency of PEP, primarily for the investigation of tumors from tissue sections used in routine pathology to reliably perform multiple microsatellite and sequencing studies with a single or few cells (Dietmaier et al., 1999). I-PEP differs from PEP (Zhang et al., 1992) in cell lysis approaches, improved thermal cycle conditions, and the addition of a higher fidelity polymerase. Specifically, cell lysis is performed in EL buffer, Taq polymerase is mixed with proofreading Pwo polymerase, and an additional elongation step at 68° C. for 30 seconds is performed before the denaturation step at 94° C. This method was more efficient than PEP and DOP-PCR™ in amplification of DNA from one cell and five cells.

Both DOP-PCR™ and PEP have been used successfully as precursors to a variety of genetic tests and assays. These techniques are integral to the fields of forensics and genetic disease diagnostics where DNA quantities are limited. However, neither technique claims to replicate DNA in its entirety (Cheung and Nelson, 1996) or provide complete coverage of particular loci (Paunio et al., 1996). These techniques produce an amplified source for genotyping or marker identification. The products produced by these methods are consistently short (<3 kb) and, therefore, cannot be used in many applications (Telenius et al., 1992). Moreover, numerous tests are required to investigate a few markers or loci.

Tagged PCR™

Tagged PCR™ (T-PCR™) was developed to increase the amplification efficiency of PEP in order to amplify efficiently from small quantities of DNA samples with sizes ranging from 400 bp to 1.6 kb (Grothues et al., 1993). T-PCR™ is a two-step strategy, which uses for the first few low-stringent cycles a primer with a constant 17 base sequence at the 5′ end and a tagged random primer containing nine to 15 random bases at the 3′ end. In the first PCR™ step, the tagged random primer is used to generate products with tagged primer sequences at both ends, which is achieved by using a low annealing temperature. The unincorporated primers are then removed and amplification is carried out with a second primer containing only the constant 5′ sequence of the tagged primer, under high-stringency conditions for exponential amplification. This method is more labor intensive than other methods due to the requirement for removal of unincorporated degenerate primers, which can also result in the loss of sample material. This is critical when working with subnanogram quantities of DNA template. The unavoidable loss of template during the purification steps can also affect the coverage of T-PCR™. Moreover, tagged primers with 12 or more random bases could generate non-specific products resulting from primer-primer extensions or less efficient elimination of longer primers during the filtration step.

Tagged Random Hexamer Amplification

Based on problems related to T-PCR™, tagged random hexamer amplification (TRHA) was developed on the premise that it would be advantageous to use a tagged random primer with fewer random bases (Wong et al., 1996). In TRHA, the first step is to produce a size distributed population of DNA molecules from a pNL1 plasmid. This was done via a random synthesis reaction using Klenow fragment and a random hexamer primer tagged with a T7 primer sequence at the 5′-end (T7-dN₆, 5′-GTAATACGACTCACTATAGGGCNNNNNN-3′; SEQ ID NO:9). Klenow-synthesized molecules (size range 28 bp-<23 kb) were then amplified with T7 primer (5′-GTAATACGACTCACTATAGGGC-3′; SEQ ID NO:10). Examination of bias indicated that only 76% of the original DNA template was preferentially amplified and represented in the TRHA products.

Strand Displacement Mediated Amplification

Strand displacement mediated amplification methods rely on DNA polymerases that have a strong ability to displace DNA strands that would block other polymerases from continuing to extend DNA fragments. This displacement reaction results in branched molecules that can also be primed and extended. Use of random primers to initiate DNA polymerization allows priming at multiple points of the parent molecule, as well as on the displaced DNA strands. A cascading series of priming, polymerization, and strand displacement results in a highly branched molecule resulting in amplification of the majority of the sequences. The advantages of this type of system include isothermal reactions, minimal manipulation of the starting DNA, and the production of large amounts of amplified products. The drawbacks to these methods are the requirement that the starting material consist of high MW DNA, the difficulty in priming/extending equally over all regions, and the tendency to produce non-sense DNA in the absence of template. Brief descriptions of the major strand-displacement mediated amplification methods are documented below.

Rolling Circle Amplification

The isothermal technique of rolling circle amplification (RCA) has been developed for amplifying large circular DNA templates such as plasmid and bacteriophage DNA (Dean et al., 2001). Using φ29 DNA polymerase, which synthesizes DNA strands 70 kb in length using random exonuclease-resistant hexamer primers, DNA was amplified in a 30° C. isothermal reaction. Secondary priming events occur on the displaced product DNA strands, resulting in amplification via strand displacement.

In this technique, two sets of primers are used. The first set of primers each have a portion complementary to nucleotide sequences flanking one side of a target nucleotide sequence and primers in the second set of primers each have a portion complementary to nucleotide sequences flanking the other side of the target nucleotide sequence. The primers in the first set are complementary to one strand of the nucleic acid molecule containing the target nucleotide sequence, and the primers in the left set are complementary to the opposite strand. The 5′ end of primers in both sets is distal to the nucleic acid sequence of interest when the primers are hybridized to the flanking sequences in the nucleic acid molecule. Ideally, each member of each set has a portion complementary to a separate, and non-overlapping, nucleotide sequence flanking the target nucleotide sequence. Amplification proceeds by replication initiated at each priming site and continues through the target nucleic acid sequence. A key feature of this method is the displacement of intervening primers during replication. Another round of priming and replication commences after the nucleic acid strands elongated from the first set of primers reaches the region of the nucleic acid molecule to which the second set of primers hybridizes, and vice versa. This allows multiples copies of a nested set of the target nucleic acid sequence to be synthesized.

Multiple Displacement Amplification

The principles of RCA have been extended to WGA in a technique called multiple displacement amplification (MDA) (Dean et al., 2002; U.S. Pat. No. 6,280,949 B1). In this technique, a random set of primers is used to randomly prime a sample of genomic DNA. By selecting a sufficiently large set of primers of random or partially random sequence, the primers in the set will be collectively, and randomly, complementary to nucleic acid sequences distributed throughout nucleic acids in the sample. Amplification proceeds by replication with a highly processive polymerase, φ29 DNA polymerase, initiating at each primer and continuing until spontaneous termination. Displacement of intervening primers during replication by the polymerase allows multiple overlapping copies of the entire genome to be synthesized.

The use of random primers to universally amplify genomic DNA is based on the assumption that random primers equally prime over the entire genome, thus allowing representative amplification. Although the primers themselves are random, the location of primer hybridization in the genome is not random, as different primers have unique sequences and thus different characteristics (such as different melting temperatures). As random primers do not equally prime everywhere over the entire genome, amplification is not completely representative of the starting material. Such protocols are useful in studying specific loci, but the result of random-primed amplification products is not representative of the starting material (e.g., the entire genome). Therefore, there is a need for a technique to prepare the genomic DNA to use with non-random primers that will result in representative amplification of the starting material.

Cell Immortalization

Cell immortalization methods for amplifying large amounts of DNA rely on the ability of cells to faithfully replicate their own DNA during cell division. This is a commonly practiced method for producing large amounts of DNA from important sources for research and commercial use. The advantages of this method are the relative ease of preparing DNA, the high fidelity of the cells in replicating their DNA, and the maintenance of genetic and epigenetic information in the isolated DNA. The drawbacks of this method are the high cost, labor intensive, and slow methods necessary for generating large amounts of DNA from cells. The characteristics, advantages and problems with utilizing cell immortalization techniques for amplifying DNA are illustrated in the following section.

Normal human somatic cells have a limited life span and enter senescence after a limited number of cell divisions (Hayflick and Moorhead, 1961; Hayflick 1965; Martin et al., 1970). At senescence, cells are viable but no longer divide. This limit on cell proliferation represents an obstacle to the study of normal human cells, especially since many rounds of cell division are required to share cells between laboratories, and to produce the large quantities of cells required for biochemical analysis, genetic manipulations, and/or genetic screens. This limitation is of particular concern for the study of rare hereditary human diseases, since the volume of the biological samples collected (biopsies or blood) is usually small and contains a limited number of cells.

The establishment of permanent cell lines is one way to circumvent this lack of critical material. Some tumor cells yield cultures with unlimited growth potential, and in vitro transformation with oncogenes or carcinogens have proven a successful means to establish permanent fibroblast and lymphoblast cell lines. Such cell lines have been valuable in the analysis of mammalian biochemistry and the identification of disease-related genes. However, such transformed cells typically exhibit significant alterations in physiological and biological properties. Most notably, these cells are associated with aneuploidy, spontaneous hypermutability, loss of contact inhibition and alterations in biochemical functions related to cell cycle checkpoints. Those cellular properties that differ from their normal counterparts pose significant limitations to the analysis of many cellular functions, in particular those related to genomic integrity and the study of human chromosome instability syndromes.

Recent advances have shown that the onset of replicative senescence is controlled by the shortening of the telomeres that occurs each time normal human cells divide (Allsopp et al., 1992; Allsopp et al., 1995; Bodnar et al., 1998; Vaziri and Benchimol, 1998). This loss of telomeric DNA is a consequence of the inability of DNA polymerase alpha to fully replicate the ends of linear DNA molecules (Watson, 1972; Olovnikov, 1973). It has been proposed that senescence is induced when the shortest one or two telomeres can no longer be protected by telomere-binding proteins, and thus is recognized as a double-stranded (ds) DNA break. In cells with functional checkpoints, the introduction of dsDNA breaks leads to the activation of p53 and of the p16/pRB checkpoint and to a growth arrest state that mimics senescence (Vaziri and Benchimol, 1996; Di Leonardo et al., 1994; Robles and Adami, 1998). Cell cycle progression in senescent cells is also blocked by the same two mechanisms (Bond et al., 1996; Hara et al., 1996; Shay et al., 1991). This block can be overcome by viral oncogenes, such as SV40 large T antigen, that can inactivate both p53 and pRB. Cells that express SV40 large T antigen escape senescence but continue to lose telomeric repeats during their extended life span. These cells are not yet immortal, and terminal telomere shortening eventually causes the cells to reach a second non-proliferative stage termed ‘crisis’ (Counter et al., 1992; Wright and Shay; 1992). Escape from crisis is a very rare event (1 in 10⁷) usually accompanied by the reactivation of telomerase (Shay et al., 1993).

Telomerase is a specialized cellular reverse transcriptase that can compensate for the erosion of telomeres by synthesizing new telomeric DNA. The activity of telomerase is present in certain germline cells but is repressed during development in most somatic tissues, with the exception of proliferative descendants of stem cells such as those in the skin, intestine and blood (Ulaner and Giudice, 1997; Wright et al., 1996; Yui et al., 1998; Ramirez et al., 1997; Hiyama et al., 1996). The telomerase enzyme is a ribonuclear protein composed of at least two subunits; an integral RNA that serves as a template for the synthesis of telomeric repeats (hTR) and a protein (hTERT) that has reverse transcriptase activity. The RNA component (hTR) is ubiquitous in human cells, but the presence of the mRNA encoding hTERT is restricted to cells with telomerase activity. The forced expression of exogenous hTERT in normal human cells is sufficient to produce telomerase activity in these cells and prevent the erosion of telomeres and circumvent the induction of both senescence and crisis (Bodnar et al., 1998; Vaziri and Benchimol, 1998). Recent studies have shown that telomerase can immortalize a variety of cell types. Cells immortalized with hTERT have normal cell cycle controls, functional p53 and pRB checkpoints, are contact inhibited, are anchorage dependent, require growth factors for proliferation, and possess a normal karyotype (Morales et al., 1999; Jiang et al., 1999).

Patents and Patent Applications Related to Whole Genome Amplification

Thus, the related art provides a variety of techniques for whole genome amplification, although there remains a need in the art for methods and compositions amenable to non-biased high throughput library generation and/or preparation of DNA molecules. For example, Japan Patent No. JP8173164A2 describes a method of preparing DNA by sorting-out PCR™ amplification in the absence of cloning, fragmenting a double-stranded DNA, ligating a known-sequence oligomer to the cut end, and amplifying the resultant DNA fragment with a primer having the sorting-out sequence complementary to the oligomer. The sorting-out sequences consist of a fluorescent label and one to four bases at 5′ and 3′termini to amplify the number of copies of the DNA fragment.

U.S. Pat. No. 6,107,023 describes a method of isolating duplex DNA fragments which are unique to one of two fragment mixtures, i.e., fragments which are present in a mixture of duplex DNA fragments derived from a positive source, but absent from a fragment mixture derived from a negative source. In practicing the method, double-strand linkers are attached to each of the fragment mixtures, and the number of fragments in each mixture is amplified by successively repeating the steps of (i) denaturing the fragments to produce single fragment strands; (ii) hybridizing the single strands with a primer whose sequence is complementary to the linker region at one end of each strand, to form strand/primer complexes; and (iii) converting the strand/primer complexes to double-stranded fragments in the presence of polymerase and deoxynucleotides. After the desired fragment amplification is achieved, the two fragment mixtures are denatured, then hybridized under conditions in which the linker regions associated with the two mixtures do not hybridize. DNA species which are unique to the positive-source mixture, i.e., which are not hybridized with DNA fragment strands from the negative-source mixture, are then selectively isolated.

U.S. Pat. No. 6,114,149 regards a method of amplifying a mixture of different-sequence DNA fragments that may be formed from RNA transcription, or derived from genomic single- or double-stranded DNA fragments. The fragments are treated with terminal deoxynucleotide transferase and a selected deoxynucleotide, to form a homopolymer tail at the 3′ end of the anti-sense strands, and the sense strands are provided with a common 3′-end sequence. The fragments are mixed with a homopolymer primer that is homologous to the homopolymer tail of the anti-sense strands, and a defined-sequence primer which is homologous to the sense-strand common 3′-end sequence, with repeated cycles of fragment denaturation, annealing, and polymerization, to amplify the fragments. In one embodiment, the defined-sequence and homopolymer primers are the same, i.e., only one primer is used. The primers may contain selected restriction-site sequences, to provide directional restriction sites at the ends of the amplified fragments.

U.S. Patent Application Publication US 2003/0013671 relates to methods and compositions regarding a genomic DNA library that substantially maintains copy numbers of a set of sequences and an abundance ratio of 1 to 5 as defined by the size ratio of the maximum size to the minimum size of fragmented DNA. In particular methods, genomic DNA is randomly fragmented, adaptors are ligated, and the fragments are amplified.

In contrast to other methods in the art, the present invention provides a variety of new ways of preparing DNA templates based on ligation mediated PCR™, particularly for whole genome amplification, and preferentially in a manner representative of a native genome.

### SUMMARY OF THE INVENTION

The present invention regards the amplification of a whole genome, including various methods and compositions to achieve that goal. In a specific embodiment, a whole genome is amplified from a single cell, and in other embodiments the whole genome is amplified from a plurality of cells or from a cell-free state.

In a particular aspect of the present invention, the invention is directed to methods for the amplification of substantially the entire genome without loss of representation of specific sites (herein defined as “whole genome amplification”). In a specific embodiment, whole genome amplification comprises simultaneous amplification of substantially all fragments of a genomic library. In a further specific embodiment, “substantially entire” or “substantially all” refers to about 80%, about 85%, about 90%, about 95%, about 97%, about 99%, or 100% of all sequences in a genome. A skilled artisan recognizes that amplification of the whole genome will, in some embodiments, comprise non-equivalent amplification of particular sequences over others, although the relative difference in such amplification is not considerable.

In one method, genomic DNA is fragmented, such as mechanically, to generate double stranded DNA fragments with a size distribution of about 500 bp to about 3 kb. Following fragmentation, the 3′ ends of the DNA are repaired and extended to produce attachable ends, such as by producing blunt-end products. In a specific embodiment, the term “repaired” refers to the excision of at least one base, such as a defective base, on an end of at least one DNA molecule, followed by polymerization. In a specific embodiment, the distal-most excised base lacks a 3′ hydroxyl group prior to repair. In another specific embodiment, the term “repaired” may be used interchangeably with the term “polished”.

In these particular methods, an adaptor comprising a known sequence is ligated to the 5′ end of each end of the DNA duplex to produce a single strand 5′ overhang with known sequence. Subsequently, the ligated DNA duplex is extended by polymerase to fill in the 5′ overhang and generate a double stranded adaptor site. The resulting molecules are amplified using a primer comprising known sequence, resulting in at least about several thousand-fold amplification of the entire genome without bias. The products of this amplification can be re-amplified additional times, resulting in amplification in excess of about several million fold.

The present invention utilizes double stranded or single stranded DNA. That is, single stranded DNA is obtained and processed according to the methods described herein. Embodiments well-suited to ssDNA-related methods include the thermal fragmentation methods described herein, for example. In other embodiments, double stranded DNA is obtained and processed according to methods described herein, and embodiments well-suited to these dsDNA-related methods include the exemplary mechanical hydroshear fragmentation and/or enzymatic fragmentation methods.

In yet another aspect of the present invention, there are novel methods of converting double-stranded DNA into a randomly fragmented, end-linkered library in a single reaction, in a single tube or well, and/or in a single system. The method depends on the development of reaction buffer that can support both endonuclease cleavage and ligase activity. Special linkers are designed that can be attached to all possible ends of endonuclease cleavage but that cannot self-ligate. In a single reaction, in a single tube or well, and/or in a single system, double-stranded DNA, endonuclease, ligase, and linkers, for example, are incubated. By effectively modulating cleavage and ligation kinetics, end-linkered fragments of a desired average size can be obtained. In a specific embodiment, the method is employed for whole genome amplification.

Thus, in this aspect of the disclosure, the invention provides a method for converting DNA into libraries that overcomes many of the above-mentioned problems associated with the prior art. Specifically, in this embodiment there is a one-step method for library construction that does not require sequential enzymatic steps, DNA purification steps, or even an intermediate reagent addition step, which renders the invention particularly well-suited to high throughput library generation. The invention also allows for multiple libraries of different average fragment sizes to be generated from a single reaction. Specific objects of this embodiment are to provide a reaction buffer that can support both endonuclease cleavage and ligation, the design of double-stranded linkers that can be attached to fragment ends, and/or reaction conditions to obtain an end-linkered library. In a specific embodiment, the method comprises using a buffer for a single-step reaction wherein the reaction comprises endonuclease cleavage and ligase activity. In another specific embodiment, the method consists essentially of preparing a DNA molecule using a buffer for a single-step reaction comprising both endonuclease cleavage and ligase activity.

In one embodiment of the present invention, there is a method of preparing a DNA molecule, comprising obtaining at least one DNA molecule; randomly fragmenting the DNA molecule to produce DNA fragments; modifying the ends of the DNA fragments (which can be single stranded or double stranded) to comprise double stranded ends; attaching an adaptor having a known sequence to one strand at both ends of a plurality of the DNA fragments to produce a plurality of adaptor-linked fragments, wherein the 5′ end of the DNA is attached to a nonblocked 3′ end of the adaptor, leaving a nick at the juxtaposed 3′ end of the DNA and 5′ end of the adaptor; extending the 3′ end of the nick; and amplifying a plurality of the adaptor-linked fragments.

In a specific embodiment, the polishing step, wherein the ends of DNA fragments are rendered blunt or rendered with at least one approximately one- or two-nucleotide overhang, is circumvented. In a particular aspect of the invention, this occurs by determining the nature of the ends of the fragments in the population and then applying a proportionate amount of appropriate adaptors for ligation to the ends. This determination occurs, for example, empirically for each sample. In a specific embodiment, adaptor(s) are tested separately and, in alternative embodiments, in combination with others, for ligatability to the DNA ends. A ratio of different adaptors appropriate for the population is identified, for example in a pilot study, and this identified ratio, or a ratio approximate to the identified ratio, is then utilized to prepare a larger population of DNA molecules. This may be tested, for example, such as by assaying for the ability to utilize the adaptors as priming sites for polymerase chain reaction.

In a particular aspect of the invention, there is a method of preparing a DNA molecule, comprising obtaining at least one DNA molecule, such as a genome, for example; randomly fragmenting the DNA molecule to produce DNA fragments; modifying the ends of the DNA fragments to provide attachable ends; attaching an adaptor having at least one known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor; extending the 3′ end of the modified DNA from the nick site; and amplifying a plurality of the adaptor-linked fragments.

In specific embodiments, a first adaptor having a first known sequence (or more) is attached to a first end of the modified DNA fragments, and a second adaptor having a second known sequence (or more) is attached to a second end of the modified DNA fragments. In more specific embodiments, the first and second known sequences are nonidentical. In other specific embodiments, the first known sequence and the second known sequence comprise sequences (for example, by being designed as such) that do not substantially interact. For example, the first and second known sequences may comprise nucleotides that are non-self-complementary and noncomplementary to each other, such as by comprising nucleotides that are incapable of forming Watson-Crick base pairs. A skilled artisan recognizes that such a design on the adaptors facilitates avoiding primer dimer formation during, for example, amplification reactions using primers complementary to the first and second adaptors. In specific embodiments, the adaptor comprises at least one of the following features: absence of a 5′ phosphate group; a 5′ overhang; or a blocked 3′ base. The 5′ overhang may comprise about 5 to about 100 bases.

The modifying step may further be defined as modifying the ends of the DNA fragments to comprise blunt double stranded ends or further defined as modifying the ends of the DNA fragments to comprise an overhang of at least 1 nucleotide.

Randomly fragmenting the DNA molecule may comprise mechanical fragmentation, such as, for example, hydrodynamic shearing, sonication, nebulization, or a combination thereof. Randomly fragmenting the DNA molecule may also comprise chemical fragmentation, such as by acid catalytic hydrolysis, alkaline catalytic hydrolysis, hydrolysis by metal ions, hydroxyl radicals, irradiation, heating, or a combination thereof. Randomly fragmenting the DNA molecule may also comprise enzymatic fragmentation, such as by DNAse I digestion or Cvi JI restriction enzyme digestion.

Any modifying step of the present invention may comprise repair of at least one 3′ end of the DNA fragment, such as, for example, by subjecting the DNA fragment to 3′ exonuclease activity, 5′-3′ polymerase activity, or both. In a particular embodiment, both of the 3′ exonuclease activity and the 5′-3′ polymerase activity are comprised in the same enzyme, such as Klenow, T4 DNA polymerase, or a mixture thereof. In a specific embodiment, the 3′ exonuclease activity comprises Exonuclease III activity and the 3′ polymerase activity comprises T4 DNA polymerase activity. Following the subjecting step, the DNA fragments are subjected to Klenow, T4 DNA polymerase, or both. The DNA fragments may comprise a plurality of ssDNA molecules and the modifying step may be further defined as subjecting the ssDNA molecules to a plurality of random primers and DNA polymerase activity, under conditions wherein the blunt double stranded fragments are thereby generated.

In a specific embodiment, the random primers further comprise a known sequence at their 5, end. In another specific embodiment, at least one ssDNA molecule comprises a blocked 3, end and the modifying step is further defined as subjecting the ssDNA to 3′-5′ exonuclease activity.

Random primers utilized in the invention may be pentamers, hexamers, septamers, or octamers, and they may be phosphorylated at the 5′ end. Furthermore, the random primers may be comprised of at least one base analog, at least one backbone analog, or both. The DNA polymerase activity and the 3′-5′ exonuclease activity are comprised in the same enzyme, which may be a non strand-displacing polymerase, such as T4 DNA polymerase, or a strand-displacing polymerase, such as Klenow or DNA polymerase I. In a specific embodiment, the polymerase comprises nick translation activity, such as Klenow, T4 DNA polymerase, or DNA polymerase I, or a mixture thereof. In a specific embodiment, the modifying step and the attaching step occurs concomitantly.

In particular embodiments, enzymatic fragmentation occurs in the presence of Mn²⁺ and the modifying step is further defined as subjecting the DNA fragments to 3, exonuclease activity, 5′-3′ polymerase activity, or both. In another particular embodiment, the enzymatic fragmentation occurs in the presence of Mg²⁺ and the modifying step is further defined as subjecting the DNA fragments to random primers, 5′-3′ polymerase activity and 3′-5′ exonuclease activity.

In specific embodiments of the present invention, the attaching step is further defined as subjecting the DNA fragments to a blunt end adaptor, a 5′ overhang adaptor, a 3, overhang adaptor, or a mixture thereof.

Adaptors of the present invention may comprise at least one of the following features: absence of a 5′ phosphate group; a 5′ overhang; or a blocked 3′ base. In a specific embodiment, the 5′ overhang comprises about 5 to about 100 bases. The attachment may be by ligating the adaptor to the DNA fragment, such as through chemical ligation or enzymatic ligation, such as by T4 DNA ligase or topoisomerase I. Wherein topoisomerase I is utilized, the adaptor may be covalently attached to topoisomerase I at a 3′ thymidine overhang or a blunt end and the adaptor may comprise a sequence of 5′-CCCTT-3′.

In specific embodiments, DNA fragments are blunt ended and a 3′ adenosine is added to the blunt ended DNA fragments by polymerase.

The adaptors may also comprise a first primer and a second primer, wherein the first primer is greater in length than the second primer. Furthermore, the second primer may comprise a blocked 3′ end. Adaptors may comprise at least one blunt end. The 3, end of at least one primer is blocked. The adaptor may also comprise one oligonucleotide having two regions complementary to each other, wherein the regions are separated by a linker region. In some embodiments, when the two complementary regions are hybridized to each other to form a double-stranded region of the adaptor, the end of the double stranded region is a blunt end.

Adaptors of the present invention may be further defined as comprising a first adaptor having a first known sequence and further comprising a homopolymeric sequence. There are methods that further comprise the steps of digesting amplified adaptor-linked fragments to produce fragmented adaptor-linked fragments; attaching a second adaptor having a second known sequence to the ends of the fragmented adaptor-linked fragments to produce second adaptor-linked fragments; and amplifying the second adaptor-linked fragments with a primer complementary to the homopolymeric sequence and a primer complementary to the second known sequence. The adaptor may also be further defined as a first adaptor having a first known sequence. There may also be methods that further comprise the following steps: subjecting amplified adaptor-linked fragments to terminal deoxynucleotidyl transferase to generate a homopolymeric single-stranded tail on the amplified adaptor-linked fragments; digesting the homopolymeric tailed amplified adaptor-linked fragments; attaching a second adaptor having a second known sequence to the ends of the digested homopolymeric tailed amplified adaptor-linked fragments that do not comprise the homopolymeric tail, to produce second adaptor-linked fragments; and amplifying the second adaptor-linked fragments with a primer complementary to the homopolymeric sequence and a primer complementary to the second known sequence.

Homopolymeric sequences utilized in the present invention may be single stranded, such as a single stranded poly G or poly C. Also, the homopolymeric sequence may refer to a region of double stranded DNA wherein one strand of homopolymeric sequence comprises all of the same nucleotide, such as poly C, and the opposite strand of the double stranded region complementary thereto comprises the appropriate poly G.

Linker regions within adaptors may comprise a non-replicable organic chain of about 1 to about 50 atoms in length, and an example of a non-replicable organic chain is hexa ethylene glycol (HEG).

In particular embodiments, the extending step comprises subjecting the adaptor-linked fragments comprising the nick to a mixture comprising DNA polymerase; deoxynucleotide triphosphates; and suitable buffer, under conditions wherein polymerization occurs from the 3′ hydroxyl of the nick.

Methods described herein may further comprise heating the mixture, such as to a temperature of about 75° C. In this and other embodiments, the DNA polymerase is a thermophilic DNA polymerase, such as, for example, Taq polymerase. In particular embodiments, at least one deoxynucleotide triphosphate is labeled. Amplifying steps may comprise polymerase chain reaction that utilizes a primer complementary to a sequence of the adaptor. The primer may be labeled.

In particular embodiments, the DNA molecule is comprised in a cell or it may not be comprised in a cell. In specific embodiments, the DNA molecule is cell-free fetal DNA in maternal blood or is cell-free cancer DNA in blood. The obtaining step may further be defined as obtaining the at least one DNA molecule from blood, urine, sputum, feces, sweat, nipple aspirate, semen, a fixed tissue sample, cerebral spinal fluid, an immunoprecipitated chromatin, physically isolated chromatin, or a combination thereof.

Wherein the DNA molecule or molecules comprises genomic DNA, the genomic DNA may be from a bacterial genome, a viral genome, a fungal genome, a plant genome, an animal genome, such as a mammalian genome, or a genome of any extant or extinct species.

In another embodiment, there is a method of preparing a DNA molecule, comprising obtaining a plurality of DNA molecules, the DNA molecules defined as fragments from at least one larger DNA molecule; modifying the ends of the DNA fragments to provide attachable ends; attaching an adaptor having a known sequence and a nonblocked 3′ end to both ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor; extending the 3′ end of the modified DNA from the nick site; and amplifying a plurality of the adaptor-linked fragments.

In an additional embodiment of the present invention, there is a method of amplifying a genome, comprising the steps of obtaining at least one DNA molecule; randomly fragmenting the DNA molecule to produce DNA fragments; modifying the ends of the DNA fragments to provide attachable ends; attaching an adaptor having a known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor; extending the 3′ end of the modified DNA from the nick site; and amplifying a plurality of the adaptor-linked fragments.

In an additional embodiment, there is a method of generating a library, comprising the steps of obtaining at least one DNA molecule; randomly fragmenting the DNA molecule to produce DNA fragments; modifying the ends of the DNA fragments to provide attachable ends; attaching an adaptor having a known sequence and a nonblocked 3′ end to both ends of a plurality of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor; and extending the 3′ end of the modified DNA from the nick site. The method may further comprise amplifying a plurality of the adaptor-linked fragments.

In another embodiment, there is a method of preparing a DNA molecule, comprising: obtaining at least one DNA molecule; attaching a first adaptor having a first known sequence, a homopolymeric sequence and a nonblocked 3′ end to the ends of the DNA molecule to produce first adaptor-linked molecules, wherein the 5′ end of the DNA molecule is attached to the nonblocked 3′ end of the adaptor, leaving a nick site between the juxtaposed 3′ end of the DNA molecule and a 5′ end of the adaptor; digesting the adaptor-linked DNA molecules to produce DNA fragments; attaching a second adaptor having a second known sequence to the ends of the DNA fragments to produce second adaptor-linked fragments; and amplifying a plurality of the second adaptor-linked fragments.

In other embodiments, there is a method of preparing a DNA molecule, comprising obtaining a plurality of DNA molecules, said DNA molecules defined as fragments from at least one larger DNA molecule; modifying the ends of the DNA fragments to provide attachable ends; attaching an adaptor having a known sequence and a nonblocked 3′ end to both ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor; extending the 3′ end of the modified DNA from the nick site; and amplifying a plurality of the adaptor-linked fragments. The at least one larger DNA molecule may comprise genomic DNA, such as an entire genome.

In additional embodiments of the present invention, there is a method of amplifying a genome, comprising the steps of obtaining at least one DNA molecule; randomly fragmenting the DNA molecule to produce DNA fragments; modifying the ends of the DNA fragments to provide attachable ends; attaching an adaptor having a known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor; extending the 3′ end of the modified DNA from the nick site; and amplifying a plurality of the adaptor-linked fragments.

In further embodiments, there is a method of generating a library, comprising the steps of obtaining at least one DNA molecule; randomly fragmenting the DNA molecule to produce DNA fragments; modifying the ends of the DNA fragments to provide attachable ends; attaching an adaptor having a known sequence and a nonblocked 3′ end to both ends of a plurality of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor; extending the 3′ end of the modified DNA from the nick site. The method may further comprise the step of amplifying a plurality of the adaptor-linked fragments.

Other embodiments of the present invention include a method of preparing at least one DNA molecule, comprising admixing together: an endonuclease; a ligase; an adaptor; and a buffer, under conditions wherein the DNA molecule, such as a genome, is cleaved by the endonuclease to generate a plurality of DNA fragments, a plurality of the ends of which are ligated to the adaptor. The method may consist essentially of one step. The cleavage and ligation may occur substantially concomitantly. In a particular embodiment, the ligation occurs under the same reaction conditions as the cleavage. In another particular embodiment, the ligation step occurs without changing the buffer following the cleavage step and/or the method lacks DNA precipitation. The endonuclease may be deoxyribonuclease I or a Cvi restriction endonuclease, and the ligase may be T4 DNA ligase.

In a specific embodiment, the adaptor is a blunt end adaptor, a 5′ overhang adaptor, a 3′ overhang adaptor, or a mixture thereof. The adaptor may comprise a first primer and a second primer, said first primer greater in length than said second primer. The first primer may lack a 5′ phosphate, the second primer may lack a 5′ phosphate group, or both first and second primers lack 5′ phosphate groups. The buffer comprises a divalent cation, a salt, adenosine triphosphate, dithiothreitol, or a mixture thereof, in a specific embodiment.

In a particular embodiment, the conditions comprise a large molar excess of linkers to DNA fragment ends, such as at least about 10-fold to about 100-fold. The method may further comprise amplifying the DNA fragments using a primer complementary to the adaptor.

In another embodiment of the present invention, there is a method of generating a library of DNA molecules comprising admixing together: at least one DNA molecule; an endonuclease; a ligase; an adaptor; and a buffer, under conditions wherein said DNA molecule is cleaved by said endonuclease to generate a plurality of DNA fragments, a plurality of the ends of which are ligated to said adaptor.

In an additional embodiment of the present invention, there is a kit for performing a concomitant endonuclease/ligase reaction, comprising an endonuclease; a ligase; an adaptor, as described elsewhere herein; and a buffer.

In another embodiment, there is a method of diagnosing a condition in an individual, comprising the step of obtaining at least one DNA molecule from said individual; randomly fragmenting the DNA molecule to produce DNA fragments; modifying the ends of the DNA fragments to provide attachable ends; attaching an adaptor having a known sequence and a nonblocked 3′ end to the ends of the modified DNA fragments to produce adaptor-linked fragments, wherein the 5′ end of the DNA is attached to the nonblocked 3′ end of the adaptor, leaving a nick site between the juxtaposed 3′ end of the DNA and a 5′ end of the adaptor; extending the 3′ end of the modified DNA from the nick site; amplifying at least one adaptor-linked fragment; and identifying a DNA sequence in said fragment that is representative of said condition. The DNA sequence in the fragment may comprise at least a portion of an X chromosome or a Y chromosome, and the DNA sequence may be a point mutation, a deletion, an inversion, a repeat, or a combination thereof.

In another embodiment of the present invention, there is a method of amplifying at least one RNA molecule, comprising the steps of obtaining at least one RNA molecule; reverse transcribing the RNA molecule to produce a cDNA molecule; randomly fragmenting the cDNA molecule to produce DNA fragments; modifying the ends of the DNA fragments to provide attachable ends; attaching an adaptor having a known sequence and a nonblocked 3′ end to the ends of the modified DNA fragments to produce adaptor-linked fragments, wherein the 5′ end of the DNA is attached to the nonblocked 3′ end of the adaptor, leaving a nick site at the juxtaposed 3′ end of the DNA and a 5′ end of the adaptor; extending the 3′ end of the modified DNA from the nick site; and amplifying a plurality of the adaptor-linked fragments.

In an additional embodiment, there is a method of amplifying a population of DNA molecules comprised in a plurality of populations of DNA molecules, the method comprising the steps of obtaining a plurality of populations of DNA molecules, wherein at least one population in said plurality comprises DNA molecules having in a 5′ to 3′ orientation the following: a known identification sequence specific for said population; and a known primer amplification sequence; and amplifying said population of DNA molecules by polymerase chain reaction, said reaction utilizing a primer for said identification sequence. The obtaining step may be further defined as obtaining a population of DNA molecules, said molecules comprising a known primer amplification sequence; amplifying said DNA molecules with a primer having in a 5′ to 3′ orientation the following: the known identification sequence; and the known primer amplification sequence; and mixing said population with at least one other population of DNA molecules. The population of DNA molecules is a genome, in specific embodiments.

In an additional embodiment of the present invention, there is a method of amplifying a population of DNA molecules comprised in a plurality of populations of DNA molecules, the method comprising the steps of obtaining a plurality of populations of DNA molecules, wherein at least one population in the plurality comprises DNA molecules, wherein the 5′ ends of said DNA molecules comprise in a 5′ to 3′ orientation the following: a single-stranded region comprising a known identification sequence specific for the population; and a known primer amplification sequence; and isolating the population through binding of at least part of the single stranded known identification sequence of a plurality of the DNA molecules to a surface; and amplifying the isolated DNA molecules by polymerase chain reaction, said reaction utilizing a primer for the primer amplification sequence.

The obtaining step may be further defined as obtaining a population of DNA molecules, said molecules comprising a known primer amplification sequence; amplifying said DNA molecules with a primer comprising in a 5′ to 3′ orientation the following: the known identification sequence; a non-replicable linker; and the known primer amplification sequence; and mixing said population with at least one other population of DNA molecules. The isolating step may be further defined as binding at least part of the single stranded known identification sequence to an immobilized oligonucleotide comprising a region complementary to the known identification sequence.

In an additional embodiment of the present invention, there is a method of immobilizing an amplified genome, comprising the steps of obtaining an amplified genome, wherein a plurality of DNA molecules from the genome comprise a known primer amplification sequence at both the 5′ and 3′ ends of the molecules; and attaching a plurality of the DNA molecules to a support. The attaching step may be further defined as comprising covalently attaching the plurality of DNA molecules to the support through the known primer amplification sequence. The covalently attaching step may be further defined as hybridizing a region of at least one single stranded DNA molecules to a complementary region in the 3′ end of a oligonucleotide immobilized to the support; and extending the 3′ end of the oligonucleotide to produce a single stranded DNA/extended polynucleotide hybrid. The method may further comprise the step of removing the single stranded DNA molecule from the single stranded DNA/extended polynucleotide hybrid to produce an extended polynucleotide.

In specific embodiments, the method further comprises the step of replicating the extended polynucleotide. The replicating step may be further defined as providing to the extended polynucleotide a DNA polymerase and a primer complementary to the known primer amplification sequence; extending the 3′ end of the primer to form an extended primer molecule; and releasing said extended primer molecule.

In an additional embodiment of the present invention, there is a method of immobilizing an amplified genome, comprising the steps of obtaining an amplified genome, wherein a plurality of DNA molecules from the genome comprise a tag; and a known primer amplification sequence at both the 5′ and 3′ ends of the molecules; and attaching a plurality of the DNA molecules to a support. In a specific embodiment, the attaching step is further defined as comprising attaching the plurality of DNA molecules to the support through the tag, which in some embodiments is biotin and the support comprises streptavidin. The tag may comprise an amino group or a carboxyl group. The tag may comprise a single stranded region and the support may comprise an oligonucleotide comprising a sequence complementary to a region of the tag.

In specific embodiments, the single stranded region is further defined as comprising an identification sequence. The DNA molecules may be further defined as comprising a non-replicable linker that is 3′ to the identification sequence and that is 5′ to the known primer amplification sequence. The method may also further comprise the step of removing contaminants from the immobilized genome.

In a specific embodiment of the present invention, a method may comprise the incorporation of a tag, such as a functional tag. For example, the functional tag may serve to suppress library amplification with a terminal priming sequence. The terminal sequence may be introduced by ligation of adaptor sequence. In another embodiment, the terminal sequence may be introduced by enzymatic tailing, for example with terminal transferase. In a preferred embodiment, the terminal sequence may be introduced during PCR amplification with a primer comprised of a universal proximal sequence and a specific non-complementary tail. Non-complementary tails may, for example, be comprised of a region of poly cytosine where the C-tail may be from about 1-30 bases in length. As described in U.S. Patent Application Publication 20030143599, herein incorporated by reference in their entirety, genomic DNA libraries flanked by homopolymeric tails consisting of G/C base paired double stranded DNA are suppressed in amplification with single polyC primer. This suppression effect is moderated when balanced with a second site-specific primer, whereby amplification of a plurality of fragments containing the unique priming site and the universal terminal sequence are amplified selectively using a specific primer and a poly-C primer, for instance C₁₀. Those skilled in the art will recognize that genomic complexity may dictate the requirement for sequential or nested amplifications to amplify a single species of DNA from the library to purity.

In a particular aspect of the invention, there is a method of preparing a DNA molecule, comprising obtaining a population of DNA molecules having ligatable ends of unknown nature; providing to the population one or more known forms of adaptors, wherein the adaptors each comprise at least one known sequence and at least one oligonucleotide having a 3′ extendable end; determining ligatability of the one or more known forms of adaptors to the DNA molecules; and ligating the known one or more forms of adaptors to the DNA molecule. The determining step may be further defined as identifying a ratio of ligatable forms of adaptors corresponding to the nature of the ends of the DNA molecules in the population, and wherein the ligating step is further defined as introducing to the population a plurality of the adaptors in said ratio. The ligatability of the one or more forms of adaptors may be determined separately or concomitantly. The population of DNA molecules may derive from plasma, serum, or a combination thereof.

The method may further comprise the step of extending the 3′ end of the oligonucleotide by polymerization to produce an extended product, which may be amplified by polymerase chain reaction. The population of DNA molecules may be obtained from serum or from plasma, in particular embodiments.

In other embodiments, the present invention encompasses a DNA molecule or a plurality of DNA molecules (which may be referred to as a library) generated by methods described herein.

In an additional aspect of the invention, there is a method of sequencing genomic DNA from a limited source of material by obtaining at least one DNA molecule from a limited source of material; randomly fragmenting the DNA molecule to produce DNA fragments; modifying the ends of the DNA fragments to provide attachable ends; attaching an adaptor having a known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor; extending the 3′ end of the modified DNA from the nick site; amplifying a plurality of the adaptor-linked fragments; providing from the plurality of the adaptor-linked fragments a first sample of adaptor-linked fragments and a second sample of adaptor-linked fragments; sequencing at least some of the adaptor-linked fragments from the first sample; incorporating homopolymeric sequence to the ends of the adaptor-linked fragments from the second sample; amplifying at least some of the adaptor-linked fragments from the second sample utilizing a first primer complementary to the homopolymeric sequence and a second primer complementary to a specific sequence in the adaptor-linked fragments from the second sample; and analyzing at least some of the amplified sequence.

In particular embodiments, the incorporating of the homopolymeric sequence comprises one of the following steps extending the 3′ end of the adaptor-linked fragments by terminal deoxynucleotidyl transferase; ligating an adaptor comprising the homopolymeric sequence to the ends of the adaptor-linked fragments; or replicating the adaptor-linked fragments with a primer comprising the homopolymeric sequence at its 5′ end. In other particular embodiments, the sequencing step is further defined as cloning the adaptor-linked fragments from the first sample into a vector; and sequencing at least some of the cloned adaptor-linked fragments from the first sample. The specific sequence of the DNA molecule may be provided by the sequencing step of the adaptor-linked fragments from the first sample.

In some embodiments of the present invention, there is a limited source of material from which to process using the methods and compositions described herein. For example, the limited source of material may be a microorganism substantially resistant to culturing, an extinct species, a single DNA molecule, a single cell, a single chromosome, and so forth.

In specific embodiments of the present invention, compositions are added during the library and/or amplification step(s) to facilitate completion of the appropriate steps. For example, compositions, which may be referred to as additives, are included in some reactions to melt DNA strands that are substantially resistant to melting, such as GC-rich regions. In particular embodiments, these additives facilitate polymerization through GC-rich DNA. A skilled artisan recognizes that there are agents that decrease melting temperature, such as to prevent, reduce, or facilitate overcoming the formation of secondary structure. Examples of such an agent include dimethyl sulfoxide or betaine. Another type of agent is a nucleotide analog that when present in a strand does not form or contribute to secondary structure as readily as a dGTP, such as 7-Deaza-dGTP.

Other objects, features and advantages of the present invention will become apparent from the following detailed description. It should be understood, however, that the detailed description and the specific examples, while indicating the preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 demonstrates preparation of a library by mechanical fragmentation. Briefly, genomic DNA is fragmented mechanically resulting in the production of double stranded DNA fragments with blocked 3′ ends (represented as X). The ends are repaired (also referred to as “polished”) resulting in the generation of, for example, blunt or 1 bp overhangs at both ends. Adaptor sequences are ligated to the 5′ ends of each side of the DNA fragment. Finally, an extension step is performed to displace the short, 3′ blocked adaptor and extend the DNA fragment across the ligated adaptor sequence.

FIG. 2 illustrates preparation of a library by chemical fragmentation using a non-strand displacing polymerase. Briefly, genomic DNA is fragmented chemically resulting in the production of single stranded DNA fragments with blocked 3′ ends (represented as X). A fill-in reaction with a non-strand displacing polymerase is performed. The resulting ds DNA fragments have blunt or one to several bp overhangs at each end and may contain nicks of the newly synthesized DNA strand at the points where the 3′ end of an extension product meets the 5′ end of a distal extension product. Adaptor sequences are ligated to the 5′ ends of each side of the DNA fragment. Finally, an extension step is performed to displace the short, 3′ blocked adaptor and extend the DNA fragment across the ligated adaptor sequence. This process will result in only one competent strand for amplification if there are nicks present in the strand created during the fill-in reaction.

FIG. 3 represents an alternative model by which a library is prepared by chemical fragmentation using a strand-displacing polymerase. Briefly, genomic DNA is fragmented chemically resulting in the production of single stranded DNA fragments with blocked 3′ ends (represented as X). A fill-in reaction with a strand displacing polymerase is performed. The resulting DNA fragments will have a branched structure resulting in the creation of additional ends. Most (if not all) ends will comprise either blunt or several bp overhangs. Adaptor sequences are ligated to the 5′ ends of each end of the DNA fragments. Finally, an extension step is performed to displace the short, 3′ blocked adaptor and extend the DNA fragment across the ligated adaptor sequence. This process may result in multiple strands of different sizes being competent to undergo subsequent amplification, depending on the amount of strand displacement that occurs. In the example depicted, the full-length parent strand and the most 3′ distal daughter strand will be competent to undergo amplification.

FIG. 4 represents an alternative model by which a library is prepared by chemical fragmentation using a polymerase with nick translation ability. Briefly, genomic DNA is fragmented chemically resulting in the production of single stranded DNA fragments with blocked 3′ ends (represented as X). A fill-in reaction with a polymerase capable of nick translation is performed. The resulting ds DNA fragments have blunt or several bp overhangs at each end and the daughter strand will be one continuous fragment. Adaptor sequences are ligated to the 5′ ends of each side of the DNA fragment. Finally, an extension step is performed to displace the short, 3′ blocked adaptor and extend the DNA fragment across the ligated adaptor sequence. Both strands of the DNA fragment will be suitable for amplification due to the creation of a full-length daughter strand by nick translation during the fill-in reaction.

FIGS. 5A and 5B illustrate the structure of various exemplary adaptor sequences used in library preparation. In FIG. 5A, there are structures of the blunt-end, 5′ overhang, and 3′ overhang adaptors. In FIG. 5B, there is sequence of the T7HEG oligo and structure of the exemplary T7HEG adaptor following annealing.

FIG. 6 shows the structure of a specific exemplary adaptor and how it is ligated to blunt-ended double stranded DNA fragments, the resulting ds DNA fragments, and the extension step following ligation used to fill in the adaptor sequence and displace the blocked short adaptor.

FIGS. 7A and 7B show the amplification curves of libraries generated from mechanically fragmented DNA (FIG. 7A) and gel analysis of the resulting products following purification (FIG. 7B). In FIG. 7A, amplification curves were generated using the I-Cycler real-time detection system in conjunction with SYBR Green I. Curves are graphed as % max relative fluorescence units (% Max RFU) and maximal DNA production has been determined by spectrophotometric measurement to occur at the point where the % Max RFU decreases. In FIG. 7B, there is a 1.5% TBE agarose gel electrophoresis of 200 ng of amplified products indicating a size distribution of 500 bp to 3 kb similar to the mechanically fragmented starting material.

FIGS. 8A and 8B demonstrate typical distributions of specific DNA sites in primary (FIG. 8A) and secondary (FIG. 8B) amplified libraries. Histograms are generated based on the fold of amplification for each of 103 human genomic STS markers quantified by Real-Time PCR.

FIGS. 9A and 9B represent the amplification curves of libraries generated from DNA fragmented chemically (FIG. 9A) and gel analysis of amplified products from chemically fragmented libraries using either universal adaptors (u) or T7HEG (h) adaptors (FIG. 9B). In FIG. 9A, amplification curves were generated using the I-Cycler real-time detection system in conjunction with SYBR Green I. Curves are graphed as % max relative fluorescence units (% Max RFU) and maximal DNA production has been determined by spectrophotometric measurement to occur at the point where the % Max RFU decreases. In FIG. 9B, 1.5% TBE agarose gel electrophoresis of 200 ng of amplified products indicates a size distribution of 100 bp to greater than 3 kb.

FIG. 10 provides a method of converting duplex DNA into end-linkered, amplifiable fragments. Duplex DNA, linkers, double-stranded DNA endonuclease, and ligase are incubated in an optimized buffer system compatible with both enzymes. Endonuclease cleavage will produce DNA fragment ends with 5′-phosphate and 3′-hydroxyl termini. Linkers are ligated to these ends, such that only one strand of the duplex linker is covalently attached to each fragment end. Since the kinetics of ligation are as rapid as cleavage, successive rounds of cleavage and ligation will eventually lead to a randomly fragmented, end-linkered DNA library of desired size distribution.

FIGS. 11A through 11C illustrate exemplary linker designs. Linkers are preferably designed with non-phosphorylated 5′-termini so that linker-linker ligation cannot occur. In specific embodiments, one of the oligonucleotides is shorter than the other. In FIG. 11A, linker designed to ligate to blunt-ended DNA fragments is utilized. In FIG. 11B, linker designed to ligate to DNA fragments with 5′ overhangs is utilized. In FIG. 11C, linker designed to ligate to DNA fragments with 3′ overhangs is utilized. The N represents either specific bases, for use with sequence-specific endonucleases, or any of all four bases, for use with sequence-independent endonucleases. Typically, there is about one or two N bases on the overhang linkers.

FIGS. 12A through 12B show endonuclease cleavage by DNase I in Buffer M10 and M3. FIG. 12A shows a 1.0% TBE agarose gel of 200 ng human genomic DNA digested by DNase I in Buffer M10. DNA was digested for 15′ (Lanes 1-3) or 1 hour (Lanes 4-6) in 20 μL of Buffer M10 at 16° C. The DNA was treated with 5×10⁻⁵ U/μL (Lanes 1, 4), 3.75×10⁻⁴ U/μL (Lanes 2, 5), or 2.5×10⁻⁵ U/μL (Lanes 3, 6) DNase I. FIG. 12B shows a 1.0% TBE agarose gel of 80 ng human genomic DNA digested by DNase I in Buffer M3. 200 ng DNA was digested in 20 μL for 3 hours at 16° C. with 3×10⁻⁵ U/μL DNase I.

FIGS. 13A through 13E show exemplary linkers used in conjunction with DNase I endonuclease. In FIG. 13A, a linker designed to ligate to blunt-ended DNA fragments is utilized. In FIGS. 13B and 13C, linkers designed to ligate to DNA fragments with single- or two-base 5′ overhangs are utilized. In FIGS. 13D and 13E, linkers designed to ligate to DNA fragments with single- or two-base 3′ overhangs are utilized. N represents the four bases, A, G, C, and T. X represents a 3′-amino group.

FIG. 14 shows average fragment size of libraries constructed in Buffer M3. A 1.0% TBE agarose gel was electrophoresed with 80 ng of human genomic DNA converted into a library in Buffer M3. One hundred ng of DNA was digested in 10 μL for 18 hours at 16° C. with 1×10⁻⁵ U/μL DNase I (Lane 1), 2×10⁻⁵ U/μL DNase I (Lane 2), or 3×10⁻⁵ U/μL DNase I (Lane 3), in the presence of 1,000 Units of T4 DNA Ligase and 10 picomoles of each linker described in FIG. 13.

FIGS. 15A-15C describes amplification of end-linkered DNA fragments. FIG. 15A shows real-time PCR amplification kinetics of genomic DNA converted into a library in Buffer M3 or Buffer M10. FIG. 15B shows a 1.0% TBE agarose gel of amplified product from libraries constructed in Buffer M3. Lanes 1-3 correspond to products amplified from libraries described in FIG. 14, Lanes 1-3. FIG. 15C shows a 1.0% TBE agarose gel of amplified product from libraries constructed at different time points in Buffer M10. The libraries were constructed by incubation for 1 hour in Buffer M10 (Lane 1), 6 hours in Buffer M10 (Lane 2), or 21 hours in Buffer M10 (Lane 3).

FIGS. 16A through 16C show the structure of the universal primer with identification (ID) tags. FIG. 16A illustrates replicable universal primer with the universal primer sequence U at the 3′ end and individual ID sequence tag T at the 5′ end. FIG. 16B shows non-replicable universal primer with the universal primer sequence U at the 3′ end, individual ID sequence tag T at the 5′ end, and non-replicable organic linker L between them. FIG. 16C shows 5′ overhanging structure of the ends of DNA fragments in the WGA library after amplification with a non-replicable universal primer.

FIG. 17 shows the process of synthesis of WGA libraries with the replicable ID tag and their usage, such as for security and/or confidentiality purposes, by mixing several libraries and recovering an individual library by ID-specific PCR.

FIG. 18 shows the process of synthesis of WGA libraries with the non-replicable ID tag and their usage, such as for security and/or confidentiality purposes, by mixing several libraries and recovering an individual library by ID-specific hybridization capture.

FIG. 19 shows the process for covalent immobilization of WGA library on a solid support.

FIGS. 20A and 20B show WGA libraries in the micro-array format. FIG. 20A illustrates an embodiment utilizing covalent attachment of the libraries to a support. FIG. 20B illustrates an embodiment utilizing non-covalent attachment of the libraries to a support.

FIG. 21 shows an embodiment wherein the immobilized WGA library is used repeatedly.

FIG. 22 describes the method of WGA product purification utilizing a non-replicable universal primer and magnetic beads affinity capture.

FIG. 23A demonstrates preparation of a library from serum or plasma DNA. Briefly, genomic DNA isolated from either serum or plasma is treated with a polymerase containing both 5′ polymerase and 3′ exonuclease activities in order to generate blunt ends. Adaptor sequences are ligated to the 5′ ends of each side of the DNA fragment. Finally, an extension step is performed to displace the short, 3′ blocked adaptor and extend the DNA fragment across the ligated adaptor sequence and the resulting molecules are amplified by PCR. FIG. 23B reveals the primer sequence (Yb8 Forward: 5′-CGAGGCGGGTGGATCATGAGGT-3′, SEQ ID:48; Yb8 Reverse: 5′-TCTGTCGCCCAGGCCGGACT-3′, SEQ ID:49) used to quantify DNA isolated from serum or plasma. These primers amplify a single DNA product that correlates to the Yb8 subfamily of alu genes that is represented approximately 1,852 times in the genome (Walker et al., 2003).

FIGS. 24A and 24B display the amplification curves of libraries generated from DNA isolated from serum (FIG. 24A) and plasma (FIG. 24B). The amplification curves were generated using the I-Cycler real-time detection system in conjunction with SYBR Green I. Curves are graphed as % max relative fluorescence units (% Max RFU). It should be noted that the I-Cycler software does not provide data for the last cycle run. Thus, the number of cycles of PCR performed is one more than indicated on the graph.

FIGS. 25A and 25B represent gel analysis of serum (FIG. 25A) and plasma (FIG. 25B) DNA and the amplified products following WGA from serum and plasma DNA. In FIG. 25A, the results of 1% TBE agarose gels of serum DNA (5 ng) and amplified serum DNA (200 ng) indicate a size range of 200 bp to 2 kb for the serum DNA and 200 bp to 1 kb for the amplified DNA. In FIG. 25B, gel analysis of plasma DNA on a 1% TBE gel indicates that the products are contained in two size fractions. One fraction is 200 bp to 1 kb, while the second is greater than 10 kb. Analysis of the amplified plasma DNA indicates a size range of 200 bp to 1 kb, suggesting that this is the only fraction in the starting plasma DNA that is able to be amplified.

FIG. 26 demonstrates real-time STS analysis of serum DNA and amplified products from serum and plasma DNA. The normalized values are calculated by dividing the measured value by the average value for that sample. The solid line across the entire graph represents the average, while the short line in each column represents the median value. For serum DNA, all 8 sites tested were within a factor of 2 of the mean, while for the amplified DNA samples all 8 sites were within a factor of 4 of the mean. It should be noted that the relative pattern of representation of specific STS sites was maintained between the serum DNA and the amplified products. For amplified plasma DNA, all 16 sites were within a factor of 5 of the mean amplification. Analysis of plasma DNA was not performed due to the low recovery of DNA from plasma samples.

FIG. 27 demonstrates preparation of a library from serum or plasma DNA. Briefly, adaptor sequences are ligated to the 5′ ends of each side of DNA fragments isolated from serum or plasma. The adaptor sequences contain a specific mix of 5′ N and 3, N overhangs that allow optimal annealing and ligation of the adaptor complex to the template DNA. Finally, an extension step is performed to displace the short, 3′ blocked adaptor and extend the DNA fragment across the ligated adaptor sequence and the resulting molecules are amplified by PCR. In this method, Pfu can also be added during the extension step to remove any 3′ bases present on the template molecule that are not complementary to the adaptor sequence. This addition results in improved efficiency of the PCR amplification, indicating that more molecules are successfully filled in during the extension step. Finally, molecules containing adaptors at both ends are amplified using PCR.

FIG. 28 illustrates the adaptor sequences utilized during ligation. Optimal ligation can be obtained using the 5′ T7N adaptors N2T7 and N5 T7 combined with the 3′ T7N adaptors T7N2 and T7N5. However, it should be observed that acceptable results are obtained with a variety of combinations of adaptors as long as at least one adaptor containing a 5′ N overhang and one adaptor containing a 3′ N overhang are utilized together.

FIGS. 29A and 29B display the amplification curves of libraries generated from DNA isolated from serum (FIG. 29A) and plasma (FIG. 29B). The amplification curves were generated using the I-Cycler real-time detection system in conjunction with SYBR Green I. Curves are graphed as % max relative fluorescence units (% Max RFU). It should be noted that the I-Cycler software does not provide data for the last cycle run. Thus, the number of cycles of PCR performed is one more than indicated on the graph.

FIG. 30 represents gel analysis of amplified products created from serum and plasma DNA. The results of 1% TBE agarose gels of serum and plasma WGA products (5 ng) indicate a size range of 200 bp to 2 kb for both the serum and plasma DNA. These results are similar to the size range obtained using ligation of blunt end adaptors following polishing of serum and plasma DNA illustrated in FIG. 25.

FIG. 31 demonstrates real-time STS analysis of serum DNA and amplified products from serum and plasma DNA. The normalized values are calculated by dividing the measured value by the average value for that sample. The solid line across the entire graph represents the average, while the short line in each column represents the median value. For amplified serum DNA, all 16 sites tested were within a factor of 7 of the mean, and 15 of 16 sites were within a factor of 4. For amplified plasma DNA, all 16 sites were within a factor of 6 of the mean amplification. Notice that there is a similar range of distribution of STS sites in amplified material from 5 ng of serum DNA and 1 ng of plasma DNA.

FIG. 32 shows microarray hybridization analysis of the single-cell DNA produced by whole genome amplification.

FIG. 33 illustrates single-cell DNA arrays: detection and analysis of cancer cells.

FIG. 34 displays the amplification curves of libraries generated from genomic DNA where libraries were prepared in the presence (▪,□) or absence (●,∘) of 4% DMSO/0.2 mM N⁷-dGTP and amplified in the presence (▪,●) or absence (□,∘) of 4% DMSO/0.2 mM N⁷-dGTP. The addition of DMSO and N7-dGTP during library amplification resulted in a one cycle shift to the right.

FIG. 35 demonstrates real-time STS analysis of normal and GC-rich STS sites in amplified products from genomic DNA. The solid line crossing the entire graph represents the amount of DNA added to the STS assay based on optical density. The thick line in each column represents the average value while the thin line represents the median value obtained by real-time PCR STS analysis. For DNA amplified in the absence of DMSO and N⁷-dGTP, 8 of the 11 GC-rich markers were underrepresented. Addition of DMSO and N⁷-dGTP during library preparation increased the values of the majority of GC-rich STS, although not to the level of the normal STS sites. However, addition of DMSO and N⁷-dGTP only during library amplification resulted in the majority of GC-rich STS sites being amplified to similar levels as the normal STS sites, with a couple of exceptions. Finally, addition of DMSO and N⁷-dGTP during both library preparation and amplification resulted in all sites being represented within a factor of 4 of the mean amplification and represented the tightest distribution of all STS sites of any methods utilized.

FIGS. 36A through 36C show the process of conversion of amplified WGA libraries into libraries with additional Gₙ or C₁₀ sequence tag located at the 3′ or 5′ end of the universal known primer sequence U, respectively, with subsequent use of these modified WGA libraries for targeted amplification of one or several specific genomic sites using universal primer C₁₀ and unique primer P. FIG. 36A shows library tagging by incorporation of a (dG)n tail using TdT enzyme; FIG. 36B demonstrates library tagging by ligation of an adaptor with the C₁₀ sequence at the 5′ end of the long oligonucleotide; FIG. 36C shows library tagging by secondary replication of the WGA library using known primer U with the C₁₀ sequence at the 5′ end.

FIGS. 37A and 37B show the inhibitory effect of poly-C tags on amplification of synthesized WGA libraries. FIG. 37A shows real-time PCR amplification chromatograms of different length poly-C tags incorporated by polymerization. FIG. 37B shows delayed kinetics or suppression of amplification of C-tagged libraries amplified with corresponding poly-C primers.

FIGS. 38A and 38B display real-time PCR results of targeted amplification using a specific primer and the universal C₁₀ tag primer. FIG. 38A shows the sequential shift with primary and secondary specific primers with a combined enrichment above input template concentrations. FIG. 38B shows the effect of specific primer concentration on selective amplification. Real-time PCR curves show a gradient of specific enrichment with respect to primer concentration.

FIGS. 39A and 39B detail the individual specific site enrichment for each unique primary oligonucleotide in the multiplexed targeted amplification. FIG. 39A shows values of enrichment for each site relative to an equal amount of starting template, while FIG. 39B displays the same data as a histogram of frequency of amplification.

FIG. 40A shows the analysis of secondary “nested” real-time PCR results for 45 multiplexed specific primers. Enrichment is expressed as fold amplification above starting template ranging from 100,000 fold to over 1,000,000 fold. FIG. 40B shows the distribution frequency for all 45 multiplexed sites.

FIGS. 41A through 41G illustrate the schematic representation of a whole genome sequencing application using tagged libraries synthesized from limited starting material. Libraries provide a means to recover precious or rare samples in an amplifiable form that can function both as substrate for cloning approaches and through conversion to C-tagged format a directed sequencing template for gap filling and primer walking.

FIG. 42 depicts a schematic representation of creation and amplification of a secondary genome library containing a specific subset of genomic regions contained within the primary whole genome library. Genomic DNA is converted into a primary library containing a universal priming site U. Homopolymeric Poly-C tails (C) are added to either the library or the amplified products by means described in FIG. 36 and Example 16. The products of amplification containing the homopolymeric poly-C tails are digested with a nuclease targeted at specific sequences, such as a restriction site or a methylation site. Following digestion, a second universal adaptor (V) is attached to the ends resulting from digestion. Amplification of the secondary genomic library is accomplished by PCR using primers C and U. Amplification of molecules containing the sequence for primer C at both ends is inhibited.

### DETAILED DESCRIPTION OF THE INVENTION

In keeping with long-standing patent law convention, the words “a” and “an” when used in the present specification in concert with the word comprising, including the claims, denote “one or more.”

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

U.S. Provisional Patent Application No. 60/453,060, filed Mar. 7, 2003 is hereby incorporated by reference herein in its entirety. U.S. Nonprovisional Patent Application No. Unknown but claiming priority to U.S. Provisional Patent Application No. 60/453,060, filed concurrently herewith, and entitled, “AMPLIFICATION AND ANALYSIS OF WHOLE GENOME AND WHOLE TRANSCRIPTOME LIBRARIES GENERATED BY DNA POLYMERIZATION PROCESS” is also hereby incorporated by reference herein in its entirety.

I. Definitions

The term “attachable ends” as used herein refers to DNA ends (that are preferably blunt ends or comprise short overhangs on the order of about 1 to about 3 nucleotides) in which an adaptor is able to be attached thereto. A skilled artisan recognizes that the term “attachable ends” comprises ends that are ligatable, such as with ligase, or that are able to have an adaptor attached by non-ligase means, such as by chemical attachment.

The term “base analog” as used herein refers to a compound similar to one of the four DNA nitrogenous bases (adenine, cytosine, guanine, thymine, and uracil) but having a different composition and, as a result, different pairing properties. For example, 5-bromouracil is an analog of thymine but sometimes pairs with guanine, and 2-aminopurine is an analog of adenine but sometimes pairs with cytosine. Another analog, nitroindole, is used as a “universal” base” that pairs with all other bases.

The term “backbone analog” as used herein refers to a compound wherein the deoxyribose phosphate backbone of DNA has been modified. The modifications can be made in a number of ways to change nuclease stability or cell membrane permeability of the modified DNA. For example, peptide nucleic acid (PNA) is a new DNA derivative with an amide backbone instead of a deoxyribose phosphate backbone. Other examples in the art include methylphosphonates.

The term “blocked 3′ end” as used herein is defined as a 3′ end of DNA lacking a hydroxyl group.

The term “blunt end” as used herein refers to an end of a ds DNA molecule having 5′ and 3′ ends, wherein the 5′ and 3, ends terminate at the same nucleotide position. Thus, the blunt end comprises no 5′ or 3′ overhang. A ds DNA molecule may comprise a blunt end on one or both ends.

The term “DNA immortalization” as used herein is defined as the conversion of a mixture of DNA molecules into a form that allows repetitive, unlimited amplification without loss of representation and/or without size reduction. In a specific embodiment, the mixture of DNA molecules is comprised of multiple DNA sequences.

The term “fill-in reaction” as used herein refers to a DNA synthesis reaction that is initiated at a 3′ hydroxyl DNA end and leads to a filling in of the complementary strand. The synthesis reaction comprises at least one polymerase and dNTPs (dATP, dGTP, dCTP and dTTP). In a specific embodiment, the reaction comprises a thermostable DNA polymerase.

The term “genome” as used herein is defined as the collective gene set carried by an individual, cell, or organelle.

The term “nonreplicable organic chain” as used herein is defined as any link between bases that can not be used as a template for polymerization, and, in specific embodiments, arrests a polymerization/extension process.

The term “non strand-displacing polymerase” as used herein is defined as a polymerase that extends until it is stopped by the presence of, for example, a downstream primer. In a specific embodiment, the polymerase lacks 5′-3′ exonuclease activity.

The term “random fragmentation” as used herein refers to the fragmentation of a DNA molecule in a non-ordered fashion, such as irrespective of the sequence identity or position of the nucleotide comprising and/or surrounding the break.

The term “random primers” as used herein refers to short oligonucleotides used to prime polymerization comprised of nucleotides, at least the majority of which can be any nucleotide, such as A, C, G, or T.

The term “strand-displacing polymerase” as used herein is defined as a polymerase that will displace downstream fragments as it extends. In a specific embodiment, the polymerase comprises 5′-3′ exonuclease activity.

The term “thermophilic DNA polymerase”, as used herein refers to a heat-stable DNA polymerase.

II. The Present Invention

A. Whole Genome Amplification using Fragmented Genomic DNA and Adaptors

In this embodiment, there are methods of preparing a library of DNA molecules in such a way as to enable the non-biased amplification of all molecules within the library by PCR utilizing a primer comprising a known sequence. The method of fragmentation of the parent DNA defines the manner in which the library is created. Two distinct methods of library preparation are presented based on three methods of DNA fragmentation. Other methods of fragmentation, well-known in the art, which would result in fragments with similar properties (i.e. single stranded vs. double stranded), would also allow the production of libraries using the appropriate methods detailed here.

In a specific embodiment, the DNA is randomly fragmented in such a way as to result in the production of double stranded DNA fragments. A skilled artisan recognizes that such fragmentation would result in a smear on a gel. The present invention is designed to attach adaptors comprising known sequence (such as for subsequent amplification) to a plurality of DNA fragments regardless of size and amplify these DNA fragments without bias.

In another embodiment, the DNA is randomly fragmented in such a way as to result in the production of single stranded DNA fragments. A skilled artisan recognizes that such fragmentation would result in a smear on a gel. The present invention is designed to convert the single stranded fragments into DNA fragments that are double stranded at both ends. This conversion to double stranded ends allows the efficient attachment of adaptors to a plurality of DNA fragments regardless of size. This method may also result in the production of additional DNA fragments that are smaller than the original DNA fragments and that are also competent to have adaptors attached to them. Due to the random nature of these DNA fragments, these additional DNA fragments will represent all regions of original DNA and will not introduce bias into the amplification.

1. Preparation of Randomly Fragmented DNA

Generally, a library is prepared in at least 4 steps: first, randomly fragmenting the DNA into pieces, such as with an average size between about 500 bp and about 4 kb; second, repairing the 3′ ends of the fragmented pieces and generating blunt, double stranded ends; third, attaching universal adaptor sequences to the 5′ ends of the fragmented pieces; and fourth, filling in of the resulting 5′ adaptor extensions. In an alternative embodiment, the first step comprises obtaining DNA molecules defined as fragments of larger molecules, such as may be obtained from a tissue (blood, urine, feces, and so forth), a fixed sample, and the like, and may comprise degraded DNA. Such DNA may comprise lesions including double or single stranded breaks.

A skilled artisan recognizes that random fragmentation can be achieved by at least three exemplary means: mechanical fragmentation, chemical fragmentation, and/or enzymatic fragmentation.

2. Repairing of the 3′ Ends of the Fragmented Pieces and Generation of Blunt Double Stranded Ends

a. Repair of Mechanically Fragmented DNA

Mechanical fragmentation can occur by any method known in the art, including hydrodynamic shearing of DNA by passing it through a narrow capillary or orifice (Oefner et al., 1996; Thorstenson et al., 1998), sonicating the DNA, such as by ultrasound (Bankier, 1993), and/or nebulizing the DNA (Bodenteich et al., 1994). Mechanical fragmentation usually results in double strand breaks within the DNA molecule.

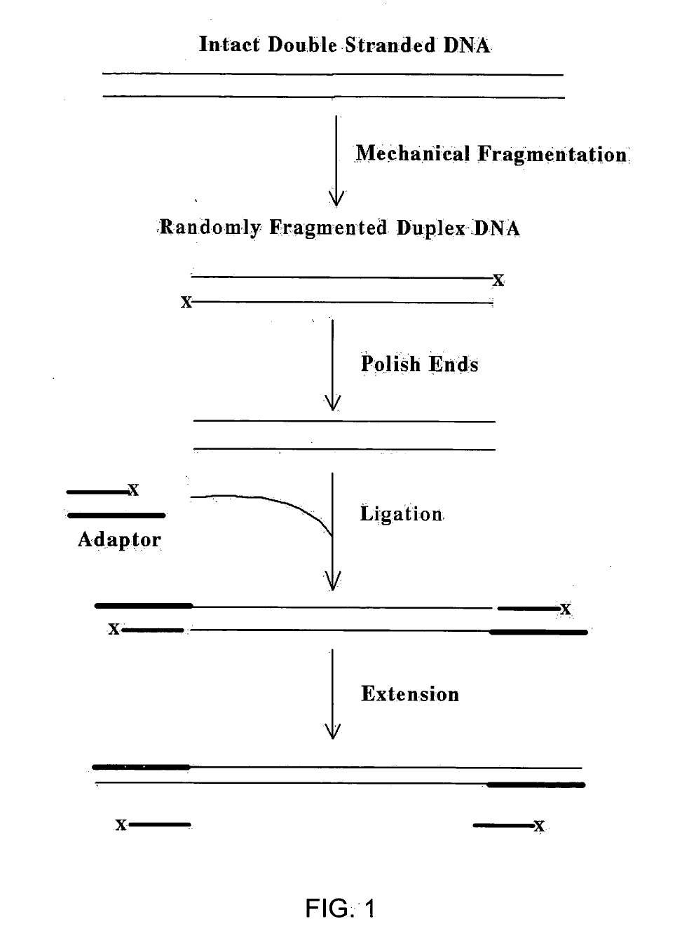

DNA that has been mechanically fragmented has been demonstrated to have blocked 3, ends that are incapable of being extended by Taq polymerase without a repair step. Furthermore, mechanical fragmentation utilizing a hydrodynamic shearing device (such as HydroShear; GeneMachines, Palo Alto, Calif.) results in at least three types of ends: 3′ overhangs, 5′ overhangs, and blunt ends. In order to effectively ligate the adaptors to these molecules and extend these molecules across the region of the known adaptor sequence, the 3′ ends need to be repaired so that preferably the majority of ends are blunt (FIG. 1). This procedure is carried out by incubating the DNA fragments with a DNA polymerase having both 3′ exonuclease activity and 3′ polymerase activity, such as Klenow or T4 DNA polymerase. Although reaction parameters may be varied by one of skill in the art, in an exemplary embodiment incubation of the DNA fragments with Klenow in the presence of 40 nmol dNTP and 1×T4 DNA ligase buffer results in optimal production of blunt end molecules with competent 3′ ends.

Alternatively, Exonuclease III and T4 DNA polymerase can be utilized to remove 3′ blocked bases from recessed ends and extend them to form blunt ends. In a specific embodiment, an additional incubation with T4 DNA polymerase or Klenow maximizes production of blunt ended fragments with 3′ ends that are competent to undergo ligation to the adaptor.

In specific embodiments, the ends of the double stranded DNA molecules still comprise overhangs following such processing, and particular adaptors are utilized in subsequent steps that correspond to these overhangs.

b. Repair of Chemically Fragmented DNA

Chemical fragmentation of DNA can be achieved by any method known in the art, including acid or alkaline catalytic hydrolysis of DNA (Richards and Boyer, 1965), hydrolysis by metal ions and complexes (Komiyama and Sumaoka, 1998; Franklin, 2001; Branum et al., 2001), hydroxyl radicals (Tullius, 1991; Price and Tullius, 1992) and/or radiation treatment of DNA (Roots et al., 1989; Hayes et al., 1990). Chemical treatment could result in double or single strand breaks, or both.

In a specific embodiment, chemical fragmentation occurs by heat. In a further specific embodiment, a temperature greater than room temperature, in some embodiments at least about 40° C., is provided. In alternative embodiments, the temperature is ambient temperature. In further specific embodiments, the temperature is between about 40° C. and 120° C., between about 80° C. and 100° C., between about 90° C. and 100° C., between about 92° C. and 98° C., between about 93° C. and 97° C., or between about 94° C. and 96° C. In some embodiments, the temperature is about 95° C.

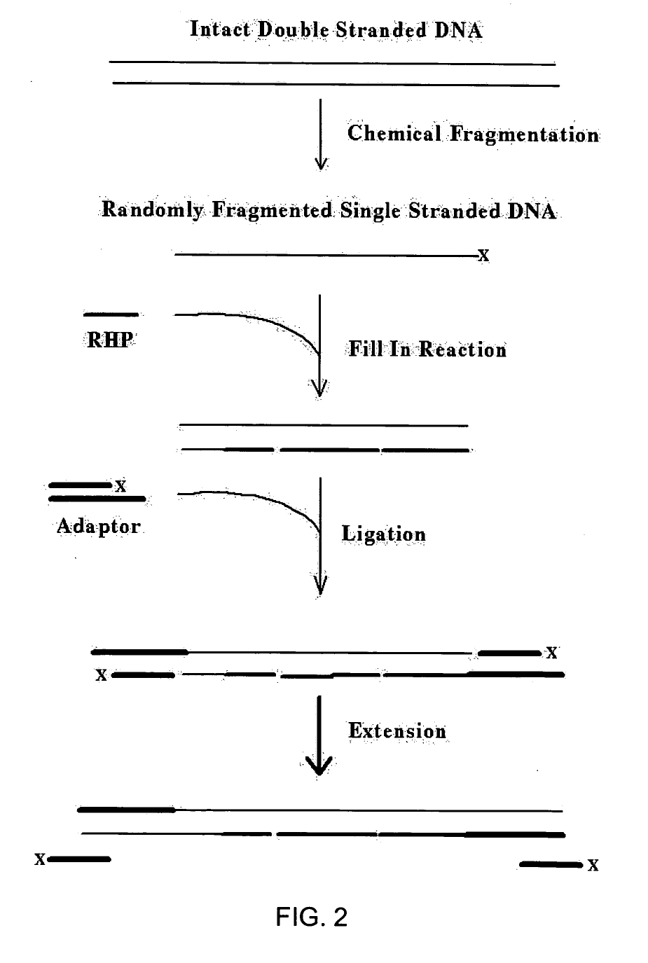

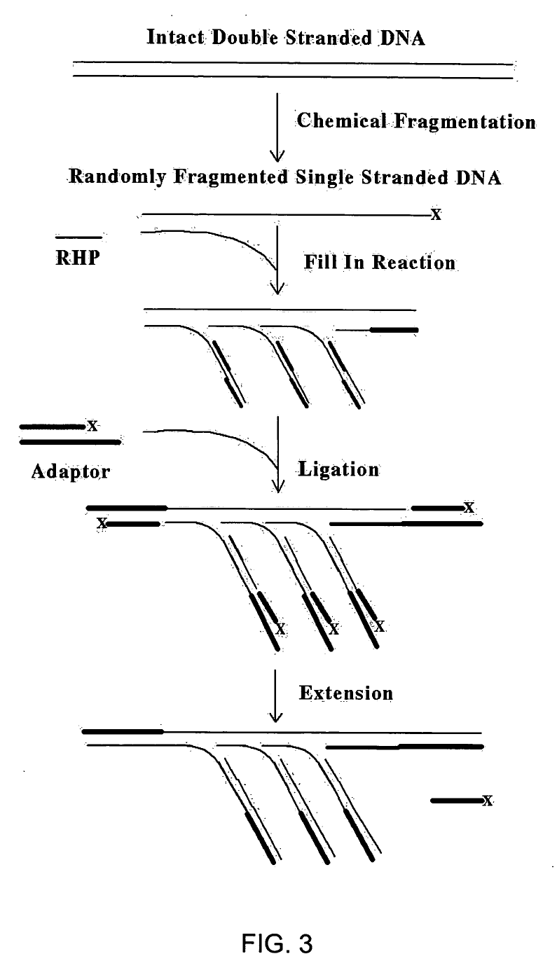

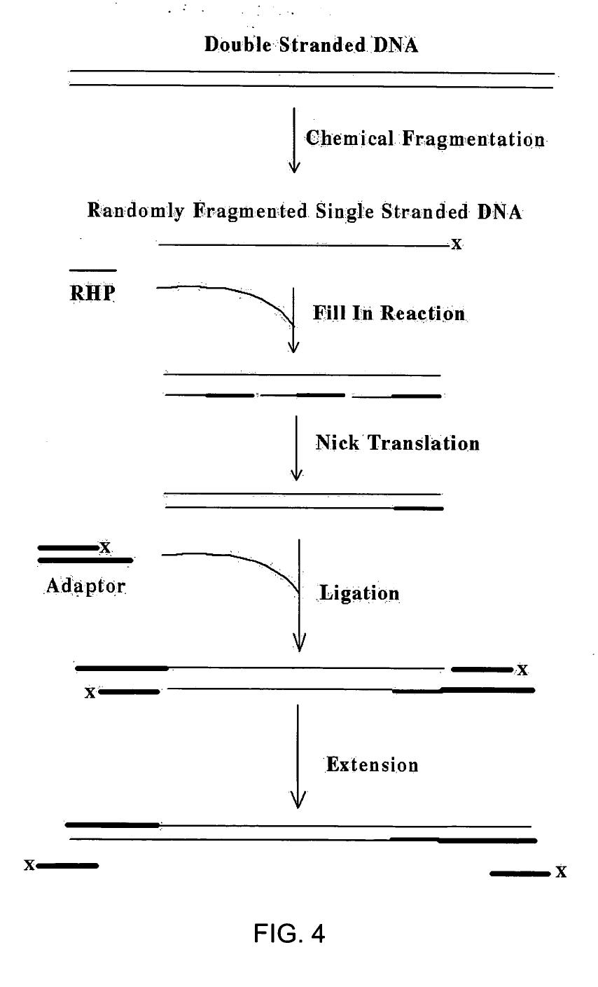

In a specific embodiment, DNA that has been chemically fragmented exists as single stranded DNA and has been demonstrated to have blocked 3′ ends. In order to generate double stranded 3′ ends that are competent to undergo ligation, a fill-in reaction with random primers and a DNA polymerase that has 3′-5′ exonuclease activity, such as Klenow, T4 DNA polymerase, or DNA polymerase I, is performed. This procedure will potentially result in several types of molecules depending on the polymerase used and the conditions of reaction. In the presence of a non strand-displacing polymerase, such as T4 DNA polymerase, fill-in with phosphorylated random primers will result in multiple short sequences that are extended until they are stopped by the presence of a downstream random-primed fragment. This will result in two ends that are competent to undergo ligation (FIG. 2). A strand-displacing enzyme such as Klenow will result in displacement of downstream fragments that can subsequently be primed and extended. This will result in production of a branched structure that has multiple ends competent to undergo ligation in the next step (FIG. 3). Finally, use of an enzyme with nick translation ability, such as DNA polymerase I, will result in nick translation of all fragments leading to a single secondary strand capable of ligation (FIG. 4). A skilled artisan recognizes that nick translation comprises a coupled polymerization/degradation process that is characterized by coordinated 5′-3′ DNA polymerase activity and 5′-3′ exonuclease activity. The two enzymes are usually present within one enzyme molecule (as in the case of Taq DNA polymerase or DNA polymerase I), however nick translation may also be achieved by simultaneous activity of multiple enzymes exhibiting separate polymerase and exonuclease activities. Incubation of the DNA fragments with Klenow in the presence of 0.1 to 10 pmol of phosphorylated primers in a two temperature protocol (37° C. and 12° C., for example) results in optimal production of blunt end fragments with 3′ ends that are competent to undergo ligation to the adaptor.

C. Repair of Enzymatically Fragmented DNA

Enzymatic fragmentation of DNA may be utilized by standard methods in the art, such as by partial restriction digestion by Cvi JI endonuclease (Gingrich et al., 1996), or by DNAse I (Anderson, 1981; Ausubel et al., 1987). Fragmentation by DNAse I may occur in the presence of Mg²⁺ ions (about 1-10 mM; predominantly single strand breaks) or in the presence of Mn²⁺ ions (about 1-10 mM; predominantly double strand breaks).

DNA that has been enzymatically fragmented in the presence of Mn²⁺ has been demonstrated to have either blunt ends or 1-2 bp overhangs. Thus, it is possible to omit the repair step and proceed directly to ligation of adaptors. Alternatively, the 3′ ends can be repaired so that a higher plurality of ends are blunt, resulting in improved ligation efficiency. This procedure is carried out by incubating the DNA fragments with a DNA polymerase containing both 3′ exonuclease activity and 3′ polymerase activity, such as Klenow or T4 DNA polymerase. For example, incubation of the DNA fragments with Klenow in the presence of 40 nmol dNTP and 1× T4 DNA ligase buffer results in optimal production of blunt end molecules with competent 3′ ends, although modifications of the reaction parameters by one of skill in the art are well within the scope of the invention.

Alternatively, Exonuclease III and T4 DNA polymerase can be utilized to remove 3′ blocked bases from recessed ends and extend them to form blunt ends. An additional incubation with T4 DNA polymerase or Klenow maximizes production of blunt ended fragments with 3′ ends that are competent to undergo ligation to the adaptor.

DNA that has been enzymatically digested with DNAse I in the presence of Mg²⁺ has been demonstrated to have single stranded nicks. Denaturation of this DNA would result in single stranded DNA fragments of random size and distribution. In order to generate double stranded 3′ ends, a fill in reaction with random primers and DNA polymerase that has 3′-5′ exonuclease activity, such as Klenow, T4 DNA polymerase, or DNA polymerase I, is performed. Use of these enzymes will result in the same types of products as described in item b —Repair of Chemically Fragmented DNA.

3. Sequence Attachment to the Ends of DNA Fragments

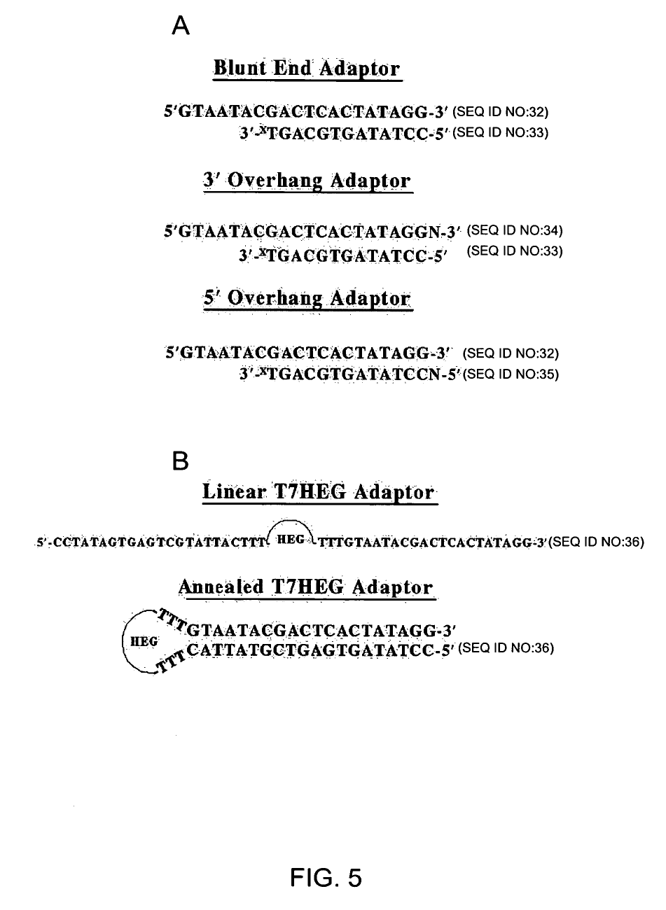

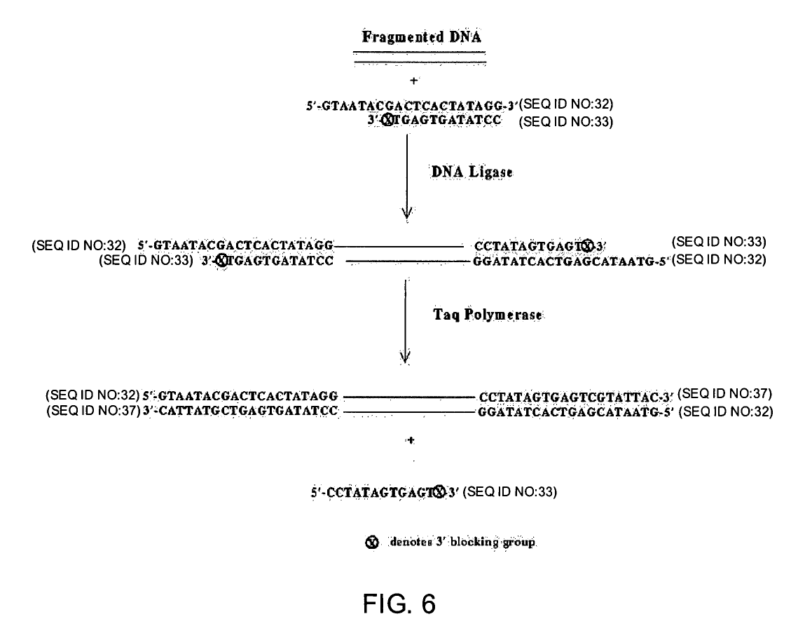

The following ligation procedure is designed to work with both mechanically and chemically fragmented DNA that has been successfully repaired and comprises blunt double stranded 3′ ends. Under optimal conditions, the repair procedures will result in the majority of products having blunt ends. However, due to the competing 3′ exonuclease activity and 3′ polymerization activity, there will also be a portion of ends that have about a 1 bp 5′ overhang or about a 1 bp 3′ overhang. Therefore, there are three types of adaptors that can be ligated to the resulting DNA fragments to maximize ligation efficiency, and preferably the adaptors are ligated to one strand at both ends of the DNA fragments. These three adaptors are illustrated in FIG. 5 and include: blunt end adaptor, 5′ N overhang adaptor, and 3′ N overhang adaptor. The combination of these 3 adaptors has been demonstrated to increase the ligation efficiency compared to any single adaptor. These adaptors are composed of two oligos, 1 short and 1 long, which are hybridized to each other at some region along their length. In a specific embodiment, the long oligo is a 20-mer that will be ligated to the 5′ end of fragmented DNA. In another specific embodiment, the short oligo strand is a 3, blocked 11-mer complementary to the 3′ end of the long oligo. A skilled artisan recognizes that the length of the oligos that comprise the adaptor may be modified, in alternative embodiments. For example, a range of oligo length for the long oligo is about 18 bp-about 100 bp, and a range of oligo length for the short oligo is about 7 bp-about 20 bp. Furthermore, the structure of the adaptors has been developed to minimize ligation of adaptors to each other via at least one of three means: 1) lack of a 5′ phosphate group necessary for ligation; 2) presence of about a 7 bp 5′ overhang that prevents ligation in the opposite orientation; and/or 3) a 3′ blocked base preventing fill-in of the 5′ overhang. The ligation of a specific adaptor is detailed in FIG. 6.

In a specific embodiment, there is an adaptor comprising a structure, such as a hairpin loop, that prevents undesirable modifications by the endonuclease and/or ligase in the mixture. In a further specific embodiment, there is a specific oligo (T7HEG adaptor; Integrated DNA Technologies; Coralville, Iowa) that is self-complementary and that will serve as a double stranded adaptor. The two complementary strands that normally comprise the adaptor are covalently joined by an 18 atom spacer (hexaethyleneglycol-based spacer; HEG) that is flexible enough to allow self-annealing of the complementary sequences, producing a blunt end adaptor sequence (FIG. 5B). The T7HEG oligo sequence (SEQ ID NO:36) is converted into the double stranded adaptor form by heating to 65° C. for 1 minute and then cooling to about room temperature.

In a specific embodiment, ligation of the adaptor occurs in the presence of 1×T4 DNA Ligase Buffer, 400 U T4 DNA Ligase, and 10 pmol each of blunt end, 5′ N overhang, and 3′ N overhang adaptors (FIG. 5A) and proceeds for 2 h at 16° C.

4. Combination of Polishing and Ligation Steps for 1 Step Repair and Ligation of Chemically Fragmented DNA

DNA that has been chemically fragmented often exists as single stranded DNA and has been demonstrated to have blocked 3′ ends. In order to generate double stranded 3′ ends that are competent to undergo ligation, a fill-in reaction is performed with random primers and DNA polymerase that has 3′-5′ exonuclease activity, such as Klenow. Addition of universal adaptors (FIG. 5A) or T7HEG adaptors (FIG. 5B) following the 37° C. 30′ incubation will allow the simultaneous polishing of the DNA fragment ends and ligation of the adaptors to these ends.

Alternatively, the adaptors may be added during the initial 37° C. step resulting in a 1 step reaction that is completed upon incubation at 16° C. A skilled artisan recognizes that a variety of different temperature protocols may be used to balance the random hexamer polymerization step with the polishing and ligation steps.

5. Extension of the 3′ End of the DNA Fragment to Fill in the Universal Adaptor

Due to the lack of a phosphate group at the 5′ end of the adaptor, only one strand of the adaptor (3′ end) will be covalently attached to the DNA fragment. A 72° C. extension step is performed on the DNA fragments in the presence of DNA polymerase, PCR Buffer, dNTP and universal primers. This step may be performed immediately prior to amplification using Taq polymerase, or may be carried out using a thermo-labile polymerase, such as if the libraries are to be stored for future use. The ligation and extension steps are detailed in FIG. 6.

6. Amplification of DNA Fragments using the Universal Primer

In a specific embodiment, the amplification reaction comprises about 1-5 ng of template DNA, Taq polymerase, dNTP, and T7 universal primer (5′-GTAATACGACTCACTATA-3′; SEQ ID NO: 11). In addition, fluorescein calibration dye (FCD) and SYBR Green I (SGI) may be added to the reaction to allow monitoring of the amplification using real-time PCR by methods well known in the art. PCR is carried out using a 2-step protocol of 94° C. 15″, 65° C. 2′ for the optimal number of cycles. Optimal cycle number is determined by analysis of DNA production using either real-time PCR or spectrophotometric analysis. Typically, about 5-15 μg of amplified DNA can be obtained from a 25-75 μl reaction using optimized conditions. The presence of the short oligo from the adaptor does not interfere with the amplification reaction due to its low melting temperature and the blocked 3′ end that prevents extension.

B. Generating DNA Fragment Libraries by Simultaneous Endonuclease Cleavage and Linker Ligation Reaction

In another aspect of the present invention, DNA fragment libraries are generated by concomitant endonuclease cleavage and linker ligation reactions, preferably in a single tube, a single reaction vessel, a single well, a single system, and preferably in the absence of any intermediate steps, such as DNA precipitation. Conversion of double-stranded DNA into libraries of smaller fragments has important applications for gene cloning, DNA sequence determination, and DNA amplification. Hybridization screening of genomic and cDNA fragments inserted into plasmid or bacteriophage vectors can identify novel genes homologous to the probe sequence and has led to the discovery of many important gene families within the same species, as well as homologs in different species. Shotgun sequencing of overlapping fragments of genomic libraries has proven to be an effective means of determining the entire genome sequence of numerous organisms and has also contributed to the identification of numerous single nucleotide polymorphisms. The simultaneous amplification of all fragments of a genomic library, or whole genome amplification, is critical for generating large amounts of material in cases where small genomic DNA quantities prevent large-scale genomic analysis.

Typically, libraries are generated in multiple steps, which include at least DNA fragmentation, repair/end polishing, and ligation. DNA fragmentation can be accomplished mechanically, by sonication or hydroshearing, chemically, and/or enzymatically using double-stranded DNA endonucleases such as deoxyribonuclease I (DNase I) or restriction endonucleases. DNA fragmentation by mechanical means can leave fragments with lengthy overhangs and non-phosphorylated 5′-termini or 3′-termini without hydroxyl groups that cannot be used for ligation. Thus, the ends of DNA fragmented by mechanical means are usually converted to blunt ends enzymatically, such as by the 5′-3′ polymerase activity and 3′-5, exonuclease activity of the Klenow fragment of E. coli DNA polymerase, and in specific embodiments comprises kinasing activity of T4 polynucleotide kinase. Enzymatic fragmentation produces 5′-phosphorylated and 3′-hydroxyl termini that can be ligated, but several different overhangs may be created that are usually converted to blunt ends by treatment with Klenow enzyme. Finally, the blunt-ended or end-repaired fragments are ligated to linkers or to a cloning vector in a separate ligation reaction.

Thus, the present invention overcomes a need in the art of providing high throughput library construction in the absence of multiple steps and the requirement for having to purify DNA between each step. The need for high throughput library construction is acute for large-scale genome sequencing projects and for amplifying thousands of clinical samples of limited quantity by whole genome amplification, and the present invention satisfies such a need.

1. Sources of DNA

The invention may be applied to any double-stranded DNA, including genomic DNA, cDNA, or fragments thereof.

2. Optimized Buffer for One-Step Reaction

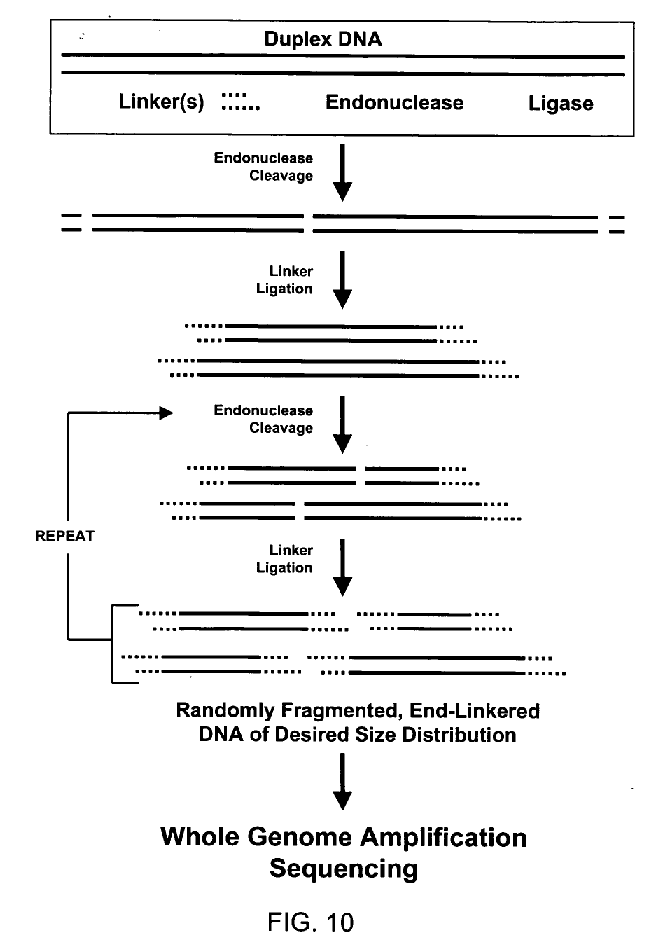

FIG. 10 illustrates the method of converting double-stranded DNA into a randomly fragmented, end-linkered library in a single reaction. The method relies on endonuclease cleavage and linker ligation occurring in the same reaction buffer. Over the course of time, the endonuclease repeatedly cleaves DNA into smaller fragments, while the ligase continually attaches linkers to the ends created by the cleavage. Since the buffer must support both endonuclease cleavage and ligation, a different combination of salt, pH, energy, and/or co-factor conditions must be established for each different combination of endonuclease and ligase. A skilled artisan is well aware of modifying reaction conditions to achieve the desired goal, based on current knowledge in the art and the teachings provided herein. It is preferable that a linker is ligated to a fragment end as soon as it is generated by endonuclease cleavage, so that at any time point during the reaction, the majority of the fragments will have linkers at both ends. Thus, if a buffer cannot be developed that supports both endonuclease cleavage and ligation effectively, it is preferable to develop a buffer that favors ligation efficiency over cleavage efficiency or to choose an endonuclease that functions in buffer conditions suited for ligation.

3. Choice of Endonucleases

The choice of endonuclease to be used in the reaction depends on several parameters, including at least the choice of ligase, reaction temperature, and/or downstream application of the library. The most commonly used enzyme for ligation, T4 DNA ligase, has optimal activity at 16° C.-25° C. and requires ATP, DTT, and Mg²⁺ or Mn²⁺ divalent cations for catalytic activity. Depending on the downstream library application, different average fragment sizes may be desired. For sequencing or cloning applications, it may be desirable to have an average fragments size of >about 5 kilobases. If the linkered DNA fragments will be amplified by polymerase chain reaction (PCR), smaller fragment sizes might be desired. By using endonucleases with no or short DNA sequence specificities, it would be possible to generate both large and short average fragment size libraries by controlling the extent of cleavage. These endonucleases also can generate a library of randomly overlapping fragments of the genome, which increases the probability of obtaining the greatest coverage for shotgun sequencing and for amplifying all genomic regions with similar efficiency for whole genome amplification.

Thus, in a preferred embodiment, endonucleases are utilized that function at about 16° C.-about 25° C., function in the presence of ATP, DTT, Mg²⁺, and/or Mn²⁺, and cleave in a sequence-independent manner or with short (about 2 to about 4 base pairs) DNA sequence specificities. Nonlimiting examples of endonucleases that satisfy such parameters include deoxyribonuclease I (DNase I) and the Cvi family of endonucleases produced by the Chlorella virus.

The Cvi family of endonucleases comprises at least CviJI and CviTI. CviJI may be obtained from CHIMERx (Madison, Wis.) and EURxLtd (Gdansk, Poland). The recognition site for CviJI is RG\hat{ }CY (average frequency is about 64 bases). CHIMERx also sells another version called CviJI*. Under “relaxed” conditions (in the presence of Mg²⁺ and ATP), CviJI* cleaves the sequence 5′-GC-3′ except 5′-YGCR-3′ (like a 2-3 base recognition site). The isoschizomer of this enzyme is CviTI (Megabase Research Products; Lincoln, Nebr.). Another version of the same enzyme, CviTI* (like CviJI*, it also has a different buffer) has the specificity NR\hat{ }YN (average frequency is about 16 bases).

4. Design of Linkers

An important feature of the invention is that a linker (which may also be referred to herein as an adaptor) or mixture of linkers is utilized that can be ligated to every predicted fragment end produced by endonuclease digestion but that cannot form linker-linker dimers. It is also preferable to design the linkers such that they are not themselves susceptible to cleavage by the endonuclease. For endonucleases with sequence specificities, the linkers are designed such that the duplex region of the linkers does not comprise the recognition sequence(s) for the endonuclease. When using sequence-independent endonucleases, some cleavage of linkers will occur, but that effect can be overcome by adding a large molar excess of linkers to the reaction.

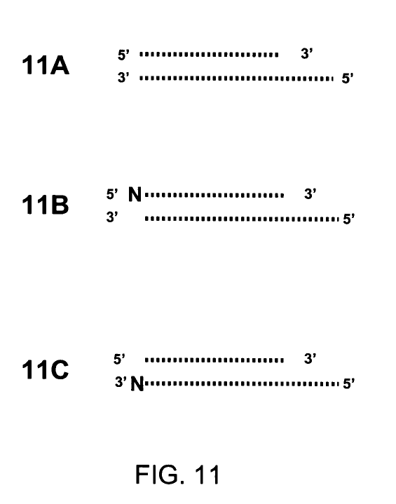

A critical feature of the linkers is that neither complementary oligonucleotide comprising the linker has a 5′-phosphate group (FIG. 11). The end of the linker that will be attached to the fragment end has a 3′-hydroxyl group, but the other end is not required to have a 3′-hydroxyl group. Since the ligation-competent end of the linkers has a 3′-hydroxyl on one strand but no 5′-phosphate on the other strand, it is not possible to form linker-linker dimers. On the other hand, the strand of duplex genomic DNA fragments that has a 5′-phosphate group may be ligated to the strand of linker that has the 3′-hydroxyl group.

Three kinds of linkers can be designed that represent all possible fragment ends created by endonucleases. The first kind of linker, illustrated in FIG. 11A, is designed for ligation to blunt-ended DNA fragments. The second kind of linker, illustrated in FIG. 11B, is designed for ligation to DNA fragments with 5′ overhangs. The number of overhanging bases on the 5′ end of the shorter linker oligonucleotide corresponds to the number of bases on the 5′ overhang of the DNA fragments. Each overhang base on the linker oligonucleotide can correspond to a single nucleotide or any combination of the four nucleotides, A, C, G, and T that can base pair with the predicted DNA fragment overhang. The third kind of linker, illustrated in FIG. 11C, is designed for ligation to DNA fragments with 3′ overhangs. The composition of these linkers is similar to those described above in FIG. 11B, except that the overhanging bases are on the 3′ end of the longer linker oligonucleotide.

5. Reaction Conditions

A critical feature of the method is to balance the kinetics of linker ligation with the kinetics of endonuclease cleavage. If the endonuclease cleavage to the desired average fragment size occurs more rapidly than ligation can occur, most of the fragments will not have linkers at both ends. Thus, it is desirable to use endonuclease concentrations that will cleave to the desired average fragment size over the course of several hours. This is particularly important when cleavage produces blunt ends, since blunt end ligation kinetics are slow compared to cohesive end ligation. It is also important to use a large molar excess of linkers (>about 50-fold) to the predicted number of fragment ends so that linker ligation to the ends is more efficient than end to end ligation, to minimize the number of longer, chimeric fragments. Because linker ligation and endonuclease cleavage are occurring in the same reaction over time, it is possible to generate multiple libraries of differing average fragment size by withdrawing aliquots of the same reaction at different incubation times.

III. Nucleic Acids

In a specific embodiment, the method of the present invention comprises amplification of at least one nucleic acid. The term “nucleic acid” or “polynucleotide” will generally refer to at least one molecule or strand of DNA, or a derivative or analog thereof, comprising at least one nucleobase, such as, for example, a naturally occurring purine or pyrimidine base found in DNA (e.g. adenine “A,” guanine “G,” thymine “T” and cytosine “C”). The term “nucleic acid” encompasses the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, by the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards a target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and analogs. Non-limiting examples of nucleobases include purines and pyrimidines, as well as derivatives and analogs thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or analogs of a naturally occurring 5-carbon sugar or phosphorus moiety, and non-limiting examples are described herein.

IV. Amplification of Nucleic Acids

Nucleic acids useful as templates for amplification are generated by methods described herein. In a specific embodiment, the DNA molecule from which the methods generate the nucleic acids for amplification may be isolated from cells, tissues or other samples according to standard methodologies (Sambrook et al., 1989).

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

Extension of the hybridized primer pairs occurs under conditions suitable for the DNA polymerase. In some instances, hybridization and extension are carried out at the same temperature, while in other cases, hybridization occurs at a temperature optimal for the primers while extension occurs at a temperature optimal for the polymerase. The length of the extension step can be varied depending on the size of the products being produced. Increasing the extension time will result in the production of longer fragments. In contrast, a shorter time of extension can be utilized to select for shorter products only. One skilled in the art will realize that the variation of the extension time can be utilized to select for different size products and that this variation can be used to improve amplification of products of the desired length.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 37-72° C.) and a daughter strand extended from the primers (72° C.). As the daughter strands are created they act as templates in subsequent cycles. Thus, the template region between the two primers is amplified exponentially, rather than linearly.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR™ are described in U.S. Pat. No. 5,882,864.

LCR

Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

C. Qbeta Replicase

Qbeta Replicase, described in PCT Patent Application No. PCT/US87/00880, also may be used as still another amplification method in the present invention. In this method, a replicative sequence of RNA that has a region complementary to that of a target is added to a sample in the presence of an RNA polymerase. The polymerase will copy the replicative sequence that can then be detected.

D. Isothermal Amplification

An isothermal amplification method, in which restriction endonucleases and ligases are used to achieve the amplification of target molecules that contain nucleotide thiophosphates in one strand of a restriction site also may be useful in the amplification of nucleic acids in the present invention. Such an amplification method is described by Walker et al. 1992, incorporated herein by reference.

E. Strand Displacement Amplification

Strand Displacement Amplification (SDA) is another method of carrying out isothermal amplification of nucleic acids that involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

F. Cyclic Probe Reaction

Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

G. Transcription-Based Amplification

Other nucleic acid amplification procedures include transcription-based amplification systems (TAS), including nucleic acid sequence based amplification (NASBA) and 3SR (Kwoh et al., 1989; PCT Patent Application WO 88/10315), each incorporated herein by reference).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase, such as T7 or SP6. In an isothermal cyclic reaction, the RNAs are reverse transcribed into double stranded DNA, and transcribed once again with an RNA polymerase, such as T7 or SP6. The resulting products, whether truncated or complete, indicate target specific sequences.

H. Rolling Circle Amplification

Rolling circle amplification (U.S. Pat. No. 5,648,245) is a method to increase the effectiveness of the strand displacement reaction by using a circular template. The polymerase, which does not have a 5′ exonuclease activity, makes multiple copies of the information on the circular template as it makes multiple continuous cycles around the template. The length of the product is very large—typically too large to be directly sequenced. Additional amplification is achieved if a second strand displacement primer is added to the reaction using the first strand displacement product as a template.

I. Other Amplification Methods

Other amplification methods, as described in British Patent Application No. GB 2,202,328, and in PCT Patent Application No. PCT/US89/01025, each incorporated herein by reference, may be used in accordance with the present invention. In the former application, ““modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “RACE” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention, Wu et al., 1989, incorporated herein by reference).

V. Restriction Endonucleases

In a preferred embodiment, a DNA molecule is fragmented randomly, such as by mechanical, chemical, and/or enzymatic fragmentation (such as with DNAse I). In an alternative embodiment, a restriction endonuclease is utilized to fragment the DNA.

Restriction endonucleases (restriction enzymes) recognize specific short DNA sequences four to eight nucleotides long (see Table I), and cleave the DNA at a site within this sequence. In the context of the present invention, restriction enzymes are used to cleave DNA molecules at sites corresponding to various restriction-enzyme recognition sites. In some embodiments, frequently cutting enzymes, such as the four-base cutter enzymes, are utilized, as this yields DNA fragments that are in the right size range for subsequent amplification reactions. Some of the preferred four-base cutters are NlaIII, DpnII, Sau3AI, Hsp92II, MboI, NdeII, Bsp1431, Tsp509 I, HhaI, HinP1I, HpaII, MspI, Taq alphaI, MaeII or K2091. In a preferred embodiment a restriction enzyme that generates a blunt end is utilized.

As the sequence of the recognition site is known (see Table I), primers can be designed comprising nucleotides corresponding to the recognition sequences. If the primer sets have in addition to the restriction recognition sequence, degenerate sequences corresponding to different combinations of nucleotide sequences, one can use the primer set to amplify DNA fragments that have been cleaved by the particular restriction enzyme. Table I exemplifies the currently known restriction enzymes that may be used in the invention.

TABLE I
RESTRICTION ENZYMES

| | |
|---|---|
| Enzyme Name | Recognition Sequence |

| | |
|---|---|
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| | (SEQ ID NO:14) |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| | (SEQ ID NO:15) |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| | (SEQ ID NO:16) |
| BciV I | GTATCC |
| Bcl I | TGATCA |
| Bfa I | CTAG |
| Bgl I | GCCNNNNNGGC |
| | (SEQ ID NO:17) |
| Bgl II | AGATCT |
| Blp I | GCTNAGC |
| Bmr I | ACTGGG |
| Bpm I | CTGGAG |
| BsaA I | YACGTR |
| BsaB I | GATNNNNATC |
| | (SEQ ID NO:18) |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| | (SEQ ID NO:19) |
| BsmA I | GTCTC |
| BsmB I | CGTCTC |
| BsmF I | GGGAC |
| Bsm I | GAATGC |
| BsoB I | CYCGRG |
| Bsp1286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAP I | GCANNNNNTGC |
| | (SEQ ID NO:20) |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| | (SEQ ID NO:21) |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dde I | CTNAG |
| Dpn I | GATC |
| Dpn II | GATC |
| Dra I | TTTAAA |
| Dra III | CACNNNGTG |
| Drd I | GACNNNNNNGTC |
| | (SEQ ID NO:22) |
| Eae I | YGGCCR |
| Eag I | CGGCCG |
| Ear I | CTCTTC |
| Eci I | GGCGGA |
| EcoN I | CCTNNNNNAGG |
| | (SEQ ID NO:23) |
| EcoO109 I | RGGNCCY |
| EcoR I | GAATTC |
| EcoR V | GATATC |
| Fau I | CCCGCNNNN |
| Fnu4H I | GCNGC |
| Fok I | GGATG |
| Fse I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind III | AAGCTT |
| Hinf I | GANTC |
| HinP1 I | GCGC |
| Hpa I | GTTAAC |
| Hpa II | CCGG |
| Hph I | GGTGA |
| Kas I | GGCGCC |
| Kpn I | GGTACC |
| Mbo I | GATC |
| Mbo II | GAAGA |
| Mfe I | CAATTG |
| Mlu I | ACGCGT |
| Mly I | GAGTCNNNNN |
| | (SEQ ID NO:24) |
| Mnl I | CCTC |
| Msc I | TGGCCA |
| Mse I | TTAA |
| Msl I | CAYNNNNRTG |
| | (SEQ ID NO:25) |
| MspA1 I | CMGCKG |
| Msp I | CCGG |
| Mwo I | GCNNNNNNNGC |
| | (SEQ ID NO:26) |
| Nae I | GCCGGC |
| Nar I | GGCGCC |
| Nci I | CCSGG |
| Nco I | CCATGG |
| Nde I | CATATG |
| NgoMI V | GCCGGC |
| Nhe I | GCTAGC |
| Nla III | CATG |
| Nla IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| | (SEQ ID NO:27) |
| Ple I | GAGTC |
| Pme I | GTTTAAAC |
| Pml I | CACGTG |
| PpuM I | RGGWCCY |
| PshA I | GACNNNNGTC |
| | (SEQ ID NO:28) |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| ScrF I | CCNGG |
| SexA I | ACCWGGT |
| SfaN I | GCATC |
| Sfc I | CTRYAG |
| Sfi I | GGCCNNNNNGGCC |
| | (SEQ ID NO:29) |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Taq I | TCGA |
| Tfi I | GAWTC |
| Tli I | CTCGAG |
| Tse I | GCWGC |
| Tsp45 I | GTSAC |
| Tsp509 I | AATT |
| TspR I | CAGTG |
| Tth111 I | GACNNNGTC |
| Xba I | TCTAGA |
| Xcm I | CCANNNNNNNNNTGG |
| | (SEQ ID NO:30) |
| Xho I | CTCGAG |
| Xma I | CCCGGG |
| Xmn I | GAANNNNTTC |
| | (SEQ ID NO:31) |

In a preferred embodiment, a restriction endonuclease of the Cvi family (from the Chlorella virus) is utilized in methods of the present invention.

Other Enzymes

Other enzymes that may be used in conjunction with the invention include nucleic acid modifying enzymes are listed in Tables II and III.

TABLE II
POLYMERASES AND REVERSE TRANSCRIPTASES

Thermostable DNA Polymerases:
OmniBase ™ Sequencing Enzyme
Pfu DNA Polymerase
Taq DNA Polymerase
Taq DNA Polymerase, Sequencing Grade
TaqBead ™ Hot Start Polymerase
AmpliTaq Gold
Tfl DNA Polymerase
Tli DNA Polymerase
Tth DNA Polymerase
DNA Polymerases:
DNA Polymerase I, Klenow Fragment, Exonuclease Minus
DNA Polymerase I
DNA Polymerase I Large (Klenow) Fragment
Terminal Deoxynucleotidyl Transferase
T4 DNA Polymerase
Reverse Transcriptases:
AMV Reverse Transcriptase
M-MLV Reverse Transcriptase

TABLE III
DNA/RNA MODIFYING ENZYMES

Ligases:
T4 DNA Ligase
Kinases
T4 Polynucleotide Kinase
Isomerase
Topoisomerase I

VI. DNA Polymerases

In some embodiments, it is envisioned that the methods of the invention could be carried out with one or more enzymes where multiple enzymes combine to carry out the function of a single DNA polymerase molecule retaining 5′-3′ exonuclease activity. Effective polymerases that retain 5′-3′ exonuclease activity include, for example, E. coli DNA polymerase I, Taq DNA polymerase, S. pneumoniae DNA polymerase I, Tfl DNA polymerase, D. radiodurans DNA polymerase I, Tth DNA polymerase, Tth XL DNA polymerase, M.tuberculosis DNA polymerase I, M thermoautotrophicum DNA polymerase I, Herpes simplex-i DNA polymerase, E. coli DNA polymerase I Klenow fragment, Vent DNA polymerase, thermosequenase and wild-type or modified T7 DNA polymerases. In preferred embodiments, the effective polymerase is E. coli DNA polymerase I, Klenow, or Taq DNA polymerase.

Where a break in the substantially double stranded nucleic acid template is a gap of at least a base or nucleotide in length that comprises, or is reacted to comprise, a 3′ hydroxyl group, the range of effective polymerases that may be used is even broader. In such aspects, the effective polymerase may be, for example, E. coli DNA polymerase I, Taq DNA polymerase, S. pneumoniae DNA polymerase I, Tfl DNA polymerase, D. radiodurans DNA polymerase I, Tth DNA polymerase, Tth XL DNA polymerase, M tuberculosis DNA polymerase I, M thermoautotrophicum DNA polymerase I, Herpes simplex-i DNA polymerase, E. coli DNA polymerase I Klenow fragment, T4 DNA polymerase, Vent DNA polymerase, thermosequenase or a wild-type or modified T7 DNA polymerase. In preferred aspects, the effective polymerase is E. coli DNA polymerase I, M tuberculosis DNA polymerase I, Taq DNA polymerase, or T4 DNA polymerase.

VII. Hybridization

Depending on the application envisioned, one would desire to employ varying conditions of hybridization to achieve varying degrees of selectivity of the probe or primers for the target sequence, such as in the adaptor. For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 35 mM MgCl₂, and 1.0 mM dithiothreitol, at temperatures between approximately 20° C. to about 37° C. Other hybridization conditions utilized could include approximately 10 mM Tris-HCl (pH 8.3), 50 mM KCl, and 1.5 mM MgCl₂, at temperatures ranging from approximately 40° C. to about 72° C.

VIII. DNA Archiving, Storage, Retrieval, and Re-Amplification

Genomic libraries containing a pool of randomly generated overlapping DNA fragments with short universal sequence at both ends provide a very efficient resource for highly representative whole genome amplification. The size (about 200-2,000 bp) and presence of a universal priming site make them also very attractive for such applications as DNA archiving, storing, retrieving and/or re-amplifying. Multiple libraries can be immobilized and stored as micro-arrays. Libraries covalently attached by one end to the bottom of tubes, micro-plates or magnetic beads, for example, can be used many times by replicating immobilized amplicons, dissociating replicated molecules for immediate use, and returning the original immobilized WGA library for continuing storage.

The structure of WGA amplicons can also be easily modified to introduce a personal identification (ID) DNA tag to the genomic sample to prevent an unauthorized amplification and use of DNA. Only those who know the sequence of the ID tag will be able to amplify and analyze genetic material. The tags can be also useful for preventing genomic cross-contaminations when dealing with many clinical DNA samples. Also, WGA libraries created from large bacterial clones (BACs, PACs, cosmids, etc.) can be amplified and used to produce genomic micro-arrays.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Whole Genome Amplification of Human Genomic DNA Fragmented by Mechanical Methods

This example, illustrated in FIG. 1, describes the amplification of genomic DNA that has been fragmented to an average size of 1.5 kb using mechanical methods, specifically hydrodynamic shearing (HydroShear, Gene Machines; Palo Alto, Calif.).

Aliquots of 110 μl of DNA prep containing 50 ng to 10 μg of DNA were heated to 65° C. for 2′, vortexed for 15″ and incubated for an additional 2′ at 65° C. The samples were spun at 12 min at RT at 16,000×G. One hundred jl of sample was transferred to a new tube and subjected to mechanical fragmentation on a HydroShear device (Gene Machines) for 20 passes at a speed code of 3, following the manufacturer's protocol. The sheared DNA has an average size of 1.5 kb as predicted by the manufacturer and confirmed by gel electrophoresis. To prevent carry-over contamination, the shearing assembly of the HydroShear was washed 3 times each with 0.2 M HCl, and 0.2 M NaOH, and 5 times with TE-L buffer prior to and following fragmentation. All wash solutions were 0.2 μm filtered prior to use.

Fragmented DNA samples may be used immediately for library preparation or stored at −20° C. prior to use. The first step of this embodiment of library preparation is to repair the 3′ end of all DNA fragments and to produce blunt ends. This step comprises incubation with at least one polymerase. Specifically, 11.5 μl 10× T4 DNA ligase buffer, 0.38 μl dNTP (mM FC), 0.46 μl Klenow (2.3 U, USB) and 2.66 μl H₂O were added to the 100 μl of fragmented DNA. The reaction was carried out at 25° C. for 15′, and the polymerase was inactivated at 75° C. for 15′ and then chilled to 4° C.

Universal adaptors were ligated to the 5′ ends of the DNA using T4 DNA ligase by addition of 4 μl T7 adaptors (10 pmol each of the blunt end, 5′ N overhang, and 3′ N overhang adaptors) and 1 μl T4 DNA Ligase (2,000 U). The reaction was carried out for 1 h at 16° C. and then held at 4° C. until use. Alternatively, the libraries can be stored at −20° C. for extended periods prior to use.

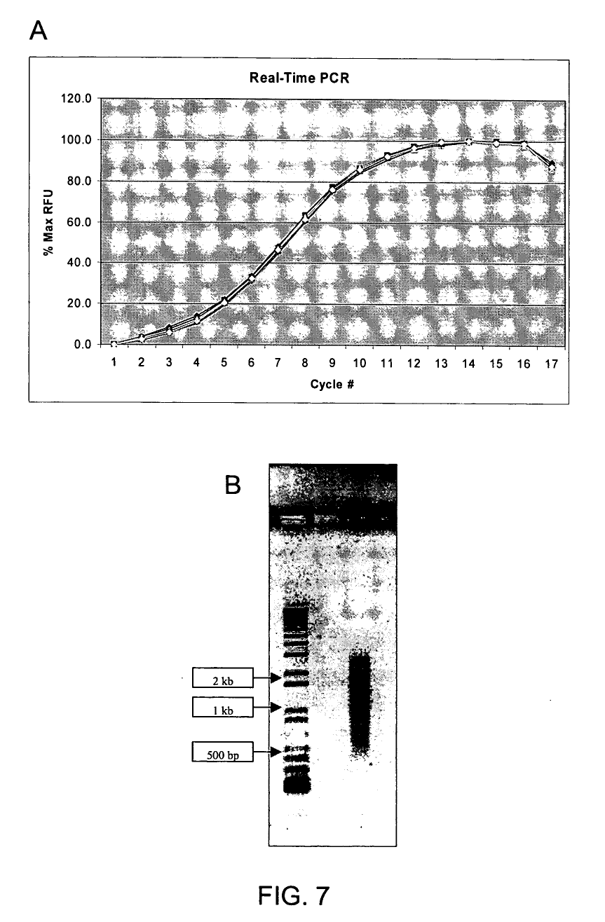

Extension of the 3′ end to fill in the universal adaptor and subsequent amplification of the library were carried out under the same conditions. Five nanograms (ng) of library is added to a 75 μl reaction comprising 25 pmol T7 universal primer (SEQ ID NO:11), 120 nmol dNTP, 1×PCR Buffer (Clontech), 1× Titanium Taq. Fluorescein calibration dye (1:100,000) and SYBR Green SGI (1:100,000) are also added to allow monitoring of the reaction using the I-Cycler Real-Time Detection System (Bio-Rad). The samples are initially heated to 75° C. for 15′ to allow extension of the 3′ end of the fragments to fill in the universal adaptor sequence and displace the short, blocked fragment of the universal adaptor. Subsequently, amplification is carried out by heating the samples to 95° C. for 3′30″, followed by 14-19 cycles of 94° C. 15″, 65° C. 2′. The cycle number is dependent on the amount of template in the reaction. Typically, for 5 ng of library the optimal number of cycles is about 17 (FIG. 7A). Analysis of DNA production has indicated that there is a continual increase in DNA through cycle 17. At cycles 18 and later, there is an apparent plateau of DNA production by spectrophotometric analysis. However, there is a decrease in competent DNA when specific sites are analyzed by quantitative real-time PCR.

Following amplification, the DNA samples were purified using the Qiaquick kit (Qiagen) and quantitated. In order to demonstrate the ability of these libraries to be amplified multiple times without loss of representation, 5 ng aliquots of the purified, amplified product were subjected to a secondary amplification reaction. Specifically, 5 ng of library is added to a 75 μl reaction comprising 25 pmol T7 universal primer (SEQ ID NO:11), dNTP, 1×PCR Buffer (Clontech), 1× Titanium Taq. Fluorescein calibration dye (1:100,000) and SYBR Green I (1:100,000) are also added to allow monitoring of the reaction using real-time PCR (Bio-Rad). Amplification is carried out by heating the samples to 95° C. for 3′30”, followed by 10-19 cycles of 94° C. 15″, 65° C. 2′. The cycle number is dependent on the amount of template in the reaction. Typically, for 5 ng of library the optimal number of cycles is 14 for a secondary amplification. Analysis of DNA production has indicated that there is a continual increase in DNA through about cycle 14. At about cycles 15 and later, there is an apparent plateau of DNA production by spectrophotometric analysis. However, there is a decrease in competent DNA when specific sites are analyzed by quantitative real-time PCR. It should also be noted that the 15′ 75° C. extension step utilized in the primary amplification reaction following library construction is not necessary for subsequent rounds of amplification due to the fact that the 3′ ends of the adaptor sequence already filled in.

The amplified material was purified by Qiagen's Qiaquick kit and quantified spetrophotometrically. Gel analysis of the amplified products (FIG. 7B) indicated a size distributed 500 bp to 3 kb) similar to the original, hydrosheared DNA. Additionally, the amplified DNA was analyzed using real-time, quantitative PCR using a panel of 103 human genomic STS markers. The markers that make up the panel are listed in Table IV. Quantitative Real-Time PCR was performed using an I-Cycler Real-Time Detection System (Bio-Rad), as per the manufacturer's directions. Briefly, 25 μl reactions were amplified for 40 cycles at 94° C. for 15 sec and 65° C. for 1 min. Standards corresponding to 10, 1, and 0.2 ng of fragmented DNA were used for each STS, quantities were calculated by standard curve fit for each STS (1-Cycler software, Bio-Rad) and were plotted as frequency histograms.

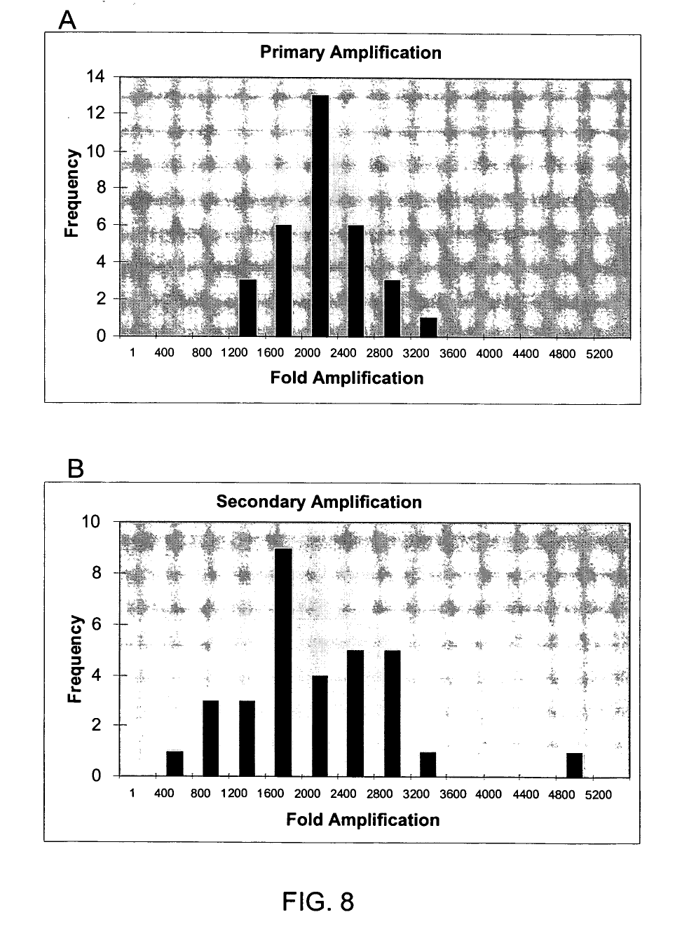

Quantitative real-time PCR demonstrated that 90% of the 103 markers were within a factor of 2 of the mean amplification for both the primary and secondary WGA products. Furthermore, all sites tested were detected, indicating that no sequences were lost during library preparation and amplification. FIG. 8 is a histogram of the representation of the 103 human genomic STS markers in the amplified DNA of one sample from both a primary (FIG. 8A) and a secondary (FIG. 8B) amplification. These results indicate that there is no significant decrease in the representation of specific loci following multiple rounds of amplification and demonstrates that the creation of the amplified products using the described method has resulted in DNA Immortalization.

TABLE IV
EXEMPLARY HUMAN STS MARKERS USED FOR
REPRESENTATION ANALYSIS BY QUANTITATIVE
REAL-TIME PCR

| No* | UniSTS Database Name** |
|---|---|
| 1 | RH18158 |
| 2 | SHGC-100484 |
| 3 | SHGC-82883 |
| 4 | SHGC-149956 |
| 5 | SHGC-146783 |
| 6 | SHGC-102934 |
| 8 | csnpmnat1-pcr1-1 |
| 9 | stSG62224 |
| 10 | SHGC-142305 |
| 12 | SHGC-80958 |
| 13 | SHGC-74059 |
| 14 | SHGC-83724 |
| 16 | SHGC-145896 |
| 19 | SHGC-155401 |
| 20 | csnpharp-pcr2-3 |
| 22 | stb39J12.sp6 |
| 23 | SHGC-149127 |
| 26 | 949_F_8Left |
| 29 | SHGC-148759 |
| 30 | SHGC-154046 |
| 31 | WI-19180 |
| 35 | SHGC-146602 |
| 36 | SHGC-130262 |
| 38 | SHGC-130314 |
| 40 | SHGC-147491 |
| 41 | stSG53466 |
| 42 | SHGC-105883 |
| 43 | SHGC-79237 |
| 44 | SHGC-153761 |
| 46 | stSG50529 |
| 47 | SHGC-132199 |
| 49 | stSG49452 |
| 51 | SGC32543 |
| 52 | SHGC-2457 |
| 53 | stSG53950 |
| 54 | stSG43297 |
| 55 | SHGC-81536 |
| 58 | stSG48086 |
| 60 | stSG62388 |
| 62 | stSG50542 |
| 63 | stSG44393 |
| 66 | SHGC-9458 |
| 67 | SHGC-5506 |
| 68 | SHGC-153324 |
| 69 | stSG53179 |
| 70 | sts-X16316 |
| 71 | stSG51782 |
| 72 | stSG48421 |
| 74 | stGDB: 442878 |
| 76 | WI-6290 |
| 77 | T94852 |
| 79 | SHGC-11640 |
| 80 | H58497 |
| 81 | stSG34953 |
| 82 | KIAA0108 |
| 83 | Y00805 |
| 84 | sts-W93373 |
| 85 | stSG45551 |
| 86 | U34806 |
| 88 | SHGC-12728 |
| 89 | SHGC-10570 |
| 91 | stSG52141 |
| 92 | SHGC-58853 |
| 94 | SHGC-36464 |
| 96 | stSG8946 |
| 97 | SHGC-10187 |
| 99 | WI-13668 |
| 103 | stSG49584 |
| 104 | M55047 |
| 105 | SHGC-102231 |
| 106 | stSG60168 |
| 107 | stSG50880 |
| 108 | stSG39197 |
| 110 | sts-AA035504 |
| 111 | SGC35140 |
| 113 | stSG53011 |
| 114 | sts-R44709 |
| 116 | SHGC-149512 |
| 117 | stSG55021 |
| 118 | SHGC-79529 |
| 119 | KIAA0181 |
| 120 | SHGC-105119 |
| 121 | SHGC-79242 |
| 122 | SHGC-170363 |
| 123 | stSG50637 |
| 126 | RH69540 |
| 130 | GDB: 181552 |
| 133 | 1770 |
| 134 | 1314 |
| 135 | SHGC-104164 |
| 136 | SHGC-101034 |
| 137 | stSG62239 |
| 138 | stSG60144 |
| 139 | stSG58407 |
| 140 | stSG58405 |
| 141 | sts-T50718 |
| 144 | SHGC-17057 |
| 145 | sts-N90764 |

*Omitted sequential numbers indicate dropped STS sequences that did not amplify well in quantitative RT-PCR
**Unique names of STS marker sequences from the National Center for Biotechnology Information UniSTS database. Sequences of the STS regions as well as the forward and backward primers used in quantitative real-time PCR can be found in the UniSTS database at the National Center for Biotechnology Information's website.

### Example 2

### Whole genome amplification of human genomic DNA (1 μg Template) Fragmented by Chemical Methods

This example describes the amplification of 1 μg of genomic DNA that has been fragmented to an average size of 1 kb using chemical methods, specifically thermal fragmentation.

Human DNA (1 μg) was diluted to 100 ng/μl in TE (10 mM Tris, 1 mM EDTA, pH 7.5). DNA was subsequently heated to 95° C. for 4′, and then cooled to 4° C. Thirty microliters of TE was added to the DNA to yield a concentration of 25 ng/μl. Four microliters (100 ng) of DNA was then added to 6 μl H₂O and 2 μl 10×T4 DNA Ligase Buffer (NEB) and the mixture was heated to 95° C. for 10′, and then cooled to 4° C.

In order to generate competent ends for ligation, 40 nmol dNTP (Clontech), 10 pmol phosphorylated random hexamer primers (Genelink), and 5 U Klenow (NEB) were added resulting in a final volume 15 μl, and the reaction was incubated at 37° C. for 30′ and 12° C. for 1 h. Following incubation, the reaction was heated to 65° C. for 10′ to destroy the polymerase activity and then cooled to 4° C.

Universal adaptors are ligated to the template DNA by addition of the following reagents: 2 μl (10 pmol) blunt end adaptor (FIG. 5A), 2 μl 3′ overhang adaptors and 5′ overhang adaptor (10 pmol each; FIG. 5A), and 1 μl T4 DNA Ligase (400 U, NEB), resulting in a final volume of 20 μl. The mixture was heated to 16° C. for 1 h and subsequently cooled to 4° C. Thirty microliters TE-Lo was added to each tube, resulting in a final concentration of 0.5 ng/μl

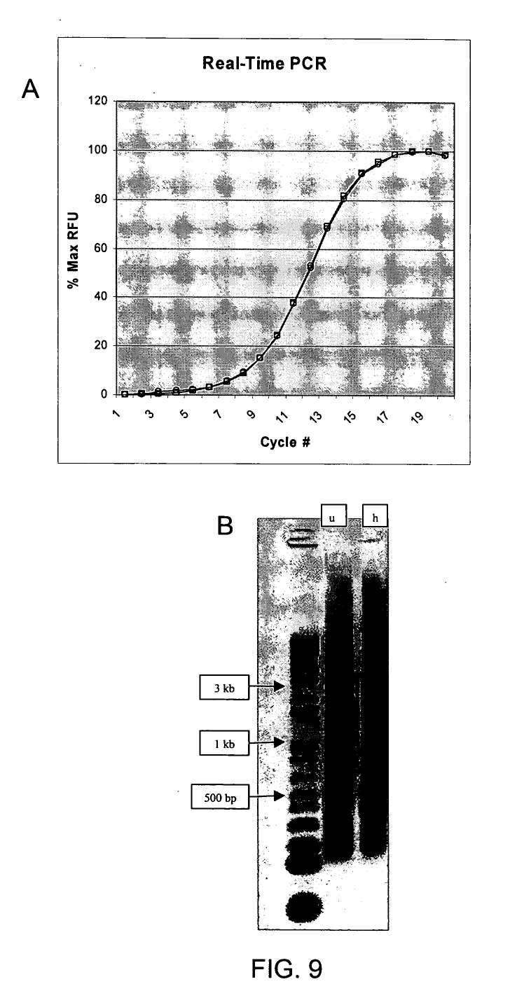

Extension of the 3′ end to fill in the universal adaptor and subsequent amplification of the library were carried out under the same conditions. Library (5 ng, 10 μl) was added to a 75 μl reaction containing 75 pmol T7 universal primer (SEQ ID NO: 11), 120 nmol dNTP, 1×PCR Buffer (Clontech), and 1×Titanium Taq (Clontech). Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 21 cycles of 94° C. 15″, 65° C. 2′. Real Time PCR measurement of the amplification and gel analysis of the amplified products following purification is depicted in FIG. 9.

The amplified products were purified using the Qiagen Qiaquick purification system and the amount of amplified material was determined spectrophotometrically (data not shown). Analysis of the amplified products using real-time PCR and a subset of the 103 human genomic STS markers indicates that 90% of the sites are within 2 fold of the average amplification. Furthermore, scatter plots of the individual markers indicates that they have a similar distribution to the products generated by mechanical fragmentation illustrated in FIG. 8.

### Example 3

### Whole genome amplification of human genomic DNA (10 ng Template) Fragmented by Chemical Methods

This example describes the amplification of 10 ng of genomic DNA that has been fragmented to an average size of 1 kb using chemical methods, specifically thermal fragmentation.

Human DNA (10 ng) was diluted in TE to a final volume of 10 μl. The DNA was subsequently heated to 95° C. for 4′, and then cooled to 4° C. Two microliters of 10×T4 DNA Ligase buffer was added to the DNA, and the mixture was heated to 95° C. for 10′, and then cooled to 4° C.

In order to generate competent ends for ligation, 40 nmol dNTP (Clontech), 0.1 pmol phosphorylated random hexamer primers (Genelink), and 5 Units Klenow (NEB) were added, and the resulting 15 μl reaction was incubated at 37° C. for 30′ and 12° C. for 1 h. Following incubation, the reaction was heated to 65° C. for 10′ to destroy the polymerase activity and then cooled to 4° C.

Universal adaptors were ligated to the template DNA by addition of the following reagents: 2 μl blunt end T7 adaptor (10 pmol), 2 μl T7 N overhang adaptors (10 pmol each), and 1 μl T4 DNA Ligase (400 U, NEB) resulting in a final volume of 20 μl. The mixture was heated to 16° C. for 1 h and subsequently cooled to 4° C.

Extension of the 3′ end to fill in the universal adaptor and subsequent amplification of the library were carried out under the same conditions. Library (5 ng) was added to a 75 μl reaction containing 75 pmol T7 universal primer (SEQ ID NO:11), 120 nmol dNTP, 1×PCR Buffer (Clontech), and 1×Titanium Taq (Clontech). Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 21 cycles of 94° C. 15″, 65° C. 2′.

The amplified products were purified using the Qiagen Qiaquick purification system and the amount of amplified material was determined spectrophotometrically. Analysis of the amplified products using real-time PCR and a subset of the 103 human genomic STS markers indicates that 90% of the sites are within 2 fold of the average amplification (data not shown). Furthermore, scatter plots of the individual markers indicates that they have a similar distribution to the products generated by mechanical fragmentation illustrated in FIG. 8.

### Example 4

### Utilization of a HEG-Linked Adaptor for Whole Genome Amplification of human genomic DNA (10 ng template) fragmented by Chemical Methods

This example describes the amplification of 10 ng of genomic DNA that has been fragmented to an average size of 1 kb using chemical methods, specifically thermal fragmentation.

Human DNA (10 ng) was diluted in TE to a final volume of 10 μl. DNA was subsequently heated to 95° C. for 4′, and then cooled to 4° C. Two microliters of 10×T4 DNA Ligase buffer was added to the DNA, and the mixture was heated to 95° C. for 10′, and then cooled to 4° C.

In order to generate competent ends for ligation, 40 nmol dNTP (Clontech), 0.1 pmol phosphorylated random hexamer primers (Genelink), and 5 Units Klenow (NEB) were added, and the resulting 15 μl reaction was incubated at 37° C. for 30′, and 12° C. for 1 h. Following incubation, the reaction was heated to 65° C. for 10′ to destroy the polymerase activity and then cooled to 4° C.

T7HEG adaptors were ligated to the template DNA by addition of the following reagents: 2 μl T7HEG adaptor (10 pmol; SEQ ID NO:36; FIG. 5B), 2 μl H₂O, and 1 pt T4 DNA Ligase (400 U, NEB) resulting in a final volume of 20 μl. The mixture was heated to 16° C. for 1 h and subsequently cooled to 4° C.

Extension of the 3′ end to fill in the universal adaptor and subsequent amplification of the library were carried out under the same conditions. Library (5 ng) was added to a 75 μl reaction containing 75 pmol T7 universal primer (SEQ ID NO: 11), 120 nmol dNTP, 1×PCR Buffer (Clontech), and 1×Titanium Taq (Clontech). Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 21 cycles of 94° C. 15″, 65° C. 2′.

The amplified products were purified using the Qiagen Qiaquick purification system and the amount of amplified material was determined spectrophotometrically. Gel analysis (FIG. 9B) indicates that the size of the amplified products generated with the T7HEG adaptor (h) is identical to those generated with the universal adaptor (u). Analysis of the amplified products using real-time PCR and a subset of the 103 human genomic STS markers indicates that 90% of the sites are within 2 fold of the average amplification (data not shown). Furthermore, scatter plots of the individual markers indicates that they have a similar distribution to the products generated by mechanical fragmentation illustrated in FIG. 8.

### Example 5

### Utilization of a Heg Linked Adaptor Where the Second Polishing Step is Combined with Ligation for Whole Genome Amplification of human genomic DNA (10 ng template) fragmented By Chemical Methods

This example describes the amplification of 10 ng of genomic DNA that has been fragmented to an average size of 1 kb using chemical methods, specifically thermal fragmentation.

Human DNA (10 ng) was diluted in TE to a final volume of 10 μl. DNA was subsequently heated to 95° C. for 4′, and then cooled to 4° C. Two microliters of 10×T4 DNA Ligase buffer was added to the DNA and the mixture was heated to 95° C. for 10′, and then cooled to 4° C.

In order to generate competent ends for ligation, 40 nmol dNTP (Clontech), 1 pmol phosphorylated random hexamer primers (Genelink), and 5 Units Klenow (NEB) were added and the resulting 15 μl reaction was incubated at 37° C. for 30′.

The completion of the polishing reaction was combined with the ligation reaction as follows. T7HEG adaptors were ligated to the template DNA by addition of the following reagents: 2 μl T7HEG (10 pmol; SEQ ID NO:36), 2 μl H₂O, and 1 μl T4 DNA Ligase (400 U, NEB) resulting in a final volume of 201l. The mixture was heated to 16° C. for 1 h and subsequently cooled to 4° C.

Extension of the 3′ end to fill in the universal adaptor and subsequent amplification of the library were carried out under the same conditions. Library (5 ng) was added to a 75 μl reaction containing 75 pmol T7 universal primer (SEQ ID NO:11), 120 nmol dNTP, 1×PCR Buffer (Clontech), 1×Titanium Taq (Clontech). Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 21 cycles of 94° C. 15″, 65° C. 2′.

The amplified products were purified using the Qiagen Qiaquick purification system and the amount of amplified material was determined spectrophotometrically. Analysis of the amplified products using real-time PCR and a subset of the 103 human genomic STS markers indicates that 90% of the sites are within 2 fold of the average amplification (data not shown). Furthermore, scatter plots of the individual markers indicates that they have a similar distribution to the products generated by mechanical fragmentation illustrated in FIG. 8.

### Example 6

### Utilization of a Heg Linked Adaptor in a Single Polishing Ligation Step for Whole Genome Amplification of Human genomic DNA (10 ng template) fragmented by chemical Methods

This example describes the amplification of 10 ng of genomic DNA that has been fragmented to an average size of 1 kb using chemical methods, specifically thermal fragmentation.

Human DNA (10 ng) was diluted in TE to a final volume of 10 μl. DNA was subsequently heated to 95° C. for 4′, and then cooled to 4° C. Two microliters of 110×T4 DNA Ligase buffer was added to the DNA, and the mixture was heated to 95° C. for 10′, and then cooled to 4° C.

In order to generate competent ends for ligation and ligate adaptors to these ends, 40 nmol dNTP (Clontech), 1 pmol phosphorylated random hexamer primers (Genelink), 5 U Klenow (NEB), 2 μl T7HEG adaptor (10 pmol; SEQ ID NO:36; FIG. 5B), 2 μl H₂O, and 1 μl T4 DNA Ligase (400 U, NEB) resulting in a final volume of 20 μl were mixed together and incubated at 37° C. for 90′.

Extension of the 3′ end to fill in the universal adaptor and subsequent amplification of the library were carried out under the same conditions. Library (5 ng) was added to a 75 μl reaction containing 75 pmol T7 universal primer (SEQ ID NO: 11), 120 nmol dNTP, 1×PCR Buffer (Clontech), and 1×Titanium Taq (Clontech). Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 21 cycles of 94° C. 15″, 65° C. 2′.

The amplified products were purified using the Qiagen Qiaquick purification system and the amount of amplified material was determined spectrophotometrically. Analysis of the amplified products using real-time PCR and a subset of the 103 human genomic STS markers indicates that 90% of the sites are within 2 fold of the average amplification (data not shown). Furthermore, scatter plots of the individual markers indicates that they have a similar distribution to the products generated by mechanical fragmentation illustrated in FIG. 8.

### Example 7

### Converting DNA Into Library by Simultaneous DNASE I Cleavage and Linker Ligation for PCR Amplification

A. Development of Buffer System

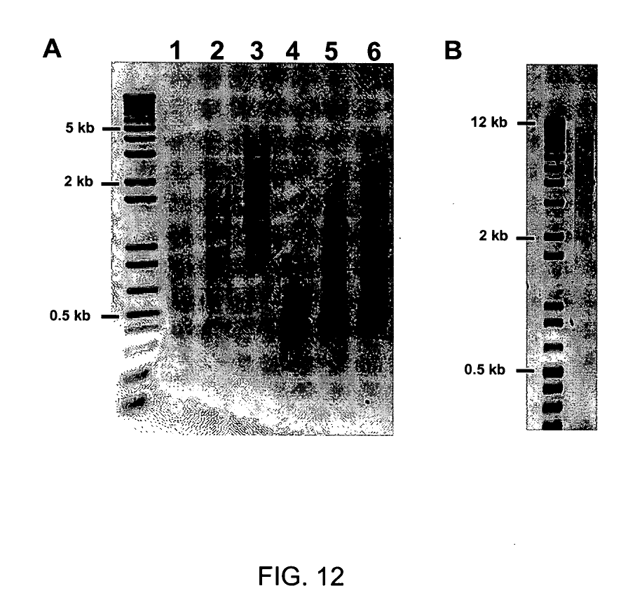

In order to achieve simultaneous DNAse I cleavage and ligation, a buffer compatible with both enzymatic reactions was developed. DNase I requires Mn²⁺ ions in order to randomly cleave both strands of double-stranded DNA at approximately the same site. T4 DNA ligase requires ATP and Mg²⁺ or Mn²⁺ ions for catalytic activity, and the ligation reaction buffer typically also contains DTT. Based upon the above conditions, two buffers were formulated. The first, termed Buffer M10, comprises 50 mM Tris-Cl (pH 7.5), 10 mM MnCl₂, 0.1 mM CaCl₂, 10 mM DTT, 1 mM ATP, and 25 μg/mL BSA. The 10 mM MnCl₂ concentration was chosen for this buffer, based upon the DNase I manufacturer's recommended conditions for efficient cleavage. The second buffer, termed M3, comprises 50 mM Tris-Cl (pH 7.5), 3 mM MnCl₂, 10 mM DTT, and 1 mM ATP. The 3 mM MnCl₂ concentration was chosen for this buffer, based upon the optimal concentration for T4 DNA ligase. DNase I cleavage was determined to function in both buffers, but proceeded much more rapidly in Buffer M10 than in Buffer M3 (FIG. 12).

B. Design and Synthesis of Linker Cocktail

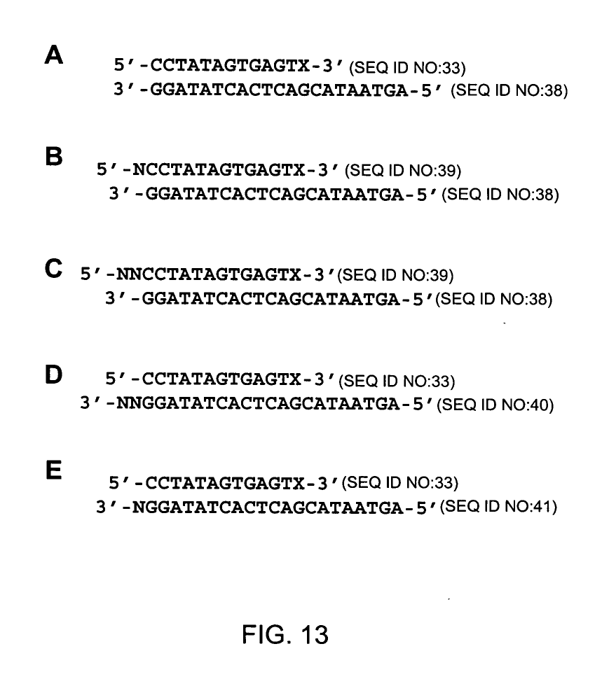

Since fragments of DNA cleaved by DNase I are blunt-ended or have protruding termini of only one or two nucleotides in length, appropriate linkers (FIG. 13) were designed that could be ligated to each type of fragment end. FIG. 13A illustrates a linker designed for ligation to a blunt ended genomic DNA fragment, while FIGS. 13B-13E illustrate linkers designed for ligation to genomic DNA fragment ends with one or two nucleotide overhangs. To synthesize each type of linker, 1 nmole of the longer oligonucleotide and 2 nmole of the shorter oligonucleotide were incubated in 100 μL of 10 mM KCl for 1 minute at 65° C. and then allowed to cool slowly to room temperature.

C. Library Construction

For construction of libraries in Buffer M10, 10 ng/μL human genomic DNA, 1-6×10⁻⁵ Units/μL of DNase I (Fermentas), 200 units/μL of T4 DNA ligase (New England Biolabs), and 2 pmoles/μL of each type of linker were incubated in Buffer M10 at 16° C. between 1 hour and 21 hours. The reaction was stopped at the appropriate time by adding 1 μL EGTA, pH 8.0, per 10 μL reaction mix and heating for 10 minutes at 65° C.

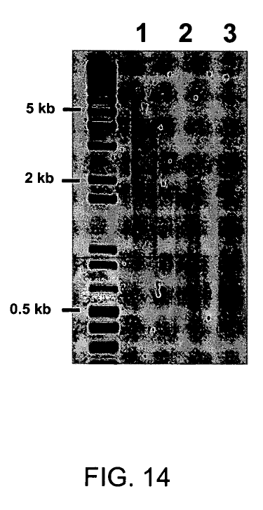

For construction of libraries in Buffer M3, 10 ng/μL human genomic DNA, 1-3×10⁻⁵ Units/μL of DNase I (Fermentas), 100 units/μL of T4 DNA ligase (New England Biolabs), and 1 pmole/μL of each type of linker were incubated in Buffer M3 at 16° C. for 18-21 hours. The reaction was stopped by heating for 10 minutes at 75° C. Under these conditions, the size of the linkered DNA fragments ranged from 0.5 kb to 5 kb based on Ethidium Bromide staining of 80 ng of library electrophoresed on a 1.0% agarose gel (FIG. 14). Titration of the amount of DNase I resulted in the average fragment size varying between 3 kb (lane 1) and 0.7 kb (lane 3).

D. Amplification of Fragments

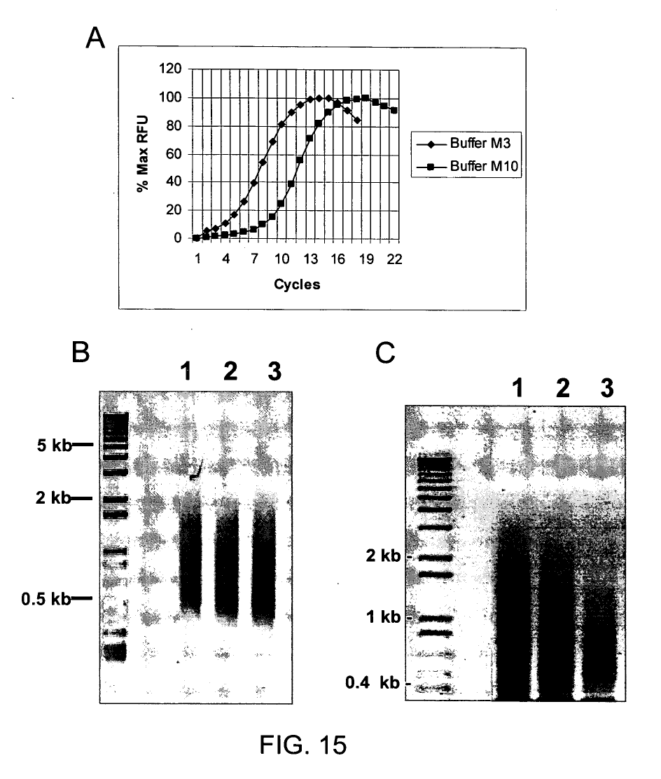

As described in FIGS. 11 and 13, only one oligonucleotide of each linker was ligated to the genomic DNA fragment ends. To create a sequence fully complementary to the longer oligonucleotide and covalently attached to the duplex DNA fragment, five ng of the library constructed in M10 Buffer was incubated at 75° C. for 15 minutes in 75 μL of PCR buffer (40 mM Tricine-KOH (pH 8.0), 16 mM KCl, 3.5 mM MgCl₂, 3.75 μg/mL BSA) comprising 200 uM each of dATP, dCTP, dGTP, and dTTP, 1 uM of a primer having the sequence 5′-GTAATACGACTCACTATA-3′ (SEQ ID NO:11), and 0.75 μL of Titanium Taq Polymerase (Clontech). For library constructed in M3 Buffer, 10 ng of the library was was incubated at 75° C. for 15 minutes in 25 μL of PCR buffer (40 mM Tricine-KOH (pH 8.0), 16 mM KCl, 7.0 mM MgCl₂, 3.75 μg/mL BSA) containing 400 μM each of dATP, dCTP, dGTP, and dTTP, 2 uM of a primer having the sequence 5′-GTAATACGACTCACTATA-3′ (SEQ ID NO: 11), and 0.25 μL of Titanium Taq Polymerase. The reaction mixture was then heated to 95° C. for 2 minutes for denaturation and the linkered fragments replicated by incubating at 94° C. for 15 seconds to allow denaturation followed by incubating at 65° C. for 2 minutes to allow primer annealing and extension. The replication steps were repeated 22 times for libraries constructed in Buffer M10 and 18 times for libraries constructed in Buffer M3, in order to generate 5-8 μg of amplified DNA. By analyzing the PCR amplification kinetics in real-time (FIG. 15A), it was determined that libraries constructed in Buffer M3 are more efficiently end-linkered than libraries constructed in Buffer M10. Thus, in the best mode, buffers favoring ligation over cleavage (M3) are used rather than buffers favoring cleavage over ligation (M10). When amplified products from libraries constructed in Buffer M3 were analyzed by real-time PCR using 24 human genomic STS markers, 90% of the 24 sites are within 2 fold of the average amplification (data not shown).

Ethidium bromide staining of amplified DNA electrophoresed on a 1.0% agarose gel indicates that fragments between 0.2 kb and 5 kb were amplified (FIGS. 15B and 15C). The size distribution of fragments obtained before (FIG. 14, lanes 1-3) and after amplification (FIG. 15B, lanes 1-3) was conserved, demonstrating that the majority of the fragments were amplified efficiently. The ability to generate libraries of different average fragment size (FIG. 15C) from the same digestion/ligation reaction was demonstrated by removing aliquots at different time points.

### Example 8

### Incorporation of Individual Identification DNA Tags by Whole Genome Amplification; Recovery of the Individual WGA Libraries From a Mixture of Several WGA Libraries

This example describes two processes of tagging an individual WGA library with a DNA identification sequence (ID) for the purpose of subsequent recovery of this library from a mixture containing WGA libraries labeled with different tags. This situation can occur unintentionally when manipulating or storing very large numbers of WGA DNA samples or intentionally when there is a need to prevent an unauthorized access to genetic information within the stored libraries.

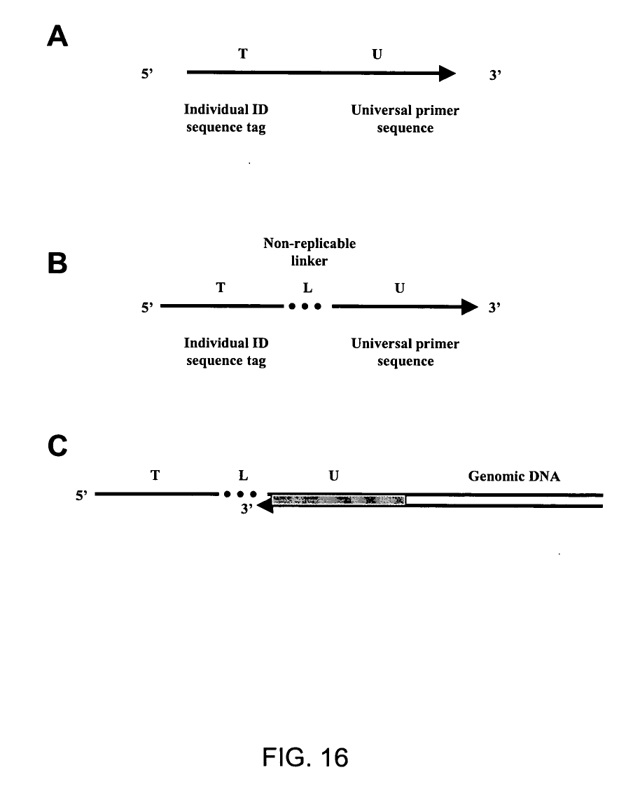

Both processes involve universal primers with universal sequence U at the 3, end and an individual ID sequence tag at the 5′ end (FIG. 16). In the first case, the universal primer is comprised of regular bases (A, T, G and C) and can be replicated (FIG. 16A). In the second case, the universal primer has a non-nucleotide linker L (for example, hexa ethylene glycol, HEG) and can't be replicated (FIGS. 16B and 16C).

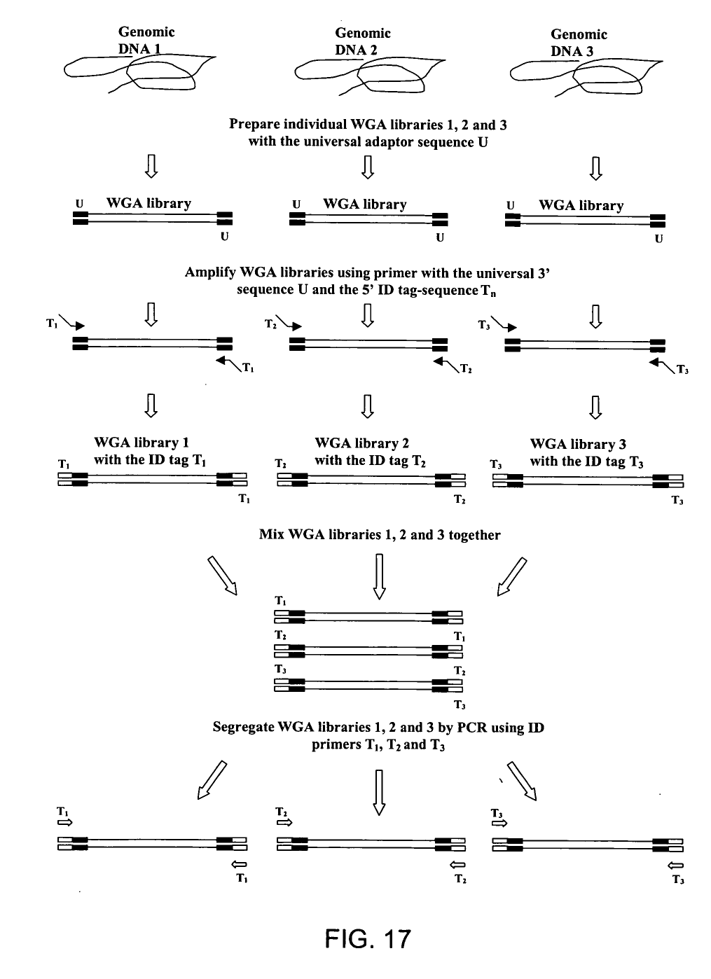

The process of tagging, mixing and recovery of 3 different WGA libraries using replicable universal primers is shown in FIG. 17. It comprises at least four steps:

1) Three genomic DNA samples are converted into 3 WGA libraries using the methods described earlier in the patent application;

2) Three WGA libraries are amplified using 3 individual replicable universal primers T₁U, T₂U, and T₃U with the corresponding ID DNA tags T₁, T₂, and T₃ at the 5′ end (FIG. 16A);

3) All three libraries are mixed together. Any attempt to amplify and genotype the mix would result in a mixed pattern; and

4) The WGA libraries are segregated by PCR using individual ID primers tags T₁, T₂, and T₃.

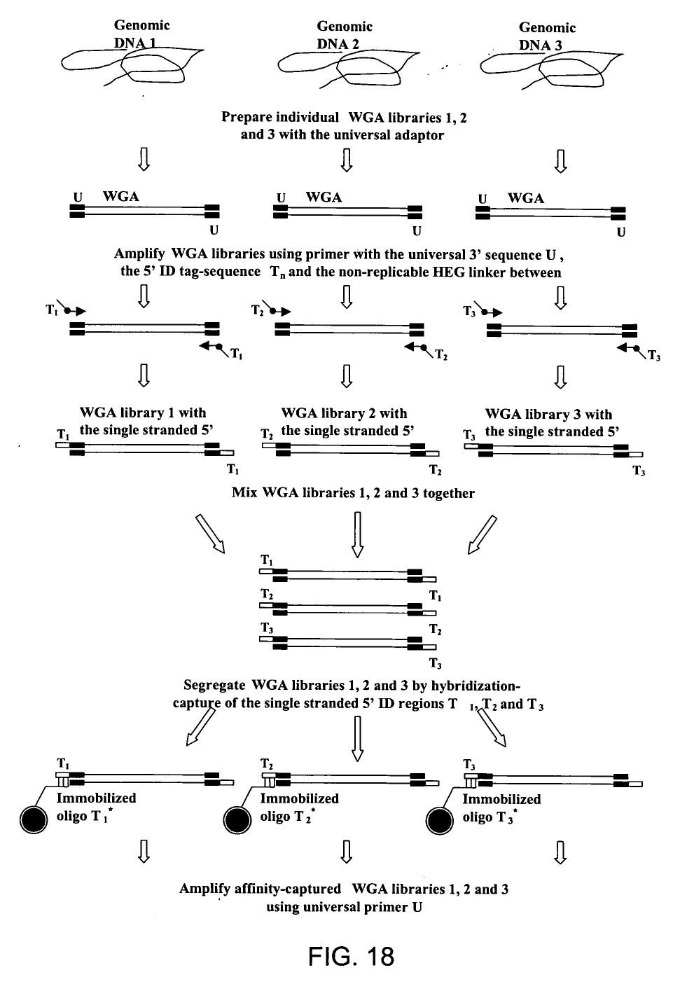

The process of tagging, mixing and recovery of 3 different WGA libraries using non-replicable universal primers is shown in FIG. 18. It comprises at least five steps:

1) Three genomic DNA samples are converted into 3 WGA libraries using the method described elsewhere herein;

2) Three WGA libraries are amplified using 3 individual non-replicable universal primers T₁U, T₂U, and T₃U with the corresponding ID DNA tags T₁, T₂, and T₃ at the 5′ end (FIGS. 16B and 16C). The resulting products have 5′ single stranded tails formed by ID regions of the primers;

3) All three libraries are mixed together. Any attempt to amplify and genotype the mix would result in a mixed pattern;

4) The WGA libraries are segregated by hybridization of their 5′ tails to the complementary oligonucleotides T₁*, T₂*, and T₃* immobilized on the solid support; and

5) The segregated libraries are amplified by PCR using universal primer U.

### Example 9

### WGA Libraries in the Micro-Array Format

For archiving purposes, individual WGA libraries can be immobilized on a micro-array. The micro-array format would allow storage of tens or even hundred thousand immortalized DNA samples on one small microchip while allowing rapid, automated access- to them.

There are two ways to immobilize WGA libraries to a micro-array: covalently and non-covalently.

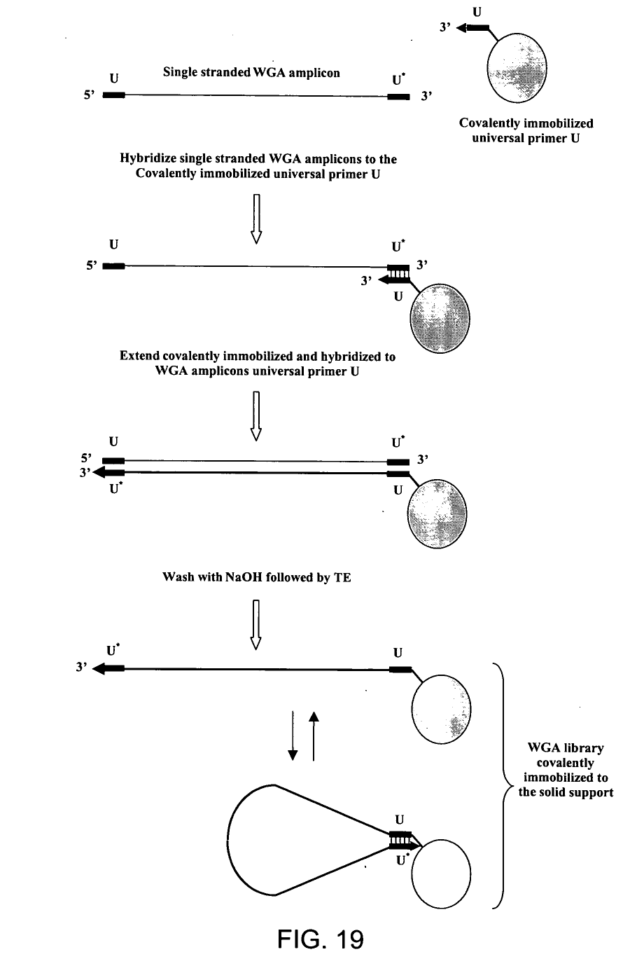

FIG. 19 shows the process of covalent immobilization. It comprises 3 steps:

Step 1. Hybridization of single stranded (denatured) WGA amplicons to the universal primer-oligonucleotide U covalently attached to the solid support.

Step 2. Extension of the primer U and replication of the hybridized amplicons by DNA polymerase.

Step 3. Washing with 100 mM sodium hydroxide solution and TE buffer.

Non-covalent immobilization can be achieved by using WGA libraries with affinity (i.e. biotin) or identification DNA tags at the 5′ ends of amplicons. Biotin can be located at the 5′ end of the universal primer U. Single stranded 5′ affinity or/and ID tags can be introduced by using non-replicable primers (FIGS. 16B and 16C; FIG. 18). Biotinylated libraries can be immobilized through the streptavidin covalently attached to the surface of the micro-array. WGA libraries with the 5′ overhangs can be hybridized to the oligonucleotides covalently attached to the surface of the micro-array.

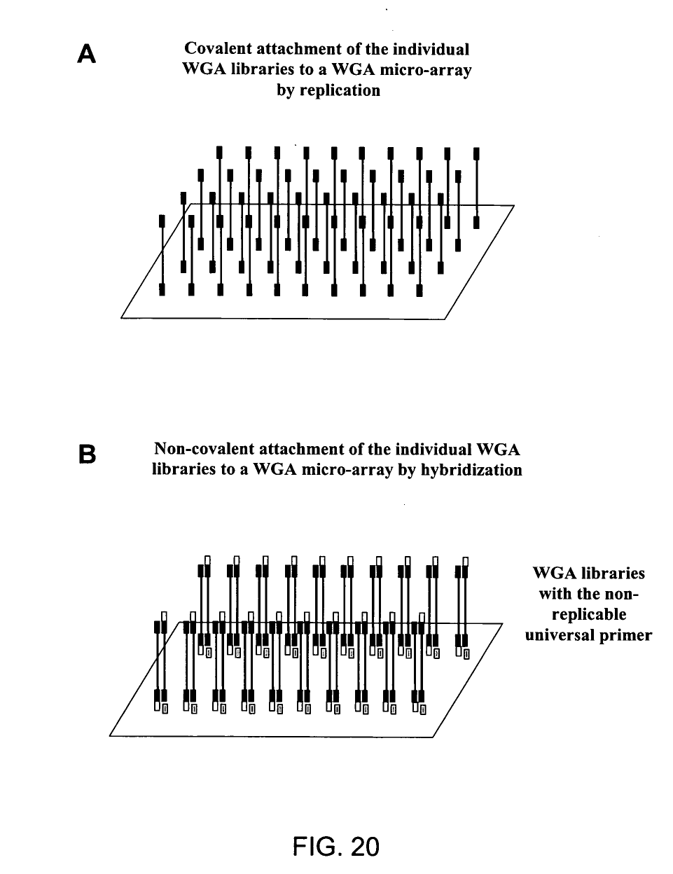

Both covalently and non-covalently arrayed libraries are shown in FIG. 20.

### Example 10

### Repeated Usage of Immobilized WGA Libraries

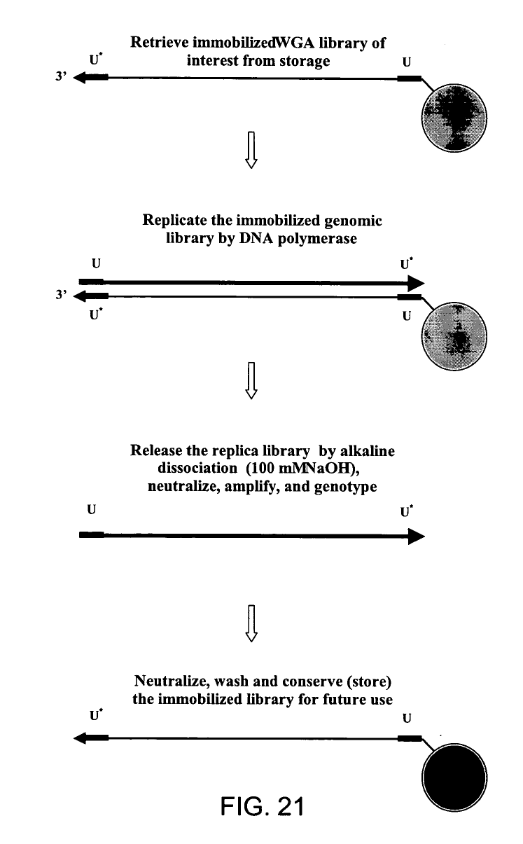

Covalently immobilized WGA libraries (or libraries immobilized through the biotin-streptavidin interaction) can be used repeatedly to produce replica libraries for whole genome amplification (FIG. 21). In this case, the process comprises at least four steps:

1) Retrieval of the immobilized library from the long term storage;

2) Replication of the immobilized library using DNA polymerase and universal primer U;

3) Dissociating replica molecules by sodium hydroxide, neutralization and amplification; and

4) Neutralization and return of the solid phase library for long term storage.

### Example 11

### Purification of the WGA Products Using a Non-Replicable Primer Affinity Tag and DNA Immobilization by Hybridization

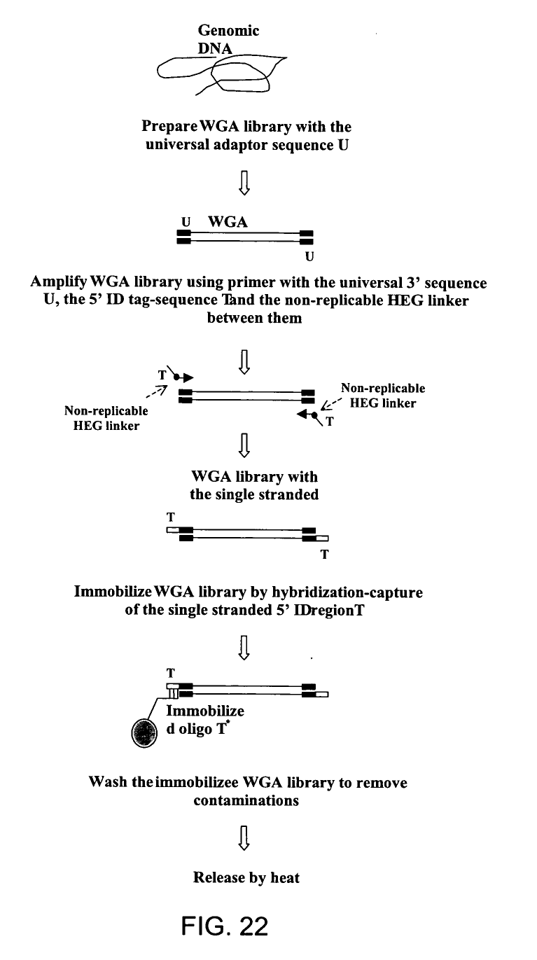

For many applications, purity of the amplified DNA is critical. WGA libraries with the 5′ overhangs can be hybridized to the oligonucleotides covalently attached to the surface of magnetic beads, tube or micro-plate, washed with TE buffer or water to remove excess of dNTPs, buffer and DNA polymerase and then released by heating in a small volume of TE buffer. For this purpose, the single stranded 5′ affinity tag can be introduced by using a non-replicable primer (FIGS. 16B and 16C; and FIG. 22).

### Example 12

### Library Creation and Whole Genome Amplification of DNA Isolated from Serum

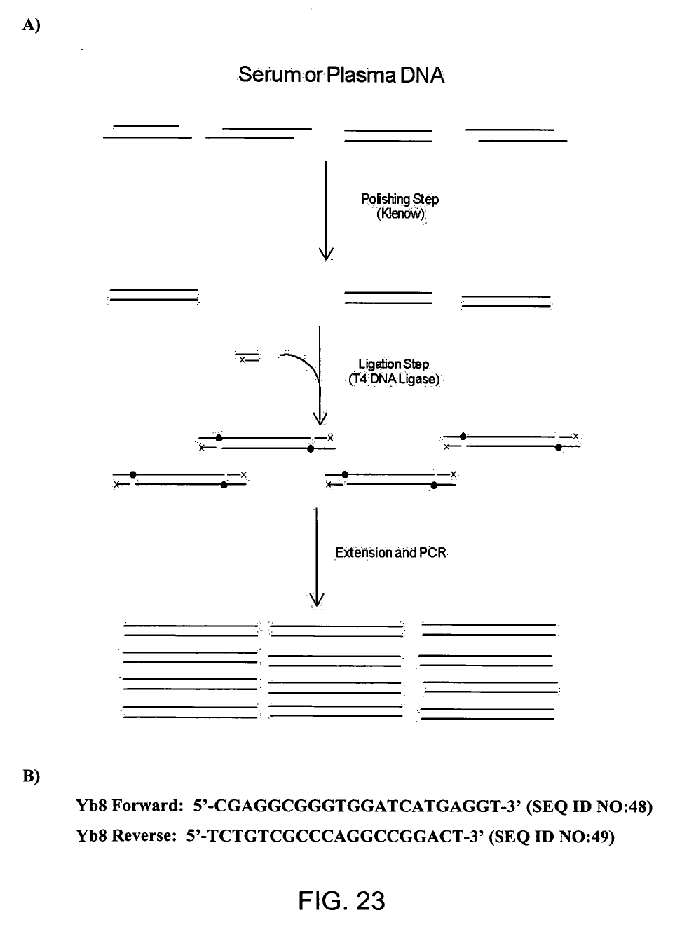

This example, illustrated in FIG. 23A, describes the amplification of genomic DNA that has been isolated from serum or plasma. Blood was collected into 8 ml vacutainer no-additive tubes (serum) or EDTA tubes (plasma). The serum tubes (no additive) were allowed to sit at room temperature for 2 h and at 4° C. overnight. The tubes were centrifuged for 10′ at 1,000×G with minimal acceleration and braking. The serum was subsequently transferred to a clean tube. The plasma tubes (EDTA) were incubated at 4° C. for 1 hr and centrifuged for 10′ at 1,000×G with minimal acceleration and braking. The plasma was subsequently transferred to a clean tube. Isolated serum and plasma samples may be used immediately for DNA extraction or stored at −20° C. prior to use.

DNA from 1 ml of serum or plasma was purified using the DRI ChargeSwitch Blood Isolation kit according to the manufacturer's protocols. The resulting DNA was precipitated using the pellet paint DNA precipitation kit (Novagen) according to the manufacturer's instructions and the sample was resuspended in TE-Lo to a final volume of 30 μl for serum and 10 μl for plasma. The quantity and concentration of DNA present in the sample was quantified by real-time PCR using Yb8 Alu primer pairs (FIG. 23B; SEQ ID NO:48 and 49). Briefly, 25 μl reactions consisting of 1×PCR Buffer, 400 uM dNTP, 0.5×Titanium Taq, 200 nM each of Yb8 Forward (SEQ ID NO: 48) and Yb8 Reverse (SEQ ID NO: 49) primers, and 1:100,000 dilutions of fluorescein calibration dye and SYBR Green I were amplified for 40 cycles at 94° C. for 15 sec and 74° C. for 1 min. Standards corresponding to 10, 1, 0.1, 0.01, and 0.001 ng of genomic DNA were used and the serum DNA quantities and concentrations were calculated by standard curve fit (I-Cycler software, Bio-Rad).

The first step of this embodiment of library preparation is to produce blunt ends on all DNA molecules. This step comprises incubation with at least one polymerase. Specifically, 2 μl of a mix containing 1.1 μl 10×T4 DNA ligase buffer, 200 nmol dNTP (Clontech), 0.2 U Klenow (USB) and H₂O were added to 10 μl of isolated serum (3 ng) or plasma DNA (3 ng) in TE-Lo. The reaction was carried out at 25° C. for 15′, and the polymerase was inactivated by heating the mixture at 75° C. for 15′, and then cooling to 4° C. Universal adaptors were ligated to the 5′ ends of the DNA using T4 DNA ligase by addition of 2 μl blunt end adaptor (10 pmol, FIG. 5A) and 1 μl T4 DNA Ligase (2,000 U). The reaction was carried out for 1 h at 16° C., 10′ at 75° C., and then held at 4° C. until use. Alternatively, the libraries can be stored at −20° C. for extended periods prior to use.

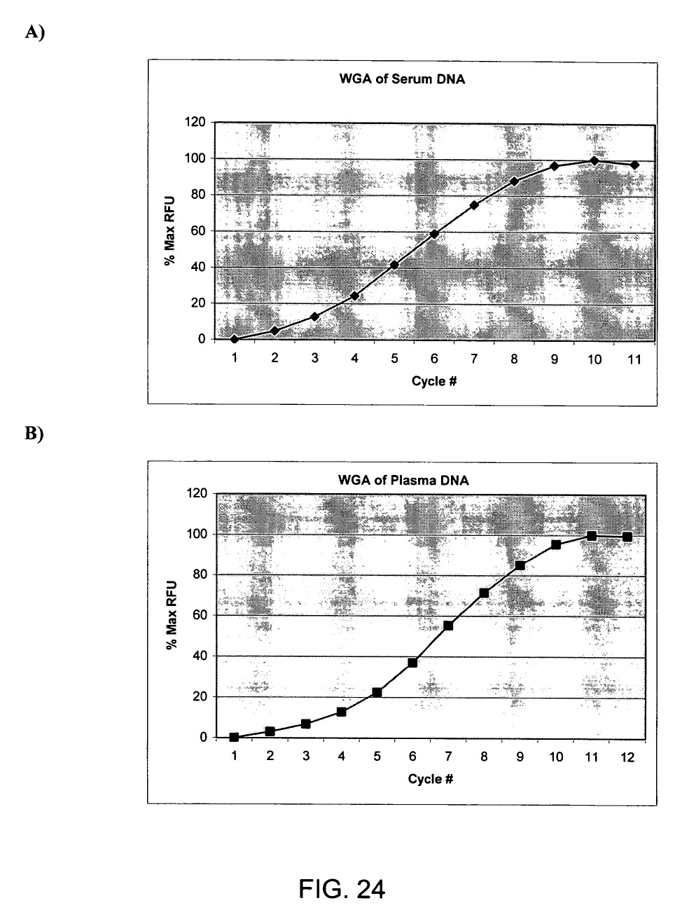

Extension of the 3′ end to fill in the universal adaptor and subsequent amplification of the library were carried out under the same conditions. Three ng of library is added to a 75 μl reaction comprising 75 pmol T7 universal primer (SEQ ID NO: 11), 200 nmol dNTP, 1×PCR Buffer (Clontech), 1×Titanium Taq. Fluorescein calibration dye (1:100,000) and SYBR Green I (1:100,000) are also added to allow monitoring of the reaction using the I-Cycler Real-Time Detection System (Bio-Rad). The samples are initially heated to 75° C. for 15′ to allow extension of the 3′ end of the fragments to fill in the universal adaptor sequence and displace the short, blocked fragment of the universal adaptor. Subsequently, amplification is carried out by heating the samples to 95° C. for 3′30″, followed by 11-14 cycles of 94° C. 15″, 65° C. 2′. The cycle number is dependent on the amount of template in the reaction. Typically, for 3 ng of library the optimal number of cycles is 12 for serum (FIG. 24A) and 13 for plasma (FIG. 24B).

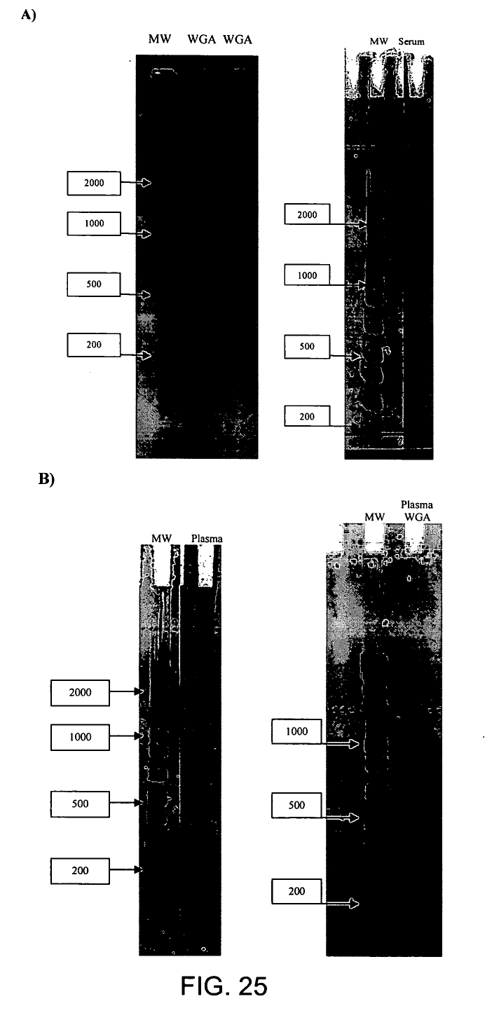

The amplified material was purified by Millipore Multiscreen PCR plates and quantified spectrophotometrically. Gel analysis of the amplified products indicated a size distribution (200 bp to 1 kb) similar to the original serum DNA for both serum (FIG. 25A) and plasma (FIG. 25B). Additionally, the amplified DNA was analyzed using real-time, quantitative PCR using a panel of human genomic STS markers. The markers that make up the panel are listed in Table IV. Quantitative Real-Time PCR was performed using an I-Cycler Real-Time Detection System (Bio-Rad), as per the manufacturer's directions. Briefly, 25 μl reactions consisting of 1×PCR Buffer, 400 uM dNTP, 0.5×Titanium Taq, 200 nM primers, and 1:100,000 dilutions of fluorescein calibration dye and SYBR Green I were amplified for 40 cycles at 94° C. for 15 sec and 65° C. for 1 min. Standards corresponding to 10, 1, and 0.2 ng of fragmented DNA were used for each STS, quantities were calculated by standard curve fit for each STS (I-Cycler software, Bio-Rad) and were plotted as distributions.

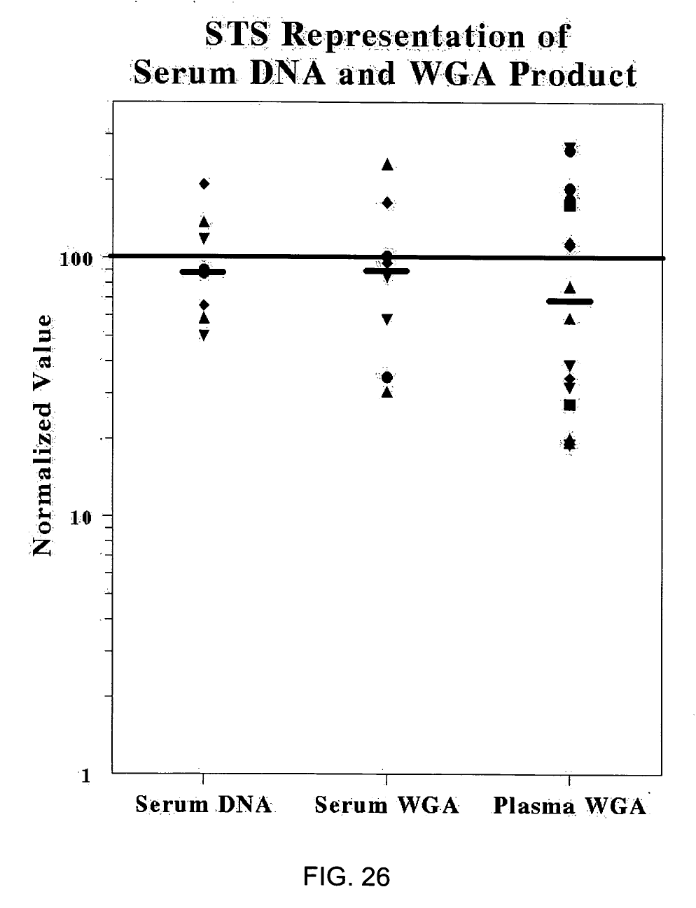

Quantitative real-time PCR of the WGA products from serum demonstrated that all of the 8 markers were within a factor of 4 of the mean amplification. In comparison, analysis of the serum DNA indicated that the same 8 markers were within a factor of 2 of the mean amplification. These results indicate that the representation of the original serum DNA is maintained following WGA. Quantitative real-time PCR of the WGA products from plasma demonstrated that all of the 8 markers were within a factor of 5 of the mean amplification. FIG. 26 is a scatterplot of the representation of the human genomic STS markers in the serum DNA and the amplified DNA from both serum and plasma.

### Example 13

### Library Creation and Whole Genome Amplification of DNA Isolated From Serum Using Overhanging Adaptors Specific For the ends of dna present in serum and plasma

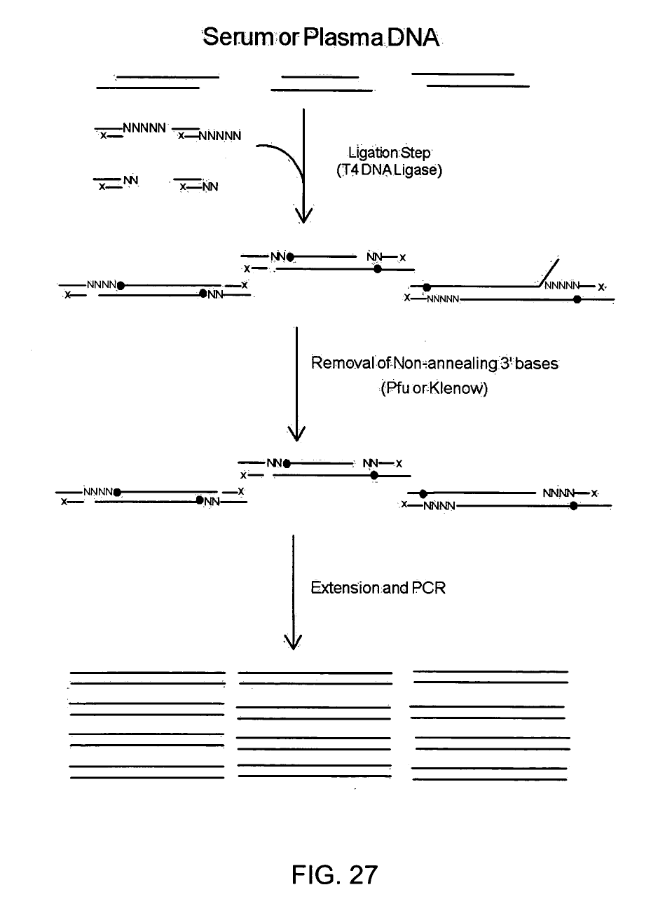

This example, illustrated in FIG. 27, describes the amplification of genomic DNA that has been isolated from serum. Blood was collected into 8 ml vacutainer no-additive tubes (serum) or EDTA tubes (plasma). The serum tubes (no additive) were allowed to sit at room temperature for 2 h and at 4C overnight. The tubes were centrifuged for 10′ at 1,000×G with minimal acceleration and braking. The serum was subsequently transferred to a clean tube. The plasma tubes (EDTA) were incubated at 4° C. for 1 hr and centrifuged for 10′ at 1,000×G with minimal acceleration and braking. The plasma was subsequently transferred to a clean tube. Isolated serum and plasma samples may be used immediately for DNA extraction or stored at −20° C. prior to use.

DNA from 1 ml of serum or plasma was purified using the DRI ChargeSwitch Blood Isolation kit according to the manufacturer's protocols. The resulting DNA was precipitated using the pellet paint DNA precipitation kit (Novagen) according to the manufacturer's instructions and the sample was resuspended in 30 μl (serum) or 10 μl (plasma) TE-Lo. The quantity and concentration of DNA present in the sample was quantified by real-time PCR using Yb8 Alu primer pairs (FIG. 23B; SEQ ID NO:48 and SEQ ID NO: 49). Briefly, 25 μl reactions consisting of 1×PCR Buffer, 400 uM dNTP, 0.5×Titanium Taq, 200 nM each of Yb8 Forward (SEQ ID NO: 48) and Yb8 Reverse (SEQ ID NO: 49) primers, and 1:100,000 dilutions of fluorescein calibration dye and SYBR Green I were amplified for 40 cycles at 94° C. for 15 sec and 74° C. for 1 min. Standards corresponding to 10, 1, 0.1, 0.01, and 0.001 ng of genomic DNA were used and the serum and plasma DNA quantities and concentrations were calculated by standard curve fit (1-cycler software, Bio-Rad).

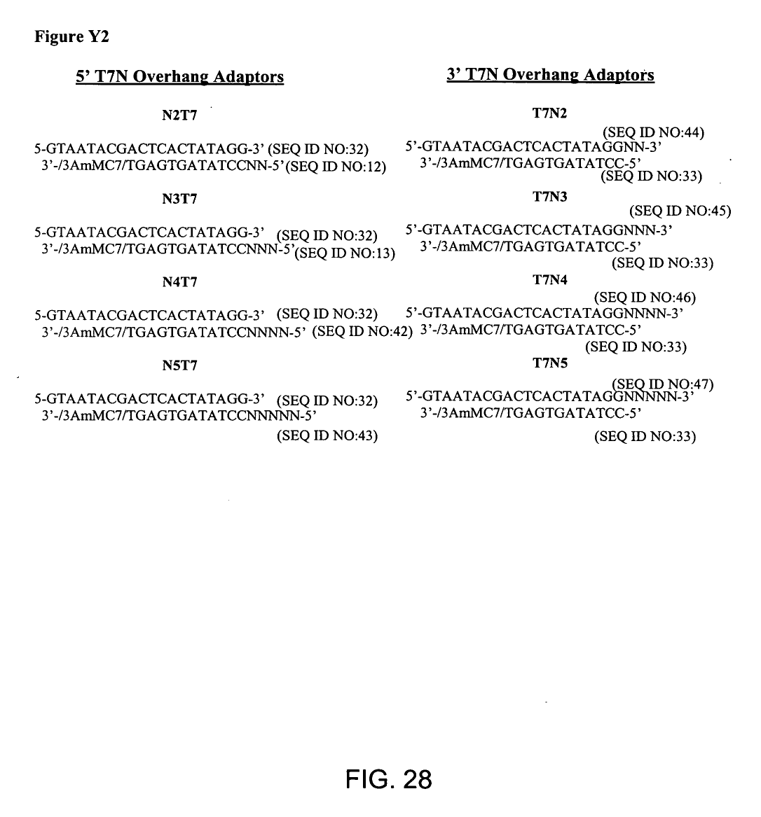

Universal adaptors were ligated to the 5′ ends of the serum DNA (3 ng) or plasma DNA (1 ng) using T4 DNA ligase by addition of 2 μl of each adaptor mix, 1.7 μl 10×T4 DNA Ligase Buffer, 0.3 μl H₂O, and 1 μl T4 DNA Ligase (2,000 U). The reaction was carried out for 1 h at 16° C., 10′ at 75° C., and then held at 4° C. until use. Alternatively, the libraries can be stored at −20° C. for extended periods prior to use. The adaptor mix consists of a combination of specific adaptors that most effectively anneal and ligate to the serum and plasma DNA template. The adaptors are illustrated in FIG. 28 and consist of 10 pmol each of N5T7, N2T7, T7N2, and T7N5. The 3′ T7N overhang adaptors are created by mixing 10 pmol of each of the long oligos containing either 2 bp or 5 bp 3′ N bases with 40 pmol of the short, 3′AmMC7 oligo in the presence of 10 mM KCl, incubating at 65° C. for 1′, slowly cooling to room temperature, and then placing them on ice. The assembled adaptors are stored at −20° C. until use. The 5′ T7N overhang adaptors consist of a mixture of 20 pmol of the long oligo with 20 pmol of each of the 3′ AmMC7 oligo containing either 2 bp or 5 bp 5′N bases and are annealed using the same procedure as for the 3′ T7N overhang adaptors.

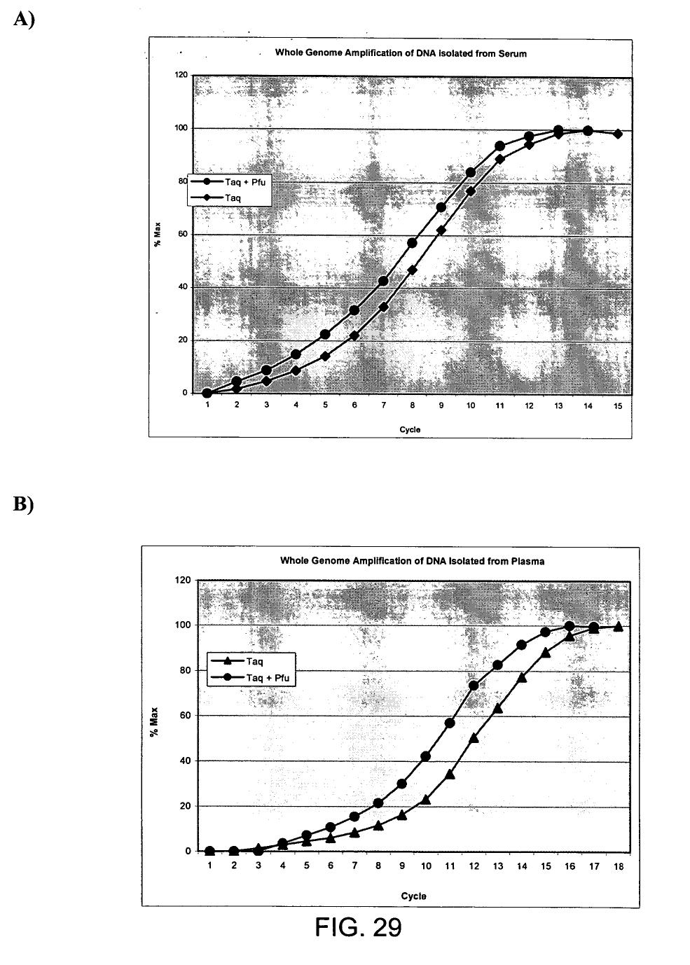

Extension of the 3′ end to fill in the universal adaptor and subsequent amplification of the library were carried out under the same conditions. Three nanograms (serum) or 5 ng (plasma) of library is added to a 75 μl reaction comprising 75 pmol T7 universal primer (SEQ ID NO:11), 120 nmol dNTP, 1×PCR Buffer (Clontech), 1×Titanium Taq, in the presence or absence of 0.25 U pfu (Stratagene). Fluorescein calibration dye (1:100,000) and SYBR Green I (1:100,000) are also added to allow monitoring of the reaction using the I-Cycler Real-Time Detection System (Bio-Rad). The samples are initially heated to 75° C. for 15′ to allow extension of the 3′ end of the fragments to fill in the universal adaptor sequence and displace the short, blocked fragment of the universal adaptor. The addition of Pfu results in removal of any 3′ non-complementary bases from the plasma or serum DNA (See FIG. 27) to improve the efficiency of the extension reaction. Subsequently, amplification is carried out by heating the samples to 95° C. for 3′30″, followed by 11-14 cycles of 94° C. 15″, 65° C. 2′. The cycle number is dependent on the amount of template in the reaction. Typically, for 3 ng of library the optimal number of cycles is 13 (FIG. 29A).

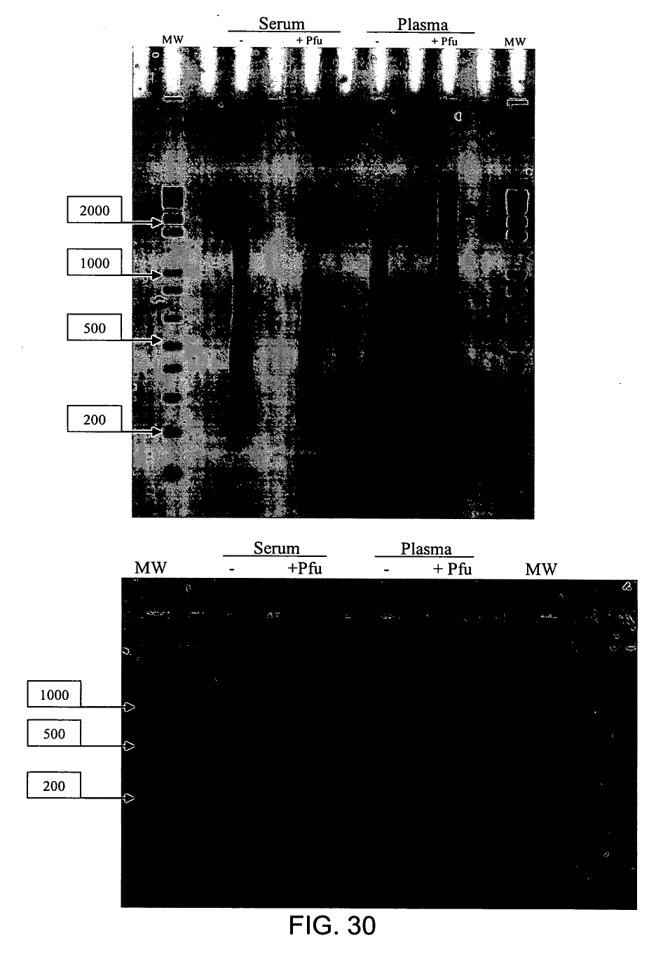

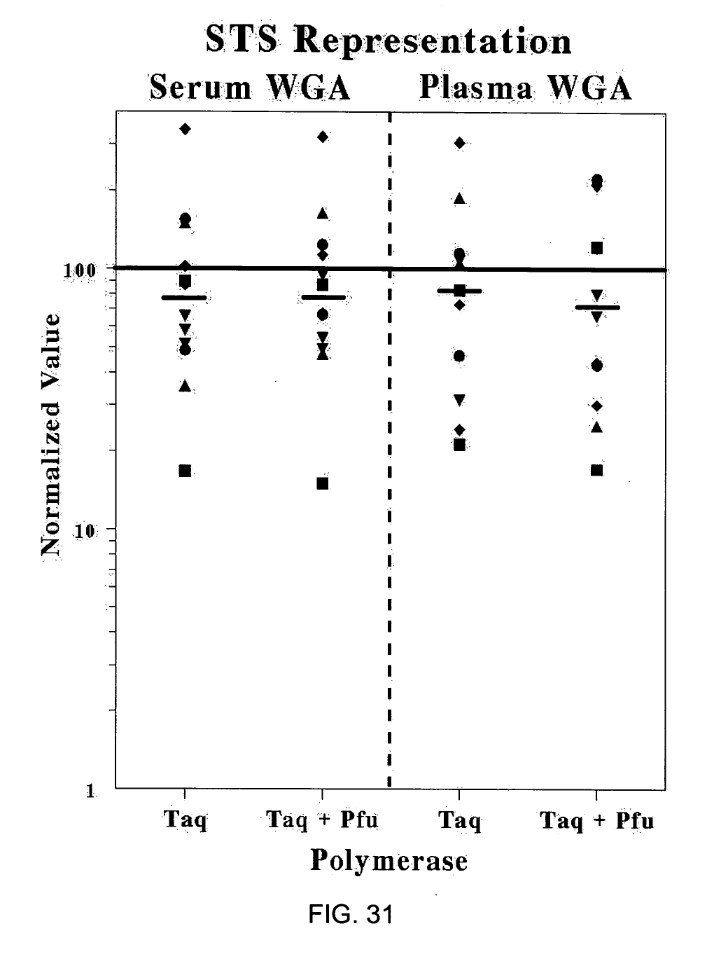

The amplified material was purified by Millipore Multiscreen PCR plates and quantified by optical density. Gel analysis of the amplified products (FIG. 30) indicated a size distribution (200 bp to 1 kb) similar to the original serum DNA. Additionally, the amplified DNA was analyzed using real-time, quantitative PCR using a panel of human genomic STS markers. The markers that make up the panel are listed in Table IV. Quantitative Real-Time PCR was performed using an I-Cycler Real-Time Detection System (Bio-Rad), as per the manufacturer's directions. Briefly, 25 μl reactions consisting of 1×PCR Buffer, 400 uM dNTP, 0.5×Titanium Taq, 200 nM primers, and 1:100,000 dilutions of fluorescein calibration dye and SYBR Green I were amplified for 40 cycles at 94° C. for 15 sec and 65° C. for 1 min. Standards corresponding to 10, 1, and 0.2 ng of fragmented DNA were used for each STS, quantities were calculated by standard curve fit for each STS (I-Cycler software, Bio-Rad) and were plotted as distributions. Quantitative real-time PCR of the serum DNA products demonstrated that all of the 16 markers were within a factor of 7 of the mean amplification, with 15 markers within a factor of 4 of the mean amplification in both the presence and the absence of Pfu. Analysis of the plasma samples indicated that all of the 12 markers were within a factor of 6 of the mean amplification. FIG. 31 is a scatterplot of the representation of the human genomic STS markers in the serum and plasma WGA products.

### Example 14

### Application of Single-Cell WGA for Detection and Analysis of Abnormal Cells

WGA amplified single-cell DNA can be used to analyze tissue cell heterogeneity on the genomic level. In the exemplary case of cancer diagnostics, it would facilitate the detection and statistical analysis of heterogeneity of cancer cells present in blood and/or biopsies. In the exemplary case of prenatal diagnostics, it would allow the development of non-invasive approaches based on the identification and genetic analysis of fetal cells isolated from blood and/or cervical smears. Analysis of DNA within individual cells could also facilitate the discovery of new cell markers, features, or properties that are usually hidden by the complexity and heterogeneity of the cell population.

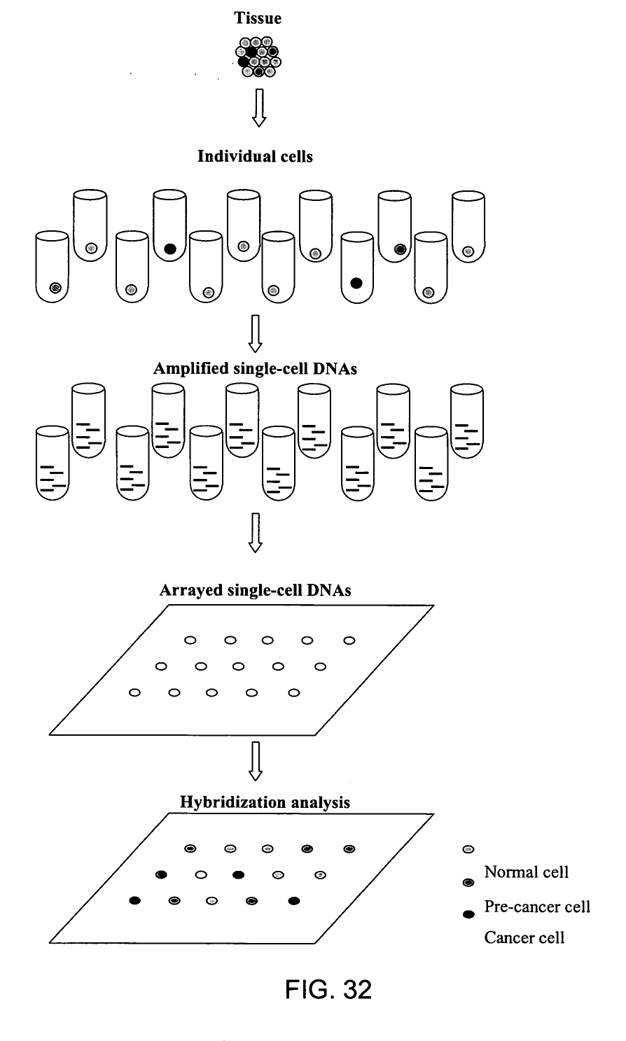

Analysis of the amplified single-cell DNA can be performed in two ways. In the approach shown in FIG. 32, amplified DNA samples are analyzed one by one using hybridization to genomic micro-array, or any other profiling tools such as PCR, sequencing, SNP genotyping, micro-satellite genotyping, etc. The method would include:

1. Dissociation of the tissue of interest into individual cells;

2. Preparation and amplification of individual (single-cell) WGA libraries;

3. Analysis of individual single-cell genomic DNA by conventional methods.

This approach can be useful in situations when genome-wide assessment of individual cells is necessary.

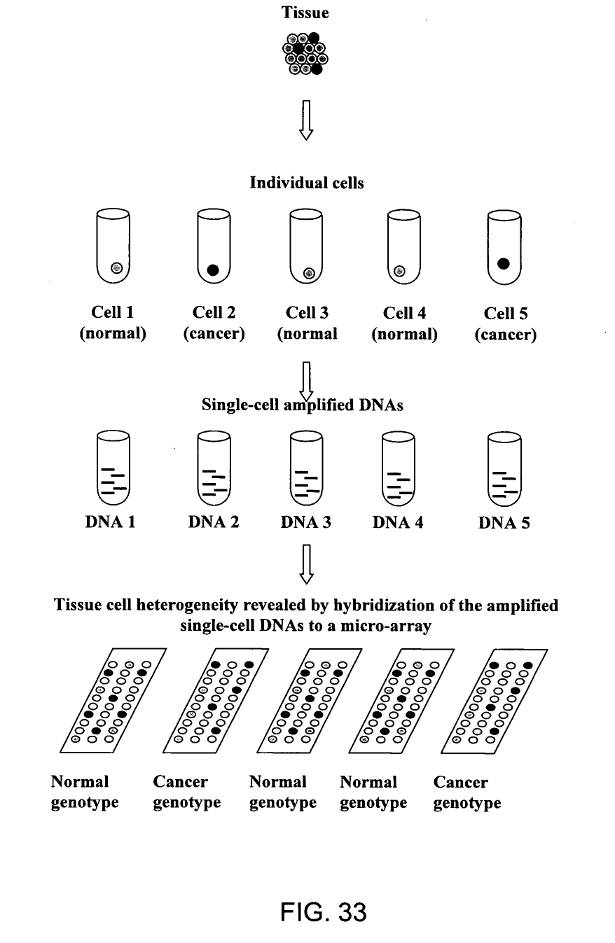

In the second approach, shown on FIG. 33, amplified DNA samples are spotted on the membrane, glass, or any other solid support, and then hybridized with a nucleic acid probe to detect the copy number of a particular genomic region. The method would include:

1. Dissociation of the tissue of interest into individual cells;

2. Preparation and amplification of individual (single-cell) WGA libraries;

3. Preparation of micro-arrays of individual (single-cell) WGA DNAs;

4. Hybridization of the single-cell DNA micro-arrays to a locus-specific probe; and

5. Quantitative analysis of the cell heterogeneity.

This approach can be especially valuable in situations when only a limited number of genomic regions should be analyzed in a large cell population.

### Example 15

### Whole Genome Amplification of Human Genomic DNA (50 Ng Template) Fragmented by Chemical Methods with Incorporation of DMSO and 7-deaza-DGTP during library Formation and Library Amplification

This example describes the amplification of 10 ng of genomic DNA that has been fragmented to an average size of 1 kb using chemical methods, specifically thermal fragmentation. The addition of the additives DMSO and 7-Deaza-dGTP during library preparation and/or library amplification improves the representation of GC rich regions of DNA that are often underrepresented.

Human DNA (50 ng) was diluted in TE to a final volume of 10 μl. The DNA was subsequently heated to 95° C. for 4′, and then cooled to 4° C. Two μl of 10×T4 DNA Ligase buffer was added to the DNA, and the mixture was heated to 95° C. for 10′, and then cooled to 4° C.

In order to generate competent ends for ligation, 40 nmol dNTP (Clontech), 0.1 pmol phosphorylated random nonamer primers (Genelink), and 5 U Klenow (NEB) were added in the presence or absence of either 4% DMSO (Sigma) and 3.4 nmol 7-Deaza-dGTP (Roche) or TE-Lo, and the resulting 17 μl reaction was incubated at 37° C. for 30′ and 12° C. for 1 h. Following incubation, the reaction was heated to 65° C. for 10′ to destroy the polymerase activity and then cooled to 4° C.

Universal adaptors were ligated to the template DNA by addition of the following reagents: 1 μl blunt end adaptor (10 pmol; FIG. 5A), 2 μl 5′ and 3′ overhang adaptors (10 pmol each; FIG. 5B), and 1 μl T4 DNA Ligase (400 Units, NEB) resulting in a final volume of 20 μl. The mixture was heated to 16° C. for 1 h and subsequently cooled to 4° C. The samples were diluted in TE-Lo to a final volume of 50 ul.

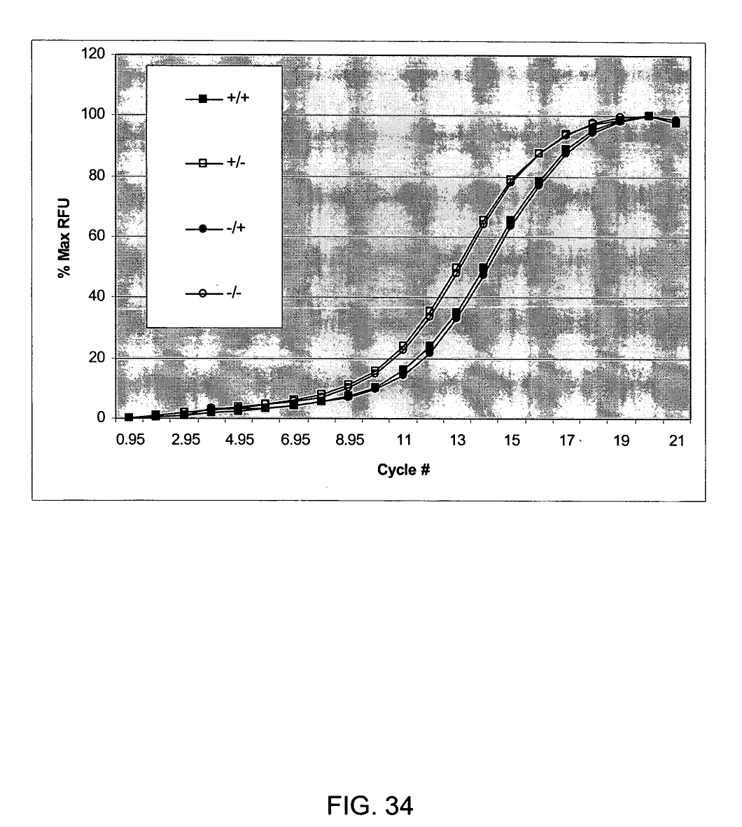

Extension of the 3′ end to fill in the universal adaptor and subsequent amplification of the library were carried out under the same conditions. Library (5 ng) was added to a 75 μl reaction containing 75 pmol T7 universal primer (SEQ ID NO:11), 120 nmol dNTP, 1×PCR Buffer (Clontech), and 1×Titanium Taq (Clontech) in the presence of 4% DMSO and 3.4 nmol 7-Deaza-dGTP, or TE-Lo. Fluorescein calibration dye (1:100,000) and SYBR Green I (1:100,000) were also added to allow monitoring of the reaction using real-time PCR (Bio-Rad). The samples were initially heated to 75° C. for 15′ to allow extension of the 3′ end of the fragments to fill in the universal adaptor sequence and displace the short, blocked fragment of the universal adaptor. Subsequently, amplification was carried out by heating the samples to 95° C. for 3′30″, followed by 22 cycles of 94° C. 15″, 65° C. 2′. The amplification curves depicted in FIG. 34 indicate that there is a 1 cycle delay in amplification when DMSO and 7-Deaza-dGTP are added during library amplification, but there is no effect when they are added during library preparation.

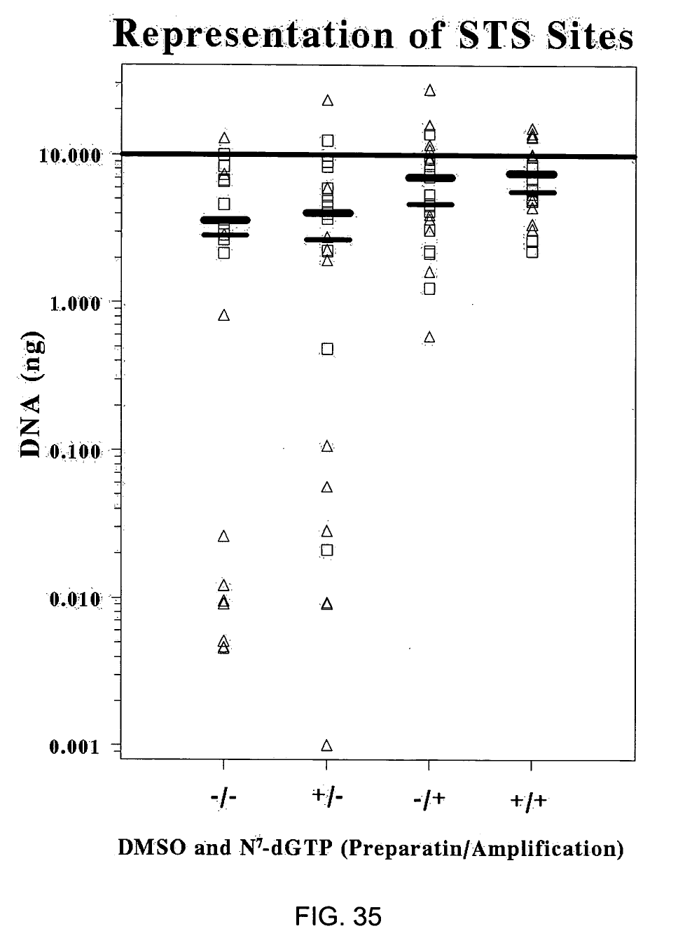

The amplified products were purified using the Qiagen Qiaquick purification system and the amount of amplified material was determined by optical density. Analysis of the amplified products using real-time PCR and 11 human genomic STS markers and 11 GC-rich genomic markers indicates that addition of DMSO and 7-Deaza-dGTP during both library preparation and amplification improves the representation of both the standard STS markers as well as the GC-rich markers (FIG. 35). When DMSO and 7-Deaza-dGTP are used in both library preparation and amplification, then all 22 sites were present within a factor of 4 of the mean amplification. The markers that make up the panel of 11 GC-rich genomic sites are listed in Table V, while the standard STS markers are listed in Table IV.

Library preparation using random hexamer primers in place of random nonamer primers resulted in similar amplification results (Data not shown).

TABLE V
HUMAN GC-RICH MARKERS USED FOR
REPRESENTATION ANALYSIS BY
QUANTITATIVE REAL-TIME PCR

| No* | Accession #** |
|---|---|
| 21 | AJ322533 |
| 22 | AJ322546 |
| 23 | AJ322610 |
| 27 | AJ322568 |
| 28 | AJ322570 |
| 29 | AJ322572 |
| 31 | AJ322623 |
| 35 | AJ322781 |
| 36 | AJ322715 |
| 37 | AJ322747 |
| 38 | AJ322801 |

*Omitted sequential numbers indicate dropped sequences that did not amplify well in quantitative RT-PCR
**Accession numbers of the GC-Rich marker sequences from the National Center for Biotechnology Information Entrez nucleotide database. Sequences of the regions as well as the forward and backward primers used in quantitative real-time PCR can be found in the Entrez nucleotide database at the National Center for Biotechnology Information's website.

### Example 16

### Incorporation of Poly-G and Poly-C Functional Tags Into WGA Libraries

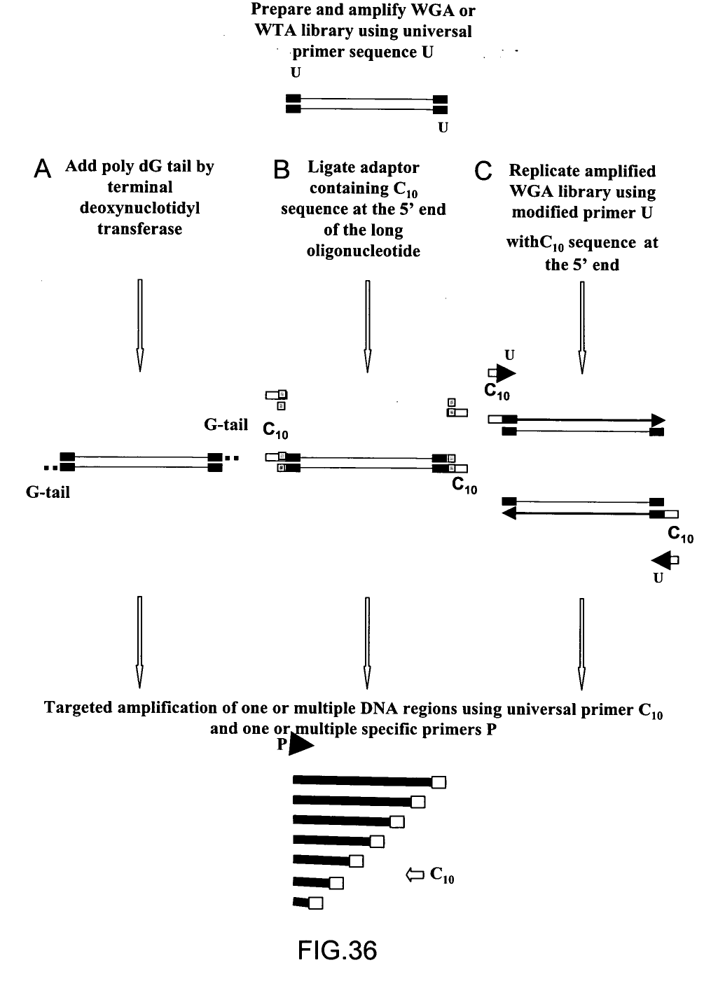

WGA libraries prepared by the method of library synthesis described in the invention may be modified or tagged to incorporate specific sequences. The tagging reaction may incorporate a functional tag. For example, the functional 5′ tag composed of poly cytosine may serve to suppress library amplification with a terminal C₁₀ sequence as a primer. Terminal complementary homo-polymeric G sequence can be added to the 3′ ends of amplified WGA library by terminal deoxynucleotidyl transferase (FIG. 36A), by ligation of adapter containing poly-C sequence (FIG. 36B), or by DNA polymerization with a primer complementary to the universal proximal sequence U with a 5′ non-complementary poly-C tail (FIG. 36C). The C-tail may be from 8-30 bases in length. In a preferred embodiment the length of C-tail is from 10 to

As described in U.S. Patent Application No. 20030143599, hereby incorporated in its entirety, genomic DNA libraries flanked by homo-polymeric tails consisting of G/C base paired double stranded DNA, or poly-G single stranded 3-extensions, are suppressed in their amplification capacity with poly-C primer. This suppression is caused by reduced priming efficiency in poly G regions because of formation of alternative G-quartet-like secondary structures within this sequence G-tail suppression is independent of the size of DNA amplicons, in contrast to well known “suppression PCR” that results from “pan-like” double-stranded structures formed by self-complementary adaptors which is strongly dependent on the size of DNA fragments being more prominent for short amplicons (Siebert et al., 1995; US005759822A). The G-tail suppression effect is diminished for a targeted site when balanced with a second site-specific primer, whereby amplification of a plurality of fragments containing the unique priming site and the universal terminal sequence are amplified selectively using a specific primer and a poly-C primer, for instance primer C₁₀. Those skilled in the art will recognize that genomic complexity may dictate the requirement for sequential or nested amplifications to amplify a single species of DNA to purity from a complex WGA library.

### Example 17

### Application of Homopolymeric G/C Tagged WGA Libraries for Targeted Dna Amplification

Targeted amplification may be applied to genomes for which limited sequence information is available or where rearrangement or sequence flanking a known region is in question. For example, transgenic constructs are routinely generated by random integration events. To determine the integration site, directed sequencing or primer walking from sequences known to exist in the insert may be applied. The invention described herein can be used in a directed amplification mode using a primer specific to a known region and a universal primer. The universal primer is potentiated in its ability to amplify the entire library, thereby substantially favoring amplification of product between the specific primer and the universal sequence, and substantially inhibiting the amplification of the whole genome library.

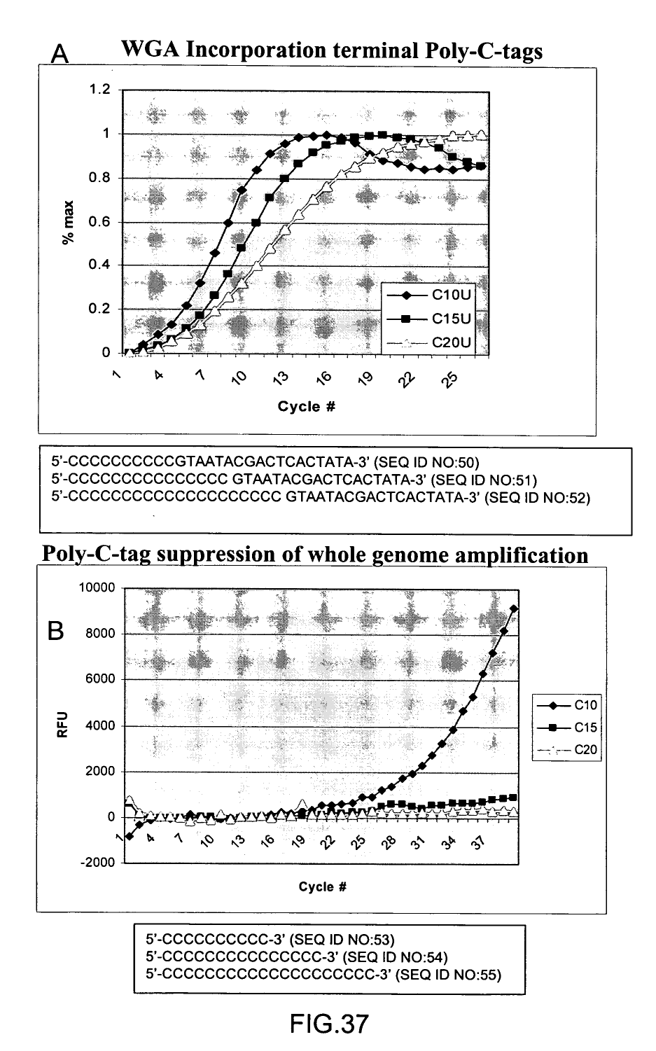

Conversion of WGA libraries for targeted applications involves incorporation of homo-polymeric G/C terminal tags. Amplification of libraries with C-tailed universal primers exhibit a dependence on the length of the 5′ poly-C extension component of the primer. WGA libraries prepared by the methods described in the invention can be converted for targeted amplification by PCR re-amplification using poly-C extension primers. FIG. 37A shows potentiated amplification with increasing length of poly-C in real-time PCR. The reduced slope of the curves for C₁₅U and C₂₀U show delayed kinetics and suggest reduced template availability or suppression of priming efficiency.

To demonstrate the suppression of library amplification imposed by poly-C tagging, libraries were purified using Qiaquick PCR purification column (Qiagen) and subjected to PCR amplification with poly-C primers corresponding to the length of their respective tag. FIG. 37B shows real-time PCR results that reflect the suppression of whole genome amplification. Only the short C₁₀ tagged libraries retain a modest amplification capacity, while C₁₅ and C₂₀ tags remain completely suppressed after 40 cycles of PCR.

### Example 18

### Application of Homopolymeric G/C Tagged WGA Libraries for Multiplexed Targeted Dna Amplification

Application of G/C tagged libraries for targeted amplification uses a single specific primer to amplify a plurality of library amplimers. The complexity of the target library dictates the relative level of enrichment for each specific primer. In low complexity bacterial genomes a single round of selection is sufficient to amplify an essentially pure product for sequencing or cloning purposes, however in high complexity genomes a secondary, internally “nested”, targeting event may be necessary to achieve the highest level of purity.

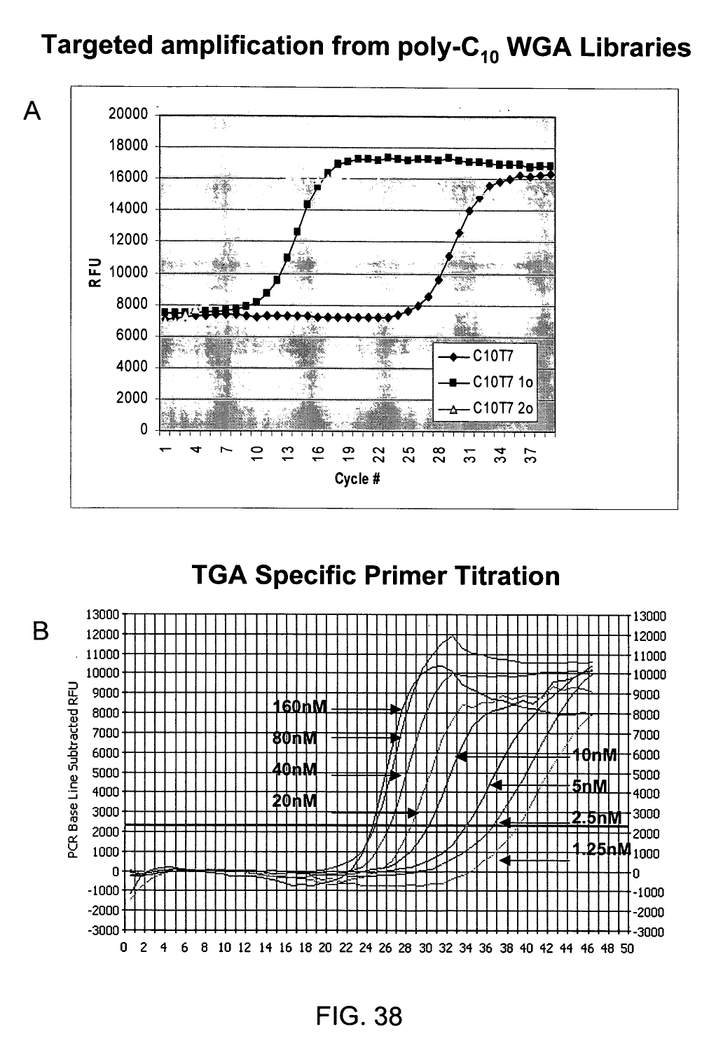

Using a human WGA library with C₁₀ tagged termini incorporated by re-amplification with C-tailed universal U primers, specific sites were targeted and the relative enrichment evaluated in real-time PCR. FIG. 38A shows the chromatograms from real-time PCR amplification for sequential primary 1° and secondary 2° targeting primers in combination with the universal tag specific primer C₁₀, or C₁₀ alone. The enrichment for this particular targeted amplicon achieved in the primary amplification is approximately 10,000 fold. Secondary amplification with a nested primer enriches to near purity with an additional two orders of magnitude for a total enrichment of 1,000,000 times the starting template. It is understood to those familiar with the art that enrichment levels may vary with primer specificity, while primers of high specificity applied in sequential targeted amplification reactions generally combine to enrich products to near purity.

To apply targeted amplification in a multiplexed format, specific primer concentrations were reduced 5 fold (from 200 nM to 40 nM) without significant loss of enrichment of individual sites (FIG. 38B). This primer concentration reduction allows for the combination of 45 specific primers and universal CIO primer to maintain total primer concentrations within reaction tolerances [2 μM].

To evaluate the utility of multiplex-targeted amplification, a set of primers were designed adjacent to STS sites (Table IV) using Oligo Version 6.53 primer analysis software (Molecular Biology Insights, Inc.: Cascade CO). Primers were 18-25 bases long, having high internal stability, low 3′-end stability, and melting temperatures of 57-62° C. (at 50 mM salt and 2 mM MgCl₂). Primers were designed to meet all standard criteria, such as low primer-dimer and hairpin formation, and are filtered against a human genomic database 6-mer frequency table. Primary multiplexed targeted amplification of G/C tagged WGA libraries was performed using 10-50 ng of tagged WGA library, 10-40 nM each of 45 specific primers (Table VI), 200 nM C₁₀ primer, dNTP mix, 1×PCR buffer and 1×Titanium Taq polymerase (Clontech), FCD (1:100,000) and SGI (1:100,000) dyes (Molecular Probes) added for real-time PCR detection using the I-Cycler (Bio-Rad). Amplification is carried out by heating the samples to 95° C. for 3′30″, followed by 18-24 cycles of 94° C. 20″, 68° C. 2′. The cycle number to reaction plateau is dependent on the absolute template and primer concentrations. The amplified material was purified by Qiaquick spin column (Qiagen), and quantified spectrophotometrically.

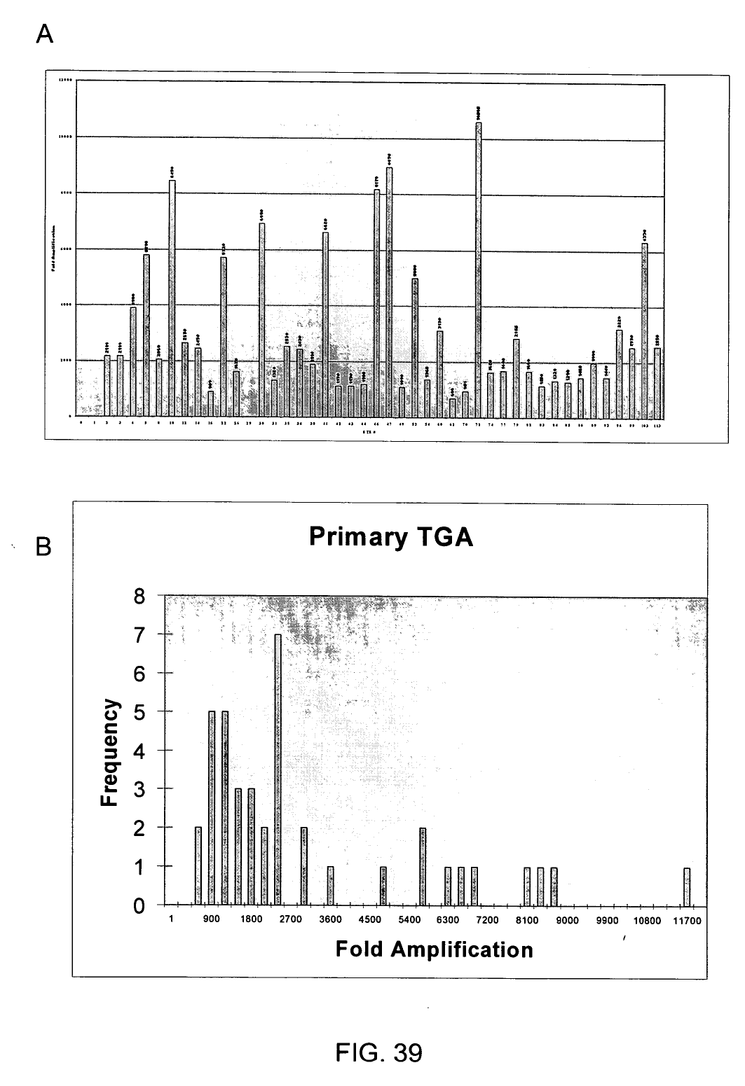

The enrichment of each site was evaluated using real-time PCR. Quantitative Real-Time PCR was performed using an I-Cycler Real-Time Detection System (Bio-Rad), as per the manufacturer's directions. Briefly, 25 μl reactions consisting of 1×PCR Buffer, 400 uM dNTP, 0.5×Titanium Taq, 200 nM primers, and 1:100,000 dilutions of fluorescein calibration dye and SYBR Green I were amplified for 40 cycles at 94° C. for 15 sec and 68° C. for 1 min. Standards corresponding to 10, 1, and 0.2 ng of fragmented DNA were used for each STS, quantities were calculated by standard curve fit for each STS (1-Cycler software, Bio-Rad) and were plotted as distributions. FIG. 39A shows the relative fold amplification for each targeted site. Primary amplification of sites 1 and 29 failed to amplify in multiplex reactions and displayed delayed kinetics in singlet reactions (not shown). A distribution plot of the same data shows an average enrichment of 3000 fold (FIG. 39B). Differences in enrichment level such as highly over-amplified sites are likely to arise from false priming elsewhere on the template. Such variation is compensated with the use of nested amplification of the enriched template.

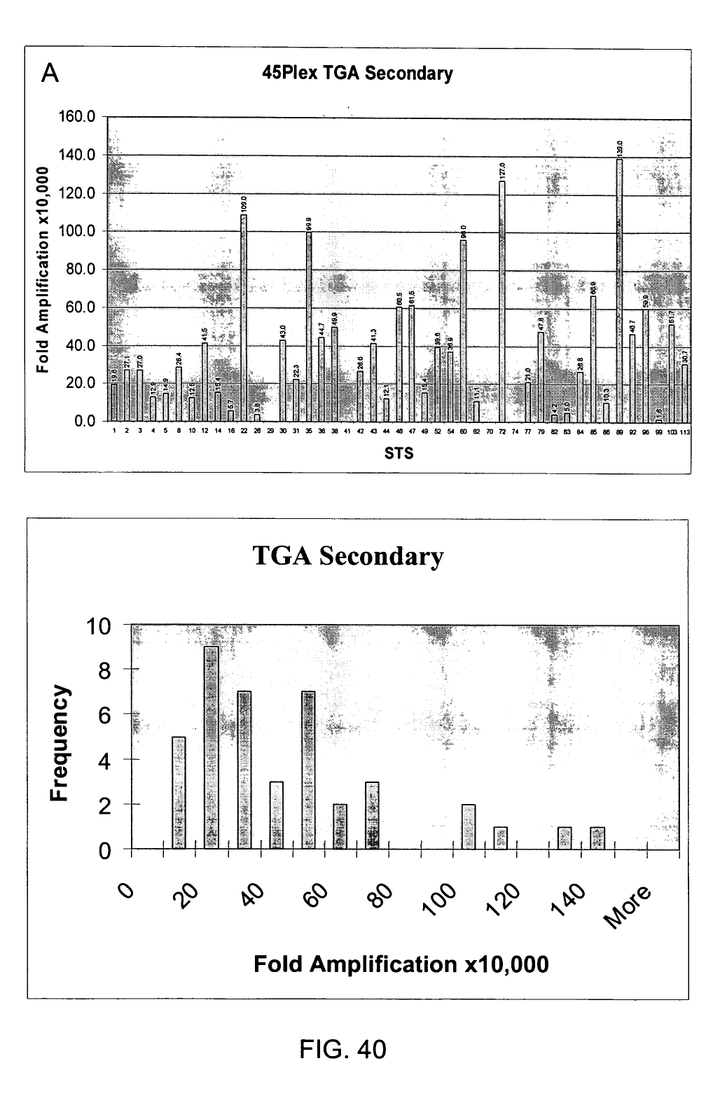

Secondary targeted amplifications were performed using primary targeting products as template and secondary nested primers (Table VI) in combination with the universal C₁₀ primer. Reactant concentrations and amplification parameters were identical to primary amplifications above. Multiplexed secondary amplifications were purified by Qiaquick spin column (Qiagen) and quantified by spectrophotometer. Enrichment of specific sites was evaluated in real-time PCR using an I-Cycler Real-Time Detection System (Bio-Rad), as per the manufacturer's directions. Briefly, 25 μl reactions consisting of 1×PCR Buffer, 400 uM dNTP, 0.5×Titanium Taq, 200 nM primers, and 1:100,000 dilutions of fluorescein calibration dye and SYBR Green I were amplified for 40 cycles at 94° C. for 15 sec and 68° C. for 1 min. Standards corresponding to 10, 1, and 0.2 ng of fragmented DNA were used for each STS, quantities were calculated by standard curve fit for each STS (1-Cycler software, Bio-Rad) and were plotted as distributions. FIG. 40A shows the relative abundance of each site after nested amplification and FIG. 40B plots the data in terms of frequency.

Targeted amplification applied in this format reduces the primer complexity required for multiplexed PCR. The resulting pool of amplimers can be evaluated on sequencing or genotyping platforms.

### Example 19

### Non-Redundant Genomic Sequencing of Unculturable Or Limited Species Facilitated by Whole Genome and Targeted Amplification

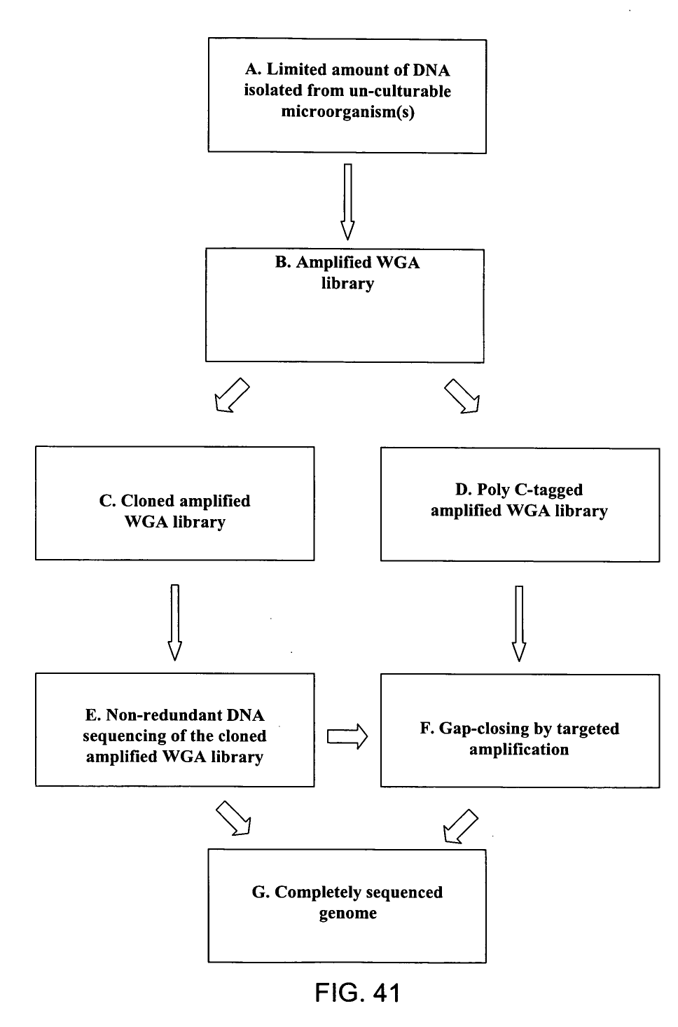

Whole genome and targeted amplification provide a unique opportunity for sequencing genomes of microorganisms that are difficult to grow or for species that are extinct. The diagram illustrating such a DNA sequencing application is shown in FIG. 41. First, limited amounts of DNA for the organism of interest (FIG. 41A) are converted into a WGA library using any method encompassed by the present invention, and amplified (FIG. 41B). Second, a fraction of amplified WGA DNA is cloned in a bacterial vector (FIG. 41C) while another fraction of amplified WGA DNA is converted into a C-tagged WGA library (FIG. 41D). Third, the cloned DNA is sequenced with minimal redundancy (FIG. 41E) to generate enough sequence information to initiate targeted sequencing and “walking” (FIG. 41F) that should ultimately result in sequencing of all gaps remaining after non-redundant sequencing and finishing of the sequencing application (FIG. 41G). The outlined strategy can be used not only for sequencing of limited material but also in any large DNA sequencing projects by replacing the costly and tedious highly redundant “shotgun” method.

TABLE VI
Targeted Amplification Primers

| Primary | | | Secondary | | |
|---|---|---|---|---|---|
| STS 1P | GCATATCCATATCTCCCGAAT | (SEQ ID NO:122) | STS 1S | TAAGCAGCAAGGTCTGGG | (SEQ ID NO:77) |
| STS 2P | CAGAGCACTCCAGACCATACG | (SEQ ID NO:123) | STS 2S | GTGATTGAACAATTTGGACCCAC | (SEQ ID NO:78) |
| STS 3P | CTTCGTTATGACCCCTGCTCC | (SEQ ID NO:124) | STS 3S | ATGGCAACATTCCACCTAGTAGC | (SEQ ID NO:79) |
| STS 4P | TCCCAAGATGAATGGTAAGACG | (SEQ ID NO:125) | STS 4S | CTCCGTCATGATAAGATGCAGT | (SEQ ID NO:80) |
| STS 5P | TCCAATCTCATCGGTTTACTG | (SEQ ID NO:126) | STS 5S | ACTGTTTGGGGTGTGAAAGGAC | (SEQ ID NO:81) |
| STS 8P | TCCAGAGCCCAGTAAACAACA | (SEQ ID NO:127) | STS 8S | ACTAACAACGCCCTTTGCTC | (SEQ ID NO:82) |
| STS 10P | TTACTTCAGCCCACATGCTTC | (SEQ ID NO:128) | STS 10S | TCAGCACTCCGTATCTTCATTTG | (SEQ ID NO:83) |
| STS 12P | TTCCGACATAGCGACTTTGTAG | (SEQ ID NO:129) | STS 12S | TAAACCGCTAAAACGATAGCAGC | (SEQ ID NO:84) |
| STS 14P | AAGGATCAGAGATACCCCACGG | (SEQ ID NO:130) | STS 14S | TCATGGTATTAGGGAAGTGGGAG | (SEQ ID NO:85) |
| STS 16P | TCCAAGAACCAACTAAGTCCAGA | (SEQ ID NO:131) | STS 16S | GGGAATGAAAAGAAAAGGCATTC | (SEQ ID NO:86) |
| STS 22P | CTAAGGGCAAACATAGGGATCAA | (SEQ ID NO:132) | STS 22S | TCTTTCCCTCTACAACCCTCTAACC | (SEQ ID NO:87) |
| STS 26P | CAACCTTTGAAGCCACTTTGAC | (SEQ ID NO:133) | STS 26S | CAGTACATGGGTCTTATGAGTAC | (SEQ ID NO:88) |
| STS 29P | GCCTCCGTCATTGGTATTTTCT | (SEQ ID NO:134) | STS 29S | AATCGAGAACGCACAGAGCAGA | (SEQ ID NO:89) |
| STS 30P | TGGCAACACGGTGCTGACCTG | (SEQ ID NO:135) | STS 30S | GTCTGGGGAGTAAATGCAACATC | (SEQ ID NO:90) |
| STS 31P | ATCATGGGTTTGGCAGTAAAGC | (SEQ ID NO:136) | STS 31S | TTCTTGATGACCCTGCACAA | (SEQ ID NO:91) |
| STS 35P | AGAACCAGCAAACCCAGTCCC | (SEQ ID NO:137) | STS 35S | CAGCAGAAGCACTACCAAAGACA | (SEQ ID NO:92) |
| STS 36P | GAAAGGGTGGATGGATTGAAA | (SEQ ID NO:138) | STS 36S | TTCACCTAGATGGAATAGCCACC | (SEQ ID NO:93) |
| STS 38P | TCAGATTTCCTGGCTCCGCTT | (SEQ ID NO:139) | STS 38S | GCAAGATTTTTGCTTGGCTCTAT | (SEQ ID NO:94) |
| STS 41P | CCTTCTGCTTCCCTGTGACCT | (SEQ ID NO:140) | STS 41S | GAATTTTGGTTTCTTGCTTTGG | (SEQ ID NO:95) |
| STS 42P | TGAACCCCACGAGGTGACAGT | (SEQ ID NO:141) | STS 42S | GTCAGAAGACTGAAAACGAAGCC | (SEQ ID NO:96) |
| STS 43P | GACATTACCAGCCCCTCACCTA | (SEQ ID NO:142) | STS 43S | CATCTCTTGATCATCCCAGCTCT | (SEQ ID NO:97) |
| STS 44P | TCCTTGACAGTTCCATTCACCA | (SEQ ID NO:143) | STS 44S | CACCATTGGTTGATAGCAAGGTT | (SEQ ID NO:98) |
| STS 46P | TTTGCAGGTAGCTCTAGGTCA | (SEQ ID NO:144) | STS 46S | TAAACATAGCACCAAGGGGC | (SEQ ID NO:99) |
| STS 49P | CCCAGAAACCCTGAGACCCTC | (SEQ ID NO:56) | STS 49S | CGTCTCTCCCAGCTAGGATG | (SEQ ID NO:101) |
| STS 52P | TGTGCCACAAGTTAAGATGCT | (SEQ ID NO:57) | STS 52S | CTTTTTCACAGAACTGGTGTCAGG | (SEQ ID NO:102) |
| STS 54P | TGCTGTATCGTGCCTGCTCAAT | (SEQ ID NO:58) | STS 54S | ACCCAGCTTTCAGTGAAGGA | (SEQ ID NO:103) |
| STS 60P | TGCCCCACTCCCCAACATTCT | (SEQ ID NO:59) | STS 60S | AATCAAAAGGCCAACAGTGG | (SEQ ID NO:104) |
| STS 62P | AACAGAGCCTCAGGGACCAGT | (SEQ ID NO:60) | STS 62S | ACTGGCTGAGGGAGCATG | (SEQ ID NO:105) |
| STS 70P | GGGCTTTGTCTGTGGTTGGTA | (SEQ ID NO:61) | STS 70S | TAAATGTAACCCCCTTGAGCC | (SEQ ID NO:106) |
| STS 72P | TGGGCTGGCTGAGGTCAAGAT | (SEQ ID NO:62) | STS 72S | TATTGACCACATGACCCCCGT | (SEQ ID NO:107) |
| STS 74P | TTTTGCTCCGCTGACATTTGG | (SEQ ID NO:63) | STS 74S | TTGGGTGATGTCTTCACATGG | (SEQ ID NO:108) |
| STS 77P | TGCTCCTGTCCCTTCCACTTC | (SEQ ID NO:64) | STS 77S | GCTCAATAAAAATAGTACGCCC | (SEQ ID NO:109) |
| STS 79P | CCTTATTCCCAGCAGCAGTATTC | (SEQ ID NO:65) | STS 79S | TTCTCCCAGCTTTGAGACGT | (SEQ ID NO:110) |
| STS 82P | TGGGAAGGGAAAGAGGGTACT | (SEQ ID NO:66) | STS 82S | TTTGTTACTTGCTACCCTAG | (SEQ ID NO:111) |
| STS 83P | TTGCTGTAGATGGGCTTTCGT | (SEQ ID NO:67) | STS 83S | GAAGATGAAGTGAACTCCTATCC | (SEQ ID NO:112) |
| STS 85P | GGCACAAGCAAAAGGGTGTCT | (SEQ ID NO:69) | STS 85S | ATGTTTCTCTGGCCCCAAG | (SEQ ID NO:113) |
| STS 89P | CACCTGTCTTGTTGGCATCACC | (SEQ ID NO:71) | STS 89S | TTGGGAAATGTCAGTGACCA | (SEQ ID NO:114) |
| STS 92P | TTGTTTTGCCTCACCAGTCATTT | (SQ ID NO:72) | STS 92S | TGTGGTTAGGATAGCACAAGCATT | (SQ ID NO:117) |
| STS 96P | TCAGCAAACCCAAAGATGTTA | (SEQ ID NO:73) | STS 96S | TGCAATTTGAAGGTACGAGTAG | (SEQ ID NO:118) |
| STS 99P | TTAGTCCTTTGGGCAGCACGA | (SEQ ID NO:74) | STS 99S | TGTTAACAATTTGCATAACAAAAGC | (SQ ID NO:119) |
| STS 103P | TGTCTCTGCTTCTGAAACGGG | (SEQ ID NO:75) | STS 103S | GCATTTTCTGTCCCACAAGATATG | (SEQ ID NO:120) |
| STS 113P | ACTGCCAGGGTCATTGACTT | (SEQ ID NO:76) | STS 113S | ATTGCTGTCACAGCACCTTG | (SEQ ID NO:121) |

*P - denotes primary targeted amplification primer
*S - denotes secondary targeted amplification primer

### Example 20

### Creation and Amplification of a Secondary Genome Library by incorporation of a homopolymeric sequence to a Primary whole genome library, digestion with a nuclease, Attachment of a Second Universal Adaptor, and Amplification With Primers Complementary to the Homopolymeric Tail and the Second adaptor.

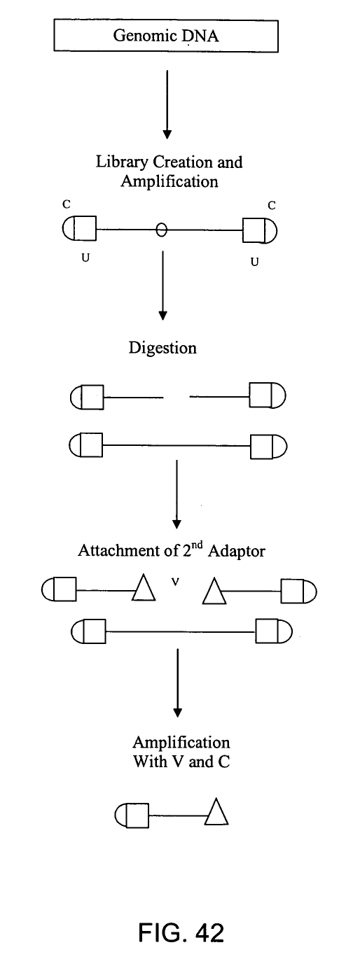

The method described in this Example presents a method for the generation of a secondary genome library containing regions of interest contained within the primary whole genome library. FIG. 42 is a depiction of this protocol. Genomic DNA is converted into a primary whole genome library, containing universal adaptor U, and amplified. A homopolymeric C-tail (C) is added to the 5′ end of the libraries during either library preparation or amplification. This addition is described in Example 16 and depicted in FIG. 36. Following amplification of the primary whole genome library, the amplicons are digested with a nuclease targeted at specific sites, for example a methylation-sensitive restriction endonuclease. Following digestion, a second adaptor (V) is attached to the ends of the molecules resulting from digestion to create the secondary library. Amplification of the secondary library with primers V and C results only in amplification of molecules containing primer C at one end and primer V at the other end, or molecules containing primer V at both ends. Molecules containing primer C at both ends are not amplified due to the nature of the homopolymeric C-tail sequence. The resulting amplified library is highly enriched in the sequences of interest and can be analyzed by a variety of means known in the art, including PCR, microarray hybridization, and probe assay.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference.

### PATENTS

U.S. Pat. No. 4,683,195

U.S. Pat. No. 4,683,202

U.S. Pat. No. 4,800,159

U.S. Pat. No. 4,883,750

U.S. Pat. No. 5,648,245

U.S. Pat. No. 5,759,822

U.S. Pat. No. 5,882,864

U.S. Pat. No. 6,107,023

U.S. Pat. No. 6,114,149

U.S. Pat. No. 6,280,949

U.S. patent application Ser. No. 10/293,048

U.S. Patent Application No. 60/453/060

U.S. Patent Publication No. US 2003/0013671

PCT Patent Application No. PCT/US87/00880

PCT Patent Application No. PCT/US89/01025

PCT Patent Application No. PCT/US02/37322

PCT Patent Application No. WO 88/10315

PCT Patent Application No. WO 89/06700

PCT Patent Application No. WO 00/17390

PCT Patent Application No. WO 90/07641

British Patent Application No. GB 2,202,328

European Patent No. 320,308

Japan Patent No. JP8173164A2

### PUBLICATIONS

Allsopp, R. C., Chang, E., Kashefi-aazam, M., Rogaev, E. I., Piatyszek, M. A., Shay, J. W. and Harley, C. B. 1995. Telomere shortening is associated with cell division in vitro and in vivo. Exp. Cell Res., 220:194-200.

Allsopp, R. C., Vaziri, H., Patterson, C., Goldstein, S., Younglai, E. V., Futcher, A. B., Greider, C. W. and Harley, C. B. 1992. Telomere length predicts replicative capacity of human fibroblasts. Proc. Natl. Acad. Sci. USA, 89:10114-10118.

Anderson, S. 1981. Shotgun DNA sequencing using cloned DNase I-generated fragments. Nucleic Acids Res., 9:3015-5027.

Ausubel, F. M., Brent, R., Kingston, R. E., Moore, D. O., Seidman, J. S., Smith, J. A., and Struhl, K. 1987. Current protocols in molecular biology. Wiley, New York, N. Y.

Bankier, A. T. 1993. Generation of random fragments by sonication. Methods Mol. Biol., 23:47059.

Bodenteich, A., Chissoe, S. L., Wang, Y.-F., and Roe, B. A. 1994. Shotgun cloing or the strategy of choice to generate template for high-throughput dideoxynucleotide sequencing. In: Automated DNA sequencing and analysis (ed. M. D. Adams, C. Fields, and J. C. Venter), pp. 42-50. Academic Press, San Diego, Calif.

Bodnar, A. G., Ouellette, M., Frolkis, M., Holt, S. E., Chiu, C.-P., Morin, G. B., Harley, C. B., Shay, J. W., Lichtsteiner, S. and Wright W. E. 1998. Extension of life-span by introduction of telomerase into normal human cells. Science, 279:349-352.

Bohlander, S. K., Espinosa, R., LeBeau, M. M., Rowler, J. D., Diaz, M. O. 1992. A method for the rapid sequence-independent amplification of microdissected chromosomal material. Genomics, 13:1322-1324.

Bond, J., Haughton, M., Blaydes, J., Gire, V., Wynfordthomas, D. and Wyllie, F. 1996. Evidence that transcriptional activation by p53 plays a direct role in the induction of cellular senescence. Oncogene, 13:2097-2104.

Branum, M. E., Tipton, A. K., Zhu, S., and Que, L. Jr. 2001. Double-strand hydrolysis of plasmid DNA by dicerium complexes at 37 degrees C. J. Am. Chem. Soc., 123:1898-1904.

Buchanan, A. V., Risch, G. M., Robichaux, M., Sherry, S. T., Batzer, M. A., Weiss, K. M. 2000. Long DOP-PCR of rare archival anthropological samples. Hum. Biol., 72:911-925.

Chang, K. S., Vyas, R. C., Deaven, L. L., Trujillo, J. M., Stass, S. A., Hittelman W. N. 1992. PCR amplification of chromosome-specific DNA isolated from flow cytometry-sorted chromosomes. Genomics, 12:307-312.

Cheng, J., Waters, L. C., Fortina, P., Hvichia, G., Jacobson, S. C., Ramsey, J. M., Kricka, L. J., Wilding, P. 1998. Degenerate oligonucleotide-primed polymerase chain reaction and capillary electrophoretic analysis of human DNA on a microchip-based devices. Anal. Biochem., 257:101-106.

Cheung, V. G., Nelson, S. F. 1996. Whole genome amplification using a degenerate oligonucleotide primer allows hundreds of genotypes to be performed on less than one nanogram of genomic DNA. Proc. Natl. Acad. Sci. USA, 93:14676-14679.

Coligan, J. E., Kruisbeek A. M., Margulies, D. H., Shevach, E. M., Strober, W. 1991. Current protocols in immunology. John Wiley and Sons, Hoboken, N.J.

Counter, C. M., Avilion, A. A., LeFeuvre, C. E., Stewart, N. G., Greider, C. W., Harley, C. B. and Bacchetti, S. 1992. Telomere shortening associated with chromosome instability is arrested in immortal cells which express telomerase activity. EMBO J., 11:1921-1929.

Dean, F., Nelson, J., Giesler, T., Lasken, R. 2001. Rapid amplification of plasmid and phage DNA using φ29 DNA polymerase and multiply-primed rolling circle amplification. Genome Res., 11:1095-1099.

Dean, F., Hosono, S., Fang, L., Wu, X., Faruqi, A. F., Bray-Ward, P., Sun, Z., Zong, Q., Du, Y., Du, J., Driscoll, M., Song, W., Kingsmore, S., Egholm, M., Lasken, R. S. 2002. Comprehensive human genome amplification using multiple displacement amplification. Proc. Natl. Acad. Sci. USA, 99:5261-5266.

Di Leonardo, A., Linke, S. P., Clarkin, K. and Wahl, G. M. 1994. DNA damage triggers a prolonged p53-dependent G1 arrest and long-term induction of Cip1 in normal human fibroblasts. Genes Dev., 8:2540-2551.

Dietmaier, W., Hartmann, A., Wallinger, S., Heinmöller, E., Kerner, T., Endl, E., Jauch, K. W., Hofstädter, F., Rüschoff, J. 1999. Multiple mutation analyses in single tumor cells with improved whole genome amplification. Am. J. Path., 154:83-95.

Doolittle, R. 1990. Methods in Enzymology. Academic Press, San Diego.

Franklin, S. J. 2001. Lanthanide-mediated DNA hydrolysis. Curr. Opin. Chem. Biol., 5:201-208.

Freshney, R. I. 1987. Culture of animal cells: a manual of basic technique, 2d ed., Wiley-Liss, London.

Frohman, M. A. 1990. Race: Rapid amplification of cDNA ends. In Innis, M. A., Gelfand, D. H., Sninsky, J. J., and White, T. J. eds., PCR protocols. Academic press, New York. Pp 28-38.

Gait, M. 1984. Oligonucleotide Synthesis. Practical Approach Series. IRL Press, Oxford, U. K.

Gingrich, J. C., Boehrer, D. M., Basu, S. B. 1996. Partial CviJI digestion as an alternative approach to generate cosmid sublibraries for large-scale sequencing projects. Biotechniques, 21:99-104.

Grothues, D., Cantor, C. R., Smith, C. L. 1993. PCR amplification of megabase DNA with tagged random primers (T-PCR). Nucleic Acids Res., 21:1321-1322.

Hadano, S., Watanabe, M., Yokoi, H., Kogi, M., Kondo, I., Tsuchiya, H., Kanazawa, I., Wakasa, K., Ikeda, J. E. 1991. Laser microdissection and single unique primer PCR allow generation of regional chromosome DNA clones from a single human chromosome. Genomics, 11:364-373.

Hara, E., Smith, R., Parry, D., Tahara, H. and Peters, G. 1996. Regulation of p16 (CdkN2) expression and its implications for cell immortalization and senescence. Mol. Cell. Biol., 16:859-867.

Hayes, J. J., Kam, L., and Tullius, T. D. 1990. Footprinting protein-DNA complexes with gamma-rays. Methods Enzymol. 186:545-549.

Hayflick, L. and Moorhead, P. S. 1961. The serial cultivation of human diploid cell strains. Exp. Cell Res., 25:585-621.

Hayflick, L. 1965. The limited in vitro lifetime of human diploid cell strains. Exp. Cell Res., 37:614-636.

Hiyama, E., Tatsumoto, N., Kodama, T., Hiyama, K., Shay, J. W. and Yokoyama, T. 1996. Telomerase activity in human intestine. Int. J. Oncol., 9:453-458.

Innis, M. A., Gelfand, D. H., Sninsky, J. J. and White, T. J. 1990. PCR Protocols. Academic Press, New York.

Jiang, X. R., Jimenez, G., Chang, E., Frolkis, M., Kusler, B., Sage, M., Beeche, M., Bodnar, A. G., Wahl, G. M., Tlsty, T. D. and Chiu, C. P. 1999. Telomerase expression in human somatic cells does not induce changes associated with a transformed phenotype. Nature Genet., 21:111-114

Johnson, D. H. 1990. Molecular cloning of DNA from specific chromosomal regions by microdissection and sequence-independent amplification of DNA. Genomics, 6:243-251.

Kao, F. T., Yu, J. W. 1991. Chromosome microdissection and cloning in human genome and genetic disease analysis. Proc. Natl. Acad. Sci. USA, 88:1844-1848.

Kinzler, K. W., Vogelstein, B. 1989. Whole genome PCR: application to the identification of sequences bound by gene regulatory proteins. Nucleic Acid Res., 17:3645-3653.

Kittler, R., Stoneking, M., Kayser, M. 2002. A whole genome amplification method to generate long fragments from low quantities of genomic DNA. Anal. Biochem., 300:237-244.

Klein, C. A., Schmidt-Kittler, O., Schardt, J. A., Pantel, K., Speicher, M. R., Riethmjiller, G. 1999. Comparative genomic hybridization, loss of heterozygosity, and DNA sequence analysis of single cells. Proc. Natl. Acad. Sci. USA, 96:4494-4499.

Kleyn, P. W., Wang, C. H., Lien, L. L., Vitale, E., Pan, J., Ross, B. M., Grunn, A., Palmer, D. A., Warburton, D., Brzustowicz, L. M. 1993. Construction of yeast artificial chromosome contig spanning the spinal muscular atrophy disease gene region. Proc. Natl. Acad. Sci., 90:6801-6805.

Ko, M. S. H., Ko, S. B. H., Takahashi, N., Nishiguchi, K., Abe, K. 1990. Unbiased amplification of highly complex mixture of DNA fragments by ‘lone linker’-tagged PCR. Nucleic Acids Res., 18:4293-4294.

Komiyama, M., and Sumaoka, J. 1998. Progress towards synthetic enzymes for phosphoester hydrolysis. Curr. Opin. Chem. Biol., 2:751-757.

Korenburg, J. R., Rykowski, M. C. 1988. Human genome organization: Alu, LINES, and the molecular structure of metaphase chromosome bands. Cell, 53:391-400.

Kwoh, D. Y., Davis, G. R., Whitfield, K. M., Chappelle, H. L., DiMichele, L. J., and Gingeras, T. R. 1989. Transcription-based amplification system and detection of amplified human immunodeficiency virus type 1 with a bead-based sandwich hybridization format.

Lisitsyn, N., Lisitsyn, N., and Wigler, M. 1993. Cloning the differences between two complex genomes. Science, 259:946-951.

Lucito, R., Nakimura, M., West, J. A., Han, Y., Chin, K., Jensen, K., McCombie, R., Gray, J. W., and Wigler, M. 1998. Genetic analysis using genomic representations. Proc. Natl. Acad. Sci. USA, 95:4487-4492.

Lüdecke, H. J., Senger, G., Claussen, U., Horsthemke, B. 1989. Cloning defined regions of human genome by microdissection of banded chromosomes and enzymatic amplification. Nature, 338:348-350.

Martin, G. M., Sprague, C. A. and Epstein, C. J. 1970. Replicative lifespan of cultivated human cells: effect of donor's age, tissue and genotype. Lab. Invest., 23:86-92.

Milan, D., Yerle, M., Schmitz, A., Chaput, B., Vaiman, M., Frelatm, G., Gellin, J. 1993. A PCR-base method to amplify DNA with random primers: Determining the chromosomal content of porcine flow-karyotype peaks by chromosome painting. Cytogenet. Cell Genet., 62:139-141.

Miller, J. M., and Calos, M. P. 1987. Gene Transfer Vectors for Mammalian Cells. Cold Spring Harbor Laboratory, Cold Spring Harbor.

Miyashita, K., Vooijs, M. A., Tucker, J. D., Lee, D. A., Gray, J. W., Pallavicini, M. G. 1994. A mouse chromosome 11 library generated from sorted chromosomes using linker-adapter polymerase chain reaction. Cytogenet. Cell Genet., 66:54-57.

Morales, C. P., Holt, S. E., Ouellette, M., Kaur, K. J., Yan, Y., Wilson, K. S., White, M. A., Wright, W. E. and Shay, J. W. 1999. Lack of cancer-associated changes in human fibroblasts immortalized with telomerase. Nature Genet., 21:115-118.

Naylor, J., Brinke, A., Hassock, S., Green, P. M., Giannelli, F. 1993. Characteristic mRNA abnormality found in half the patients with sever hemophilia A is due to large DNA inversions. Hum. Mol. Genet., 2:1773-1778.

Nelson, D. G., Ledbetter, S. A., Corbo, L., Victoria, M. F., Ramirez-Solis, R., Webster, T. D., Ledbetter, D. H., Caskey, C. T. 1989. Alu polymerase chain reaction: A method for rapid isolation of human-specific sequences fro complex DNA sources. Proc. Natl. Acad. Sci. USA, 86:6686-6690.

Oefner, P. J., Hunicke-Smith, S. P., Chiang, L., Dietrich, F., Mulligan, J. And Davis, R. W. 1996. Efficient random subcloning of DNA sheared in a recirculating point-sink flow system. Nucleic Acids Res., 24:3879-3886.

Ohara O., Dorit, R. L., and Gilbert, W. 1989. One-sided polymerase chain reaction: the amplification of cDNA. Proc. Natl. Acad. Sci. USA, 86:5673-5677.

Olovnikov, A. M. 1973. A theory of marginotomy. The incomplete copying of template margin in enzymic synthesis of polynucleotides and biological significance of the phenomenon. J. Theor. Biol., 41:181-190.

Paunio, T., Reima I., Syvänen, A. C. 1996. Preimplantation diagnosis by whole-genome amplification, PCR amplification, and solid-phase minisequencing of blastomere DNA. Mol. Path. Genet., 42:1382-1390.

Price, M. A., and Tullius, T. D. 1992. Using hydroxyl racidal to probe DNA structure. Methods Enzymol., 212:194-219.

Ramirez, R. D., Wright, W. E., Shay, J. W. and Taylor, R. S. 1997. Telomerase activity concentrates in the mitotically active segments of human hair follicles. J. Invest. Dermatol., 108:113-117.

Richards, O. C., and Boyer, P. D., 1965. Chemical mechanism of sonic, acid, alkaline and enzymatic degradation of DNA. J. Mol. Biol. 11:327-340.

Riley, J., Butler, R., Ogilvie, D., Finniear, R., Jenner, D., Powell, S., Smith, J. C., Markham, A. F. 1990. A novel, rapid method for the isolation of terminal sequences from yeast artificial chromosome (YAC) clones. Nucleic Acids Res., 18:2887-2890.

Robles, S. J. and Adami, G. R. 1998. Agents that cause DNA double strand breaks lead to p16-ink4a enrichment and to premature senescence of normal fibroblasts. Oncogene, 6:1113-1123.

Roots, R., Holley, W., Chatteijee, A., Rachal, E., and Kraft, G. 1989. The influence of radiation quality on the formation of DNA breaks. Adv. Space Res., 9:45-55.

Sambrook, J., Fritsch, E. F. and Maniatis, T. (1989) Molecular Cloning: A Laboratory Manual, second edition, Cold Spring Harbor Laboratory, Cold Spring Harbor.

Sanchez-Cespedes, M., Cairns, P., Jen, J., Sidransky, D. 1998. Degenerate oligonucleotide-primed PCR (DOP-PCR): Evaluation of its reliability for screening of genetic alteration in neoplasia. Biotechniques, 25:1036-1038.

Saunders, R. D. C., Glover, D. M., Ashburner, M., Siden-Kiamos, I., Louis, C., Monastirioti, M., Savakis, C., Kafatos, F. 1989. PCR amplification of DNA microdissected from a single polytene chromosome band: A comparison with conventional microcloning. Nucleic Acids Res., 17:9027-9037.

Shay, J. W., Pereira-Smith, O. M. and Wright, W. E. 1991. A role for both RB and p53 in the regulation of human cellular senescence. Exp. Cell Res., 196:33-39.

Shay, J. W., Van Der Haegen, B. A., Ying, Y. and Wright, W. E. 1993. The frequency of immortalization of human fibroblasts and mammary epithelial cells transfected with SV40 large T-antigen. Exp. Cell Res., 209:45-52.

Siebert, P. D., Chenchik, A., Kellogg, D. E., Lukyanov, K. A., Lukyanov, S. A. 1995. An improved PCR method fpr walking in uncloned genomic DNA. Nucleic Acids Res., 23:1087-1088.

Telenius, H., Carter, N. P., Bebb, C. E., Nordenskjøld, M., Ponder, B. A. J., Tunnacliffe, A. 1992. Degenerate oligonucleotide-primed PCR: General amplification of target DNA by a single degenerate primer. Genomics, 13:718-725.

Thorstenson, Y. R., Hunicke-Smith, S. P., Oefner, P. J., and Davis, R. W. 1998. An automated hydrodynamic process for controlled, unbiased DNA shearing. Genome Res., 8:848-855.

Tullius, T. D. 1991. DNA footprinting with the hydroxyl racidal. Free Radic. Res Commun., 12-13:521-529.

Ulaner, G. A. and Giudice, L. C. 1997. Developmental regulation of telomerase activity in human fetal tissues during gestation. Mol. Hum. Reprod., 3:769-773.

Valdes, J. M., Tagle, D. A., Collins, F. S. 1994. Island rescue sequences from yeast artificial chromosomes and cosmids. Proc. Natl. Acad. Sci. USA, 91:5377-5381.

VanDevanter, D. R., Choongkittawom, N. M., Dyer, K. A., Aten, J., Otto, P., Behler, C., Bryant, E. M., Rabinovitch, P. S. 1994. Pure chromosome-specific PCR libraries from single sorted chromosome. Proc. Natl. Acad. Sci. USA, 91-5858-5862.

Vaziri, H. and Benchimol, S. 1996. From telomere loss to p53 induction and activation of a DNA-damage pathway at senescence: the telomere loss/DNA damage model of cell aging. Exp. Gerontol., 31:295-301.

Vaziri, H. and Benchimol, S. 1998. Reconstitution of telomerase activity in normal human cells leads to elongation of telomeres and extended replicative life span. Curr. Biol., 8:279-282.

Vooijs, M., Yu, L. C., Tkachuk, D., Pinkel, D., Johnson, D., Gray, J. W. 1993. Libraries for each human chromosome, constructed from sorter-enriched chromosomes by using linker-adaptor PCR. Am. J. Hum. Genet., 52:586-597.

Walker, G. T., Frasier, M. S., Schram, J. L., Little, M. C., Nadeau, J. G., and Malinowski, D. P. 1992. Strand displacement amplification-an isothermal, in vitro DNA amplification technique. Nucleic Acids Res., 20:1691-1696.

Watson, J. D. 1972. Origin of concatemeric T4 DNA. Nature, 239:197-201.

Weir, D. M. 1978. Handbook of Experimental Immunology. Blackwell Scientific Publications, Oxford, U. K.

Wells, D., Sherlock, J. K., handyside, A. H., Delhanty, J. D. A. 1999. Detailed chromosomal and molecular genetic analysis of single cells by whole genome amplification and comparative genomic hybrindisation. Nucleic Acids Res., 27:1214-1218.

Wesley, C. S., Ben M., Kreitman, M., Haga, N., Easnes, W. F. 1990. Cloning regions of the Drosophila genome by microdissection of polytene chromosome DNA and PCR with nonspecific primer. Nucleic Acids Res., 18:599-603.

Wong, K. K., Stillwell, L. C.-, Dockery, C. A., Saffer, J. D. 1996. Use of tagged random hexamer amplification (TRHA) to clone and sequence minute quantities of DNA-applications to a 180 kb plasmid from Sphingomonas F199. Nucleic Acids Res., 24:3778-3783.

Wright, W. E., Piatyszek, M. A., Rainey, W. E., Byrd, W. and Shay, J. W. 1996. Telomerase activity in human germline and embryonic tissues and cells. Dev. Genet., 18:173-179.

Wright, W. E. and Shay, J. W. 1992. The two-stage mechanism controlling cellular senescence and immortalization. Exp. Gerontol., 27:383-389.

Wu, D. Y., and Wallace R. B. 1989. The ligation amplification reaction (LAR)-amplification of specific DNA sequences using sequential rounds of template-dependent ligation. Genomics, 4:560-569.

Yui, J., Chiu, C. P. and Lansdorp, P. M. 1998. Telomerase activity in candidate stem cells from fetal liver and adult bone marrow. Blood, 91:3255-3262.

Zhang, L., Cui, X., Schmitt, K., Hubert, R., Navidi, W., Arnheim, N. 1992. Whole genome amplification from a single cell: Implications for genetic analysis. Proc. Natl. Acad. Sci. USA, 89:5847-5851.

Although the present invention and its advantages have been described in detail, it should be understood that various changes, substitutions and alterations can be made herein without departing from the spirit and scope of the invention as defined by the description provided herein. Moreover, the scope of the present application is not intended to be limited to the particular embodiments of the process, manufacture, and composition of matter, means, methods and steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the disclosure provided herein is intended to include within its scope such processes, machines, manufacture, compositions of matter, means, methods, or steps.

1. A method of preparing a DNA molecule, comprising:
obtaining at least one DNA molecule;
randomly fragmenting the DNA molecule to produce DNA fragments;
modifying the ends of the DNA fragments to provide attachable ends;
attaching an adaptor having at least one known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor;
extending the 3, end of the modified DNA from the nick site; and
amplifying a plurality of the adaptor-linked fragments.

2. The method of claim 1, wherein said at least one DNA molecule is further defined as genomic DNA.

3. The method of claim 1, wherein said modifying step is further defined as modifying the ends of the DNA fragments to comprise blunt double stranded ends.

4. The method of claim 1, wherein said modifying step is further defined as modifying the ends of the DNA fragments to comprise an overhang of at least 1 nucleotide.

5. The method of claim 1, wherein said randomly fragmenting the DNA molecule comprises mechanical fragmentation.

6. The method of claim 5, wherein said mechanical fragmentation comprises hydrodynamic shearing, sonication, nebulization, or a combination thereof.

7. The method of claim 1, wherein said randomly fragmenting the DNA molecule comprises chemical fragmentation.

8. The method of claim 7, wherein said chemical fragmentation comprises acid catalytic hydrolysis, alkaline catalytic hydrolysis, hydrolysis by metal ions, hydroxyl radicals, irradiation, heating, or a combination thereof.

9. The method of claim 1, wherein said randomly fragmenting the DNA molecule comprises enzymatic fragmentation.

10. The method of claim 9, wherein said enzymatic fragmentation comprises DNAse I digestion.

11. The method of claim 9, wherein said enzymatic fragmentation comprises Cvi JI restriction enzyme digestion.

12. The method of claim 8, wherein said chemical fragmentation comprises heating.

13. The method of claim 1, wherein the modifying step comprises repair of at least one 3′ end of the DNA fragment.

14. The method of claim 13, wherein the modifying step comprises subjecting said DNA fragment to 3′ exonuclease activity, 5′-3′ polymerase activity, or both.

15. The method of claim 14, wherein both of said 3, exonuclease activity and said 5′-3′ polymerase activity are comprised in the same enzyme.

16. The method of claim 15, wherein the enzyme comprises Klenow, T4 DNA polymerase, or a mixture thereof.

17. The method of claim 14, wherein the 3′ exonuclease activity comprises Exonuclease III activity and the 3′ polymerase activity comprises T4 DNA polymerase activity.

18. The method of claim 17, wherein following said subjecting step, said DNA fragments are subjected to Klenow, T4 DNA polymerase, or both.

19. The method of claim 7, wherein said DNA fragments comprise a plurality of ssDNA molecules and said modifying step is further defined as subjecting said ssDNA molecules to a plurality of random primers and DNA polymerase activity, under conditions wherein said blunt double stranded fragments are thereby generated.

20. The method of claim 19, wherein the random primers further comprise a known sequence at their 5′ end.

21. The method of claim 19, wherein at least one ssDNA molecule comprises a blocked 3′ end and wherein said modifying step is further defined as subjecting said ssDNA to 3′-5′ exonuclease activity.

22. The method of claim 19, wherein the random primers are pentamers.

23. The method of claim 19, wherein the random primers are hexamers.

24. The method of claim 19, wherein the random primers are septamers.

25. The method of claim 19, wherein the random primers are octamers.

26. The method of claim 19, wherein the random primers are nonamers.

27. The method of claim 19, wherein the random primers are phosporylated at the 5′ end.

28. The method of claim 19, wherein the random primers are comprised of at least one base analog, at least one backbone analog, or both.

29. The method of claim 19, wherein said DNA polymerase activity and said 3′-5′ exonuclease activity are comprised in the same enzyme.

30. The method of claim 19, wherein said polymerase is a non strand-displacing polymerase.

31. The method of claim 19, wherein said polymerase is a strand-displacing polymerase.

32. The method of claim 30, wherein said non strand-displacing polymerase is T4 DNA polymerase.

33. The method of claim 31, wherein said strand-displacing enzyme is Klenow or DNA polymerase I.

34. The method of claim 19, wherein said polymerase comprises nick translation activity.

35. The method of claim 19, wherein said enzyme is Klenow, T4 DNA polymerase, or DNA polymerase I, or a mixture thereof.

36. The method of claim 19, wherein said modifying step occurs in the presence of additives known to facilitate polymerization through GC-rich DNA.

37. The method of claim 36, wherein said additives comprise dimethyl sulfoxide (DMSO), 7-Deaza-dGTP, or a mixture thereof.

38. The method of claim 1, wherein said modifying step and said attaching step occurs concomitantly.

39. The method of claim 9, wherein said enzymatic fragmentation occurs in the presence of Mn²⁺ and said modifying step is further defined as subjecting said DNA fragments to 3′ exonuclease activity, 5′-3′ polymerase activity, or both.

40. The method of claim 39, wherein both of said 3′ exonuclease activity and said 5′-3′ polymerase activity are comprised in the same enzyme.

41. The method of claim 40, wherein said enzyme is Klenow, T4 DNA polymerase, or a mixture thereof.

42. The method of claim 40, wherein said 3′ exonuclease activity is by exonuclease III and said 5′-3′ polymerase activity is by T4 DNA polymerase.

43. The method of claim 42, wherein following said subjecting step, said DNA fragments are subjected to Klenow, T4 DNA polymerase, or both.

44. The method of claim 9, wherein said enzymatic fragmentation occurs in the presence of Mg²⁺ and said modifying step is further defined as subjecting said DNA fragments to random primers, 5′-3′ polymerase activity and 3′-5′ exonuclease activity.

45. The method of claim 44, wherein said 5′-3′ polymerase activity and said 3′-5′ exonuclease activity are comprised in the same enzyme.

46. The method of claim 45, wherein said enzyme is Klenow, T4 DNA polymerase, DNA polymerase I, or a mixture thereof.

47. The method of claim 1, wherein said attaching step is further defined as subjecting said DNA fragments to a blunt end adaptor, a 5′ overhang adaptor, a 3′ overhang adaptor, or a mixture thereof.

48. The method of claim 1, wherein said adaptor comprises at least one of the following features:
absence of a 5′ phosphate group;
a 5′ overhang; or
a blocked 3′ base.

49. The method of claim 48, wherein said 5′ overhang comprises about 5 to about 100 bases.

50. The method of claim 1, wherein said attaching is by ligating the adaptor to the DNA fragment.

51. The method of claim 50, wherein said ligation is by chemical ligation.

52. The method of claim 50, wherein said ligation is by enzymatic ligation.

53. The method of claim 52, wherein said enzymatic ligation is by T4 DNA ligase.

54. The method of claim 52, wherein said enzymatic ligation is by topoisomerase I.

55. The method of claim 54, wherein said adaptor is covalently attached to topoisomerase I at a 3′ thymidine overhang or a blunt end.

56. The method of claim 55, wherein said adaptor comprises a sequence of 5′-CCCTT-3′.

57. The method of claim 54, wherein the DNA fragments are blunt ended and a 3′ adenine is added to the blunt ended DNA fragments by polymerase.

58. The method of claim 1, wherein the adaptor comprises a first primer and a second primer, said first primer greater in length than said second primer.

59. The method of claim 58, wherein the second primer comprises a blocked 3′ end.

60. The method of claim 1, wherein the adaptor comprises at least one blunt end.

61. The method of claim 60, wherein the 3′ end of at least one primer is blocked.

62. The method of claim 50, wherein the adaptor comprises one oligonucleotide having two regions complementary to each other, said regions separated by a linker region.

63. The method of claim 62, wherein when the two complementary regions are hybridized to each other to form a double-stranded region of said adaptor, the end of said double stranded region is a blunt end.

64. The method of claim 62, wherein said linker region comprises a non-replicable organic chain of about 1 to about 50 atoms in length.

65. The method of claim 64, wherein said non-replicable organic chain is hexa ethylene glycole (HEG).

66. The method of claim 1, wherein said extending step comprises subjecting the adaptor-linked fragments comprising the nick to a mixture comprising:
DNA polymerase;
deoxynucleotide triphosphates; and
suitable buffer, under conditions wherein polymerization occurs from the 3′ hydroxyl of the nick.

67. The method of claim 66, wherein the method further comprises heating the mixture.

68. The method of claim 67, wherein said heating is to a temperature of about 75° C.

69. The method of claim 66, wherein the polymerase is a strand-displacing polymerase.

70. The method of claim 66, wherein the DNA polymerase is a thermophilic DNA polymerase.

71. The method of claim 70, wherein the thermophilic DNA polymerase is Taq polymerase.

72. The method of claim 66, wherein at least one deoxynucleotide triphosphate is labeled.

73. The method of claim 1, wherein said amplifying step comprises polymerase chain reaction, said reaction utilizing a primer complementary to a sequence of the adaptor.

74. The method of claim 73, wherein said primer is labeled.

75. The method of claim 1, wherein said amplifying step occurs in the presence of additives known to facilitate polymerization through GC-rich DNA.

76. The method of claim 75, wherein said additives comprise DMSO, 7-Deaza-dGTP, or a mixture thereof.

77. The method of claim 1, wherein said at least one DNA molecule is comprised in a cell.

78. The method of claim 1, wherein said at least one DNA molecule is not comprised in a cell.

79. The method of claim 77, wherein the at least one DNA molecule is cell-free fetal DNA in maternal blood or is cell-free cancer DNA in blood.

80. The method of claim 1, wherein said obtaining method is further defined as obtaining the at least one DNA molecule from blood, urine, sputum, feces, sweat, nipple aspirate, a fixed tissue sample, immuno-precipitated chromatin, physically isolated chromatin, or a combination thereof.

81. The method of claim 80, wherein said physically isolated chromatin is isolated by centrifugation, electrophoresis, micro-filtration, affinity capture, or a combination thereof.

82. The method of claim 2, wherein said genomic DNA comprises bacterial genomic DNA, viral genomic DNA, fungal genomic DNA, plant genomic DNA, or mammalian genomic DNA.

83. The method of claim 2, wherein said genomic DNA is from an extant species or an extinct species.

84. The method of claim 1, wherein said at least one DNA molecule comprises a portion of a genome.

85. The method of claim 1, wherein said adaptor is further defined as a first adaptor having a first known sequence and further comprises a homopolymeric sequence, the method further comprising the following steps:
digesting the amplified adaptor-linked fragments to produce fragmented adaptor-linked fragments;
attaching a second adaptor having a second known sequence to the ends of the fragmented adaptor-linked fragments to produce second adaptor-linked fragments; and
amplifying the second adaptor-linked fragments with a primer complementary to the homopolymeric sequence and a primer complementary to the second known sequence.

86. The method of claim 85, wherein said homopolymeric sequence is comprised of cytosines.

87. The method of claim 1, wherein said adaptor is further defined as a first adaptor having a first known sequence, the method further comprising the following steps:
subjecting the amplified adaptor-linked fragments to terminal deoxynucleotidyl transferase to generate a homopolymeric single-stranded tail on said amplified adaptor-linked fragments;
digesting the homopolymeric tailed amplified adaptor-linked fragments;
attaching a second adaptor having a second known sequence to the ends of the digested homopolymeric tailed amplified adaptor-linked fragments that do not comprise the homopolymeric tail, to produce second adaptor-linked fragments; and
amplifying the second adaptor-linked fragments with a primer complementary to the homopolymeric sequence and a primer complementary to the second known sequence.

88. A method of preparing a DNA molecule, comprising:
obtaining at least one DNA molecule;
attaching a first adaptor having a first known sequence, a homopolymeric sequence and a nonblocked 3′ end to the ends of the DNA molecule to produce first adaptor-linked molecules, wherein the 5′ end of the DNA molecule is attached to the nonblocked 3′ end of the adaptor, leaving a nick site between the juxtaposed 3′ end of the DNA molecule and a 5′ end of the adaptor;
digesting the adaptor-linked DNA molecules to produce DNA fragments;
attaching a second adaptor having a second known sequence to the ends of the DNA fragments to produce second adaptor-linked fragments; and
amplifying a plurality of the second adaptor-linked fragments.

89. A method of preparing a DNA molecule, comprising:
obtaining a plurality of DNA molecules, said DNA molecules defined as fragments from at least one larger DNA molecule;
modifying the ends of the DNA fragments to provide attachable ends;
attaching an adaptor having a known sequence and a nonblocked 3′ end to both ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor;
extending the 3′ end of the modified DNA from the nick site; and
amplifying a plurality of the adaptor-linked fragments.

90. The method of claim 89, wherein said at least one larger DNA molecule comprises genomic DNA.

91. A method of amplifying a genome, comprising the steps of:
obtaining at least one DNA molecule;
randomly fragmenting the DNA molecule to produce DNA fragments;
modifying the ends of the DNA fragments to provide attachable ends;
attaching an adaptor having a known sequence and a nonblocked 3, end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor;
extending the 3′ end of the modified DNA from the nick site; and
amplifying a plurality of the adaptor-linked fragments.

92. A method of generating a library, comprising the steps of:
obtaining at least one DNA molecule;
randomly fragmenting the DNA molecule to produce DNA fragments;
modifying the ends of the DNA fragments to provide attachable ends;
attaching an adaptor having a known sequence and a nonblocked 3′ end to both ends of a plurality of the modified DNA fragments to produce adaptor-linked fragments, wherein the 5′ end of the modified DNA is attached to the nonblocked 3′ end of the adaptor, leaving a nick site between the juxtaposed 3′ end of the DNA and 5′ end of the adaptor;
extending the 3′ end of the modified DNA from the nick site.

93. The method of claim 92, wherein said method further comprises amplifying a plurality of the adaptor-linked fragments.

94. A method of preparing at least one DNA molecule, comprising:
admixing together:
an endonuclease;
a ligase;
an adaptor; and
a buffer, under conditions wherein said DNA molecule is cleaved by said endonuclease to generate a plurality of DNA fragments, a plurality of the ends of which are ligated to said adaptor.

95. The method of claim 94, wherein the method consists essentially of one step.

96. The method of claim 94, wherein the cleavage and ligation occur substantially concomitantly.

97. The method of claim 94, further defined as the ligation occurring under the same reaction conditions as the cleavage.

98. The method of claim 94, wherein the ligation step occurs without changing the buffer following the cleavage step.

99. The method of claim 94, wherein the method lacks DNA precipitation.

100. The method of claim 94, wherein said DNA molecule is further defined as a genome.

101. The method of claim 94, wherein said endonuclease is deoxyribonuclease I or a Cvi restriction endonuclease.

102. The method of claim 94, wherein said ligase is T4 DNA ligase.

103. The method of claim 94, wherein said adaptor is a blunt end adaptor, a 5, overhang adaptor, a 3, overhang adaptor, or a mixture thereof.

104. The method of claim 94, wherein the adaptor comprises a first primer and a second primer, said first primer greater in length than said second primer.

105. The method of claim 104, wherein said first primer lacks a 5′ phosphate, said second primer lacks a 5′ phosphate group, or both first and second primers lack 5′ phosphate groups.

106. The method of claim 94, wherein the buffer comprises a divalent cation, a salt, adenosine triphosphate, dithiothreitol, or a mixture thereof.

107. The method of claim 94, wherein the conditions comprise a large molar excess of linkers to DNA fragment ends.

108. The method of claim 107, wherein the large molar excess is at least about 10-fold to about 100-fold.

109. The method of claim 94, wherein said method further comprises amplifying the DNA fragments using a primer complementary to the adaptor.

110. A method of generating a library of DNA molecules comprising:
admixing together:
at least one DNA molecule;
an endonuclease;
a ligase;
an adaptor; and
a buffer, under conditions wherein said DNA molecule is cleaved by said endonuclease to generate a plurality of DNA fragments, a plurality of the ends of which are ligated to said adaptor.

111. The method of claim 110, wherein said method consists essentially of one step.

112. A kit for performing a concomitant endonuclease/ligase reaction, comprising:
an endonuclease;
a ligase;
an adaptor; and
a buffer.

113. The kit of claim 112, wherein the adaptor is a blunt end adaptor, a 5′ overhang adaptor, a 3′ overhang adaptor, or a mixture thereof.

114. The kit of claim 112, wherein the adaptor comprises a first primer and a second primer, said first primer greater in length than said second primer.

115. The kit of claim 114, wherein said first primer lacks a 5′ phosphate, said second primer lacks a 5′ phosphate group, or both first and second primers lack 5′ phosphate groups.

116. A method of diagnosing a condition in an individual, comprising the step of:
obtaining at least one DNA molecule from said individual;
randomly fragmenting the DNA molecule to produce DNA fragments;
modifying the ends of the DNA fragments to provide attachable ends;
attaching an adaptor having a known sequence and a nonblocked 3′ end to the ends of the modified DNA fragments to produce adaptor-linked fragments, wherein the 5′ end of the DNA is attached to the nonblocked 3′ end of the adaptor, leaving a nick site between the juxtaposed 3′ end of the DNA and a 5′ end of the adaptor;
extending the 3′ end of the modified DNA from the nick site;
amplifying at least one adaptor-linked fragment; and
identifying a DNA sequence in said fragment that is representative of said condition.

117. The method of claim 116, wherein said DNA sequence in said fragment comprises at least a portion of an X chromosome or a Y chromosome.

118. The method of claim 116, wherein said DNA sequence is a point mutation, a deletion, an inversion, a repeat, or a combination thereof.

119. A method of amplifying at least one RNA molecule, comprising the steps of:
obtaining at least one RNA molecule;
reverse transcribing said RNA molecule to produce a cDNA molecule;
randomly fragmenting the cDNA molecule to produce DNA fragments;
modifying the ends of the DNA fragments to provide attachable ends;
attaching an adaptor having a known sequence and a nonblocked 3′ end to the ends of the modified DNA fragments to produce adaptor-linked fragments, wherein the 5′ end of the DNA is attached to the nonblocked 3′ end of the adaptor, leaving a nick site at the juxtaposed 3′ end of the DNA and a 5′ end of the adaptor;
extending the 3′ end of the modified DNA from the nick site; and
amplifying a plurality of the adaptor-linked fragments.

120. A method of amplifying a population of DNA molecules comprised in a plurality of populations of DNA molecules, said method comprising the steps of:
obtaining a plurality of populations of DNA molecules, wherein at least one population in said plurality comprises DNA molecules having in a 5′ to 3′ orientation the following:
a known identification sequence specific for said population; and
a known primer amplification sequence; and
amplifying said population of DNA molecules by polymerase chain reaction, said reaction utilizing a primer for said identification sequence.

121. The method of claim 120, wherein said obtaining step is further defined as:
obtaining a population of DNA molecules, said molecules comprising a known primer amplification sequence;
amplifying said DNA molecules with a primer having in a 5′ to 3′ orientation the following:
the known identification sequence; and
the known primer amplification sequence; and
mixing said population with at least one other population of DNA molecules.

122. The method of claim 120, wherein said population of DNA molecules is a genome.

123. A method of amplifying a population of DNA molecules comprised in a plurality of populations of DNA molecules, said method comprising the steps of:
obtaining a plurality of populations of DNA molecules, wherein at least one population in said plurality comprises DNA molecules, wherein the 5′ ends of said DNA molecules comprise in a 5′ to 3′ orientation the following:
a single-stranded region comprising a known identification sequence specific for said population; and
a known primer amplification sequence; and
isolating said population through binding of at least part of the single stranded known identification sequence of a plurality of said DNA molecules to a surface; and
amplifying the isolated DNA molecules by polymerase chain reaction, said reaction utilizing a primer for said primer amplification sequence.

124. The method of claim 123, wherein said obtaining step is further defined as:
obtaining a population of DNA molecules, said molecules comprising a known primer amplification sequence;
amplifying said DNA molecules with a primer comprising in a 5′ to 3′ orientation the following:
the known identification sequence;
a non-replicable linker; and
the known primer amplification sequence; and
mixing said population with at least one other population of DNA molecules.

125. The method of claim 123, wherein said isolating step is further defined as binding at least part of the single stranded known identification sequence to an immobilized oligonucleotide comprising a region complementary to the known identification sequence.

126. A method of immobilizing an amplified genome, comprising the steps of:
obtaining an amplified genome, wherein a plurality of DNA molecules from the genome comprise a known primer amplification sequence at both the 5′ and 3′ ends of the molecules; and
attaching a plurality of the DNA molecules to a support.

127. The method of claim 126, wherein said attaching step is further defined as comprising covalently attaching the plurality of DNA molecules to the support through said known primer amplification sequence.

128. The method of claim 126, wherein said covalently attaching step is further defined as:
hybridizing a region of at least one single stranded DNA molecules to a complementary region in the 3′ end of a oligonucleotide immobilized to said support; and
extending the 3′ end of the oligonucleotide to produce a single stranded DNA/extended polynucleotide hybrid.

129. The method of claim 128, wherein said method further comprises the step of removing the single stranded DNA molecule from the single stranded DNA/extended polynucleotide hybrid to produce an extended polynucleotide.

130. The method of claim 128, wherein said method further comprises the step of replicating the extended polynucleotide.

131. The method of claim 130, wherein said replicating step is further defined as:
providing to said extended polynucleotide a DNA polymerase and a primer complementary to the known primer amplification sequence;
extending the 3′ end of said primer to form an extended primer molecule; and
releasing said extended primer molecule.

132. A method of immobilizing an amplified genome, comprising the steps of:
obtaining an amplified genome, wherein a plurality of DNA molecules from the genome comprise:
a tag; and
a known primer amplification sequence at both the 5, and 3′ ends of the molecules; and
attaching a plurality of the DNA molecules to a support.

133. The method of claim 132, wherein said attaching step is further defined as comprising attaching the plurality of DNA molecules to the support through said tag.

134. The method of claim 132, wherein said tag is biotin said said support comprises streptavidin.

135. The method of claim 132, wherein said tag is an amino group or a carboxy group.

136. The method of claim 132, wherein said tag comprises a single stranded region and said support comprises an oligonucleotide comprising a sequence complementary to a region of said tag.

137. The method of claim 136, wherein said single stranded region is further defined as comprising an identification sequence.

138. The method of claim 137, wherein said DNA molecules are further defined as comprising a non-replicable linker that is 3′ to said identification sequence and that is 5′ to said known primer amplification sequence.

139. The method of claim 132, wherein said method further comprises the steps of removing contaminants from the immobilized genome.

140. A method of preparing a DNA molecule, comprising:
obtaining a population of DNA molecules having ligatable ends of unknown nature;
providing to said population one or more known forms of adaptors, wherein said adaptors each comprise at least one known sequence and at least one oligonucleotide having a 3′ extendable end;
determining ligatability of said one or more known forms of adaptors to said DNA molecules; and
ligating said known one or more forms of adaptors to said DNA molecule.

141. The method of claim 140, wherein said determining step is further defined as identifying a ratio of ligatable forms of adaptors corresponding to the nature of the ends of the DNA molecules in the population, and wherein said ligating step is further defined as introducing to said population a plurality of said adaptors in said ratio.

142. The method of claim 140, wherein said ligatability of said one or more forms of adaptors are determined separately.

143. The method of claim 140, wherein said method further comprises the step of extending the 3′ end of said oligonucleotide by polymerization to produce an extended product.

144. The method of claim 143, wherein said method further comprises the step of amplifying said extended product by polymerase chain reaction.

145. The method of claim 140, wherein said population of DNA molecules is obtained from serum.

146. The method of claim 140, wherein said population of DNA molecules is obtained from plasma.

147. A method of sequencing genomic DNA from a limited source of material, comprising the steps of:
obtaining at least one DNA molecule from a limited source of material;
randomly fragmenting the DNA molecule to produce DNA fragments;
modifying the ends of the DNA fragments to provide attachable ends;
attaching an adaptor having a known sequence and a nonblocked 3′ end to the ends of the modified DNA fragments to produce adaptor-linked fragments, wherein the 5′ end of the modified DNA is attached to the nonblocked 3′ end of the adaptor, leaving a nick site between the juxtaposed 3′ end of the DNA and a 5′ end of the adaptor;
extending the 3′ end of the modified DNA from the nick site;
amplifying a plurality of the adaptor-linked fragments;
providing from the plurality of the adaptor-linked fragments a first sample of adaptor-linked fragments and a second sample of adaptor-linked fragments;
sequencing at least some of the adaptor-linked fragments from the first sample;
incorporating homopolymeric sequence to the ends of the adaptor-linked fragments from the second sample;
amplifying at least some of the adaptor-linked fragments from the second sample utilizing a first primer complementary to the homopolymeric sequence and a second primer complementary to a specific sequence in the adaptor-linked fragments from the second sample; and
analyzing at least some of the amplified sequence.

148. The method of claim 147, wherein said incorporating of the homopolymeric sequence comprises one of the following steps:
extending the 3′ end of the adaptor-linked fragments by terminal deoxynucleotidyl transferase;
ligating an adaptor comprising the homopolymeric sequence to the ends of the adaptor-linked fragments; or
replicating the adaptor-linked fragments with a primer comprising the homopolymeric sequence at its 5′ end.

149. The method of claim 147, wherein said sequencing step is further defined as:
cloning the adaptor-linked fragments from the first sample into a vector; and
sequencing at least some of the cloned adaptor-linked fragments from the first sample.

150. The method of claim 147, wherein the specific sequence of the DNA molecule is provided by the sequencing step of the adaptor-linked fragments from the first sample.

151. The method of claim 147, wherein said limited source of material is a microorganism substantially resistant to culturing.

152. The method of claim 147, wherein said limited source of material is an extinct species.
